(19) United States
(12) Patent Application Publication
(10) Pub. No.: US 2020/0246365 A1
(43) Pub. Date: Aug. 6, 2020

(54) COMPOUNDS, PHARMACEUTICAL COMPOSITIONS AND USE THEREOF AS INHIBITORS OF RAN GTPASE

(71) Applicants: THE ROYAL INSTITUTION FOR THE ADVANCEMENT OF LEARNING / MCGILL UNIVERSITY, Montreal (CA); VAL-CHUM, LIMITED PARTNERSHIP, Montreal (CA)

(72) Inventors: JIAN HUI WU, MONTREAL (CA); GERALD BATIST, MONTREAL (CA); XIAOCHONG TIAN, URUMQI (CN); XIAOLONG LI, JIANYANG (CN); ANNE-MARIE MES-MASSON, DOLLARD-DES-ORMEAUX (CA); DIANE PROVENCHER, ST-BASILE-LE-GRAND (CA); EURIDICE CARMONA, POINTE-CLAIRE (CA)

(21) Appl. No.: 16/643,345

(22) PCT Filed: Aug. 30, 2018
(86) PCT No.: PCT/CA2018/051045

§ 371 Date: Feb. 28, 2020

Related U.S. Application Data
(60) Provisional application No. 62/554,150, filed on Sep. 5, 2017.

(51) Int. Cl. A61K 31/7034; A61K 33/243; A61K 31/501; A61K 31/166; A61K 31/135; A61K 31/14; A61K 31/444; A61K 31/09; A61K 31/075; A61K 31/341; A61K 31/381; A61K 31/277; A61K 31/17; A61K 31/18; A61K 31/41; A61K 31/426; A61K 31/495; A61K 31/496; A61P 35/00; C07H 15/207; C07C 233/66; C07C 211/27; C07C 43/205; C07D 401/14; C07D 407/14; C07D 409/14; C07C 255/58; C07C 275/40; C07D 257/04; C07D 277/56; C07D 241/04; C07D 401/06

(52) U.S. Cl. CPC A61K 31/7034; C07D 401/06; A61K 31/501; A61K 31/166; A61K 31/135; A61K 31/14; A61K 31/444; A61K 31/09; A61K 31/075; A61K 31/341; A61K 31/381; A61K 31/277; A61K 31/17; A61K 31/18; A61K 31/41; A61K 31/426; A61K 31/495; A61K 31/496; A61P 35/00; C07H 15/207; C07C 233/66; C07C 211/27; C07C 43/2055; C07D 401/14; C07D 407/14; C07D 409/14; C07C 255/58; C07C 275/40; C07D 257/04; C07D 277/56; C07D 241/04; A61K 33/243

(57) ABSTRACT

Compounds of general formula IA, IB and IC outlined below, including pharmaceutically acceptable salts, solvates and hydrates thereof. Such compounds and pharmaceutical compositions comprising them may be used in medical conditions involving Ran GTPase.

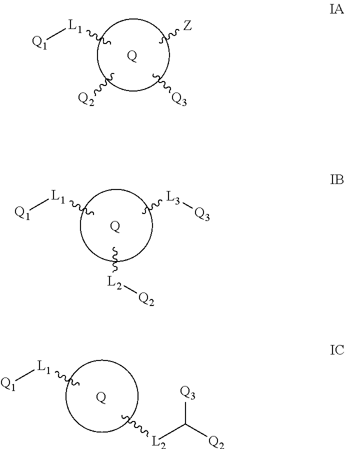

### CROSS REFERENCE TO RELATED APPLICATIONS

This application claims benefit of U.S. Provisional Application No. 62/554,150 filed Sep. 5, 2017, which is incorporated herein in its entirety by reference.

### FIELD OF THE INVENTION

The present invention relates generally to medical conditions involving Ran GTPase. More specifically, the invention relates to compounds and pharmaceutical compositions comprising such compounds for use in the inhibition of Ran GTPase.

### BACKGROUND OF THE INVENTION

Ovarian cancer is the most lethal gynecologic malignancies in North America, with a five-year survival rate of 45% [1]. The most common form is epithelial ovarian cancer (EOC), where ˜70% of EOC patients present with a high-grade serous (HGS) histotype [2]. Standard first line therapy of EOC consists of tumor cytoreductive surgery and treatment with platinum DNA alkylating agents such as carboplatin or cisplatin combined with the microtubule poison paclitaxel [3]. Although initial response rates are high (>70%), the disease eventually recurs in most patients, who will develop chemoresistance [3,4]. Over the past 45 years, advances in surgery and chemotherapy have had little impact on overall patient survival [3,4] underscoring the need for the development of new clinical tools for the management of EOC patients.

The extensive genome sequencing studies have revealed that HGS EOC presents extremely high intra-tumoral heterogeneity (ITH) [5], which poses specific challenges for therapeutic strategies. In particular, within the same tumor some specific cell populations may be drug-resistant (or become drug-resistant) leading to patient relapse. Furthermore, it has been shown that the diversity and the heterogeneity of HGS EOC arises at early stages of the tumorigenic process, and that metastatic sites in the same individual have different cell populations with distinct molecular features [5]. This ITH is now recognized as a hallmark of HGS EOC and presents specific challenges for therapeutic strategies. However, independently of their heterogeneity, these EOC cancer cell populations have complex karyotypes and aneuploidy [5-9]. Therefore, a strategy that specifically targets aneuploidy would be successful in treating this cancer, including carboplatin resistant cells.

Our group has a longstanding interest in fundamental, translational and clinical research in EOC, and we have established an ovarian tissue repository (including fresh frozen specimens from normal ovaries, benign and tumor tissues, primary cultures from clinical material and paraffin embedded samples). We have also invested efforts into the establishment of spontaneously immortalized long-term cell lines derived from tumor or ascites from chemotherapy naive patients or from disease recurrence after treatment [7,10-12], including a number of HGS EOC cell lines. Using these resources our laboratory has generated high-throughput datasets such as gene and tissue microarrays, as well as next generation sequencing and copy number variation analysis. Using these datasets we have identified genes modulated during the course of EOC initiation and progression [13-24] and have characterized biological parameters that are affected by the modulation of candidates using both in vitro and in vivo assays [16,18,23,25,26]. We have also shown that our HGS cell lines have several mutations and gene expression de-regulation in G2/M cell cycle genes and in genes associated with DNA repair [10], which might be involved in the HGS genomic instability.

The small GTPase Ran (Ras-related nuclear protein) is a promising candidate biomarker of therapeutic value identified by our transcriptome, tissue array and molecular analyses [20,25,27,28]. Its importance in cancer progression of other tissue types has also been described [29-33]. These studies, including our own (FIG. 1), have all reported that Ran overexpression is associated with malignancy and with poor patient prognosis. Ran performs two major and distinct cellular functions. During interphase, Ran regulates nucleo-cytoplasmic transport of molecules through the nuclear pore complex [34,35]. At mitosis, Ran controls cell cycle progression through the regulation of mitotic spindle formation [36]. The Ran-GTP/GDP cycle is regulated by several proteins [37-40], which are involved in both physiological functions of Ran through different gradients [41]. We have shown that down-regulation of Ran induced cell death by apoptosis and decreased cell proliferation and in vivo tumor growth of EOC cells [25] (FIG. 2). Similar findings have been reported in other cancer types [31,42-44].

In contrast, Ran siRNA knockdown does not induce apoptosis in a range of normal cell types [31,44]. Interestingly, in support of our findings it has been described that Ran is an essential survival gene as revealed by shRNA functional screening assays of ovarian, breast and pancreatic cancer cell lines [45,46], demonstrating its potential as a therapeutic target for multiple tumor types including EOC.

A recent study has shown that tumor cells have a steeper mitotic Ran-GTP gradient than normal cells resulting in altered prometaphase/metaphase timing [47], that in turn can influence cell proliferation [48]. This report also showed that a steep Ran-GTP gradient could be induced by chromosomal gain [47]. Therefore, aneuploidy in tumor cells is associated with a Ran-GTP gradient at mitosis, and these results may explain the findings that Ran knockdown induced cell death in cancer but not in normal cells [25,31,42,43].

Hence, we postulated that HGS cells are more sensitive to Ran down-regulation due to their extensive chromosomal anomalies and aneuploidy, and that a therapeutic index between normal and cancer cells to Ran loss can be defined. Targeting GTPase Ran with small molecules inhibitors would be a new strategy to treat EOC and other cancer types with aberrant chromosome number. To date, no chemical inhibitors of Ran have been reported, despite the availability of Ran's crystal structure.

There is a need to develop compounds that are inhibitors of Ran GTPase. Also, there is a need to investigate the use of such compounds in the treatment of medical conditions involving Ran GTPase.

### SUMMARY OF THE INVENTION

The inventors have designed and prepared novel chemical compounds that are small molecules. The compounds according to the invention inhibit Ran GTPase and may be used in the treatment of medical conditions involving Ran GTPase. Such medical conditions may be for example cancers including ovarian cancer, breast cancer, pancreatic cancer, colorectal cancer and cancers embodying aneuploidy.

More specifically, the inventors have investigated the therapeutic value of the compounds according to the invention using in vitro and in vivo epithelial ovarian cancer (EOC) models that they have designed.

Also, the compounds according to the invention may be used in association with other therapeutic agents, which may be for example, DNA damaging agents such as carboplatin, inhibitors of poly ADP ribase polymerase (PARP) such as olaparib.

The invention thus provides the following in accordance with aspects thereof:

(1) A compound of general formula IA below, or a pharmaceutically acceptable salt thereof, or a solvate or hydrate thereof

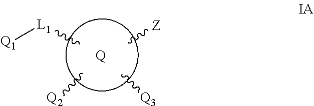

wherein:
Q is a 5 to 20-member single or multicyclo ring comprising at least one of O and S atoms;
L is a group comprising one or more of (CH₂), (CH), O, S, and C═X wherein X is O or S;
Z is CN; or (C═X)NR₁R₂ wherein X is O or S and R₁ and R₂ are each independently selected from H, alkyl, cycloalkyl, alkene, alkyne, aryl, alkylaryl, or together R₁ and R₂ form a 3 to 6-member ring which is optionally substituted with a substituent selected from alkyl, OH, SH, NH₂, a halogen atom, CN, NO₂ and SO₂; or a 3 to 6-member ring comprising one or more heteroatoms which are the same or different, optionally the ring is substituted with a substituent selected from COOR wherein R is a C₁-C₆-alkyl or cycloalkyl, alkoxy, alkyl, OH, SH, NH₂, a halogen atom, CN, NO₂ and SO₂; and
Q₁, Q₂ and Q₃ are each independently selected from alkyl, cycloalkyl, alkene, alkyne, aryl and alkylaryl, a 5 to 12-member single or bicyclo ring; optionally, the ring is substituted with a substituent selected from alkyl, cycloalkyl alkoxy, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂ and Se(═O)₂; also optionally, the ring comprises one or more heteroatoms which are the same or different; and
the heteroatom is selected from O, N, S and Se.
(2) A compound according to (1) having the general formula IIA below

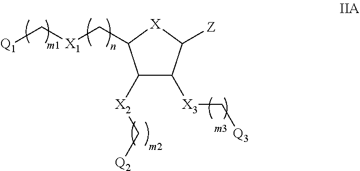

wherein:
Z is (C═X)NR₁R₂ or a 5-member ring comprising two heteroatoms which are different and the ring is substituted with COOR;

### X is O, N or S;

n, m1, m2, and m3 are each independently an integer from 1 to 6; and
X₁, X₂ and X₃ are each independently O, N or S.
(3) A compound according to (2) having the general formula IIIA below

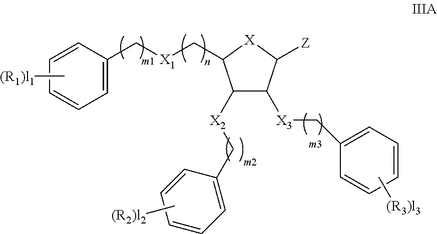

wherein:
Z is (C═X)NR₁R₂ or a 5-member ring comprising two heteroatoms which are different and the ring is substituted with COOR;
R₁, R₂ and R₃ are each independently H, alkyl, cycloalkyl, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂ and Se(═O)₂; and
I1, I2 and I3 are each independently an integer from 0 to 5.
(4) A compound according to (2) or (3), wherein X is O.
(5) A compound according to any one of (2) to (4), wherein Z is (C═S)NH₂ or

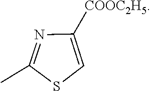

(6) A compound according to any one of (2) to (5), wherein X₁, X₂ and X₃ are each O.
(7) A compound according to any one of (2) to (6), wherein n, m1, m2 and m3 are each 1.
(8) A compound according to any one of (3) to (7), wherein R₁, R₂ and R₃ are each independently a halogen atom; and I1, I2 and I3 are each 1.
(9) A compound according to (1), wherein Q is the tetrahydrofuran ring.
(10) A compound according to (1), which is selected from the group of compounds depicted in the Table 1 herein below.
(11) A compound according to (1), which is compound M36 depicted below

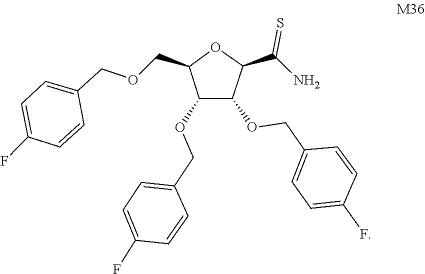 [SMILES: NC(=S)[C@@H]1O[C@H](COCc2ccc(F)cc2)[C@@H](OCc2ccc(F)cc2)[C@H]1OCc1ccc(F)cc1]

(12) A compound according to (1), which is compound M88 depicted below

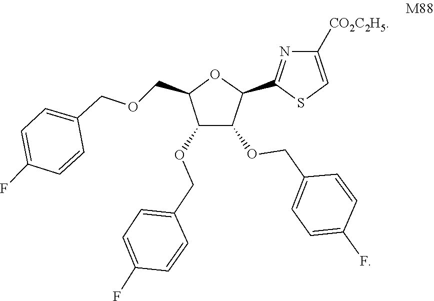 [SMILES: CCOC(=O)c1csc([C@@H]2O[C@H](COCc3ccc(F)cc3)[C@@H](OCc3ccc(F)cc3)[C@H]2OCc2ccc(F)cc2)n1]

(13) A compound of general formula IB below, or a pharmaceutically acceptable salt thereof, or a solvate or hydrate thereof

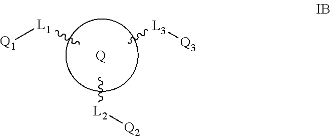

wherein:
Q is a 6 to 20-member single or multicyclo ring;
L₁, L₂ and L₃ are each independently a group comprising one or more of (CH₂), (CH), O, N, S and C═X wherein X is O or S, and NR₁R₂ wherein R₁ and R₂ are each independently selected from H, alkyl, cycloalkyl, alkene, alkyne, aryl, alkylaryl, or together R₁ and R₂ form a 3 to 6-member ring; and
Q₁, Q₂ and Q₃ are each independently selected from alkyl, cycloalkyl, alkene, alkyne, aryl and alkylaryl, a 5 to 12-member single or bicyclo ring; optionally, the ring is substituted with a substituent selected from alkyl, cycloalkyl alkoxy, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂ and Se(═O)₂; also optionally, the ring comprises one or more heteroatoms which are the same or different and selected from O, N, S and Se.
(14) A compound according to (13) having the general formula IIB or IIB′ below

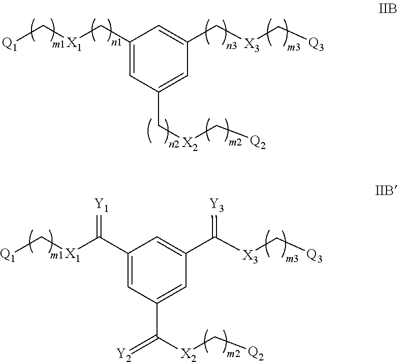

wherein:
n1, n2, n3, m1, m2, and m3 are each independently an integer from 0 to 6;
X₁, X₂ and X₃ are each independently selected from O; N; S; C═X wherein X is O or S; NR₁R₂ wherein R₁ and R₂ are each independently selected from selected from H, alkyl, cycloalkyl, alkene, alkyne, aryl, alkylaryl, or together R₁ and R₂ form a 3 to 6-member ring; (C═X)NR₁R₂ wherein X is O or S and R₁ and R₂ are each independently selected from selected from H, alkyl, cycloalkyl, alkene, alkyne, aryl, alkylaryl, or together R₁ and R₂ form a 3 to 6-member ring; and
Y₁, Y₂ and Y₃ are each independently selected from O, N and S.
(15) A compound according to (13) or (14) having the general formula IIIB or IIIB′ below

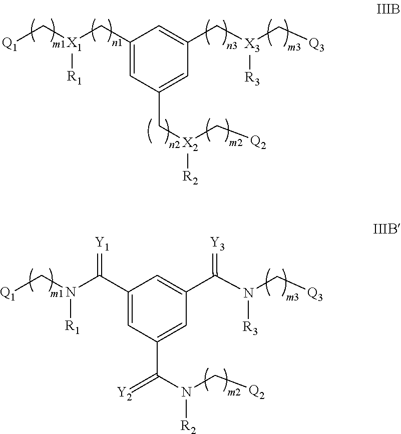

wherein:
X₁, X₂ and X₃ are each independently selected from O and N;
Y₁, Y₂ and Y₃ are each independently selected from O and S; and
R₁, R₂ and R₃ are each independently selected from H, alkyl, cycloalkyl, alkene, alkyne, aryl and alkylaryl.
(16) A compound according to (14) or (15), wherein n1, n2, n3, m1, m2, and m3 are each 1.
(17) A compound according to any one of (14) to (16), wherein X₁, X₂ and X₃ in IIB or IIIB are each N, and Y₁, Y₂ and Y₃ in IIB′ or IIIB′ are each O.
(18) A compound according to any one of (14) to (17), wherein Q₁, Q₂ and Q₃ are each independently is a 5 or 6-member ring, optionally the ring comprises one or more heteroatoms selected from O, N, S and Se, and/or optionally the ring is substituted with one or more groups selected from C₁ to C₆ alkoxy and halogens.
(19) A compound according to (13), wherein Q is the benzene ring.
(20) A compound according to (13), which is selected from the group of compounds depicted in the Table 2 herein below.
(21) A compound according to (13), which is compound M51 depicted below

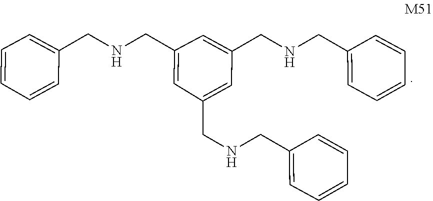 [SMILES: c1ccc(CNCc2cc(CNCc3ccccc3)cc(CNCc3ccccc3)c2)cc1]

(22) A compound according to (13), which is compound M55 depicted below

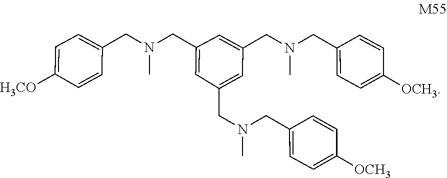 [SMILES: COc1ccc(CN(C)Cc2cc(CN(C)Cc3ccc(OC)cc3)cc(CN(C)Cc3ccc(OC)cc3)c2)cc1]

(23) A compound according to (13), which is compound M66 depicted below

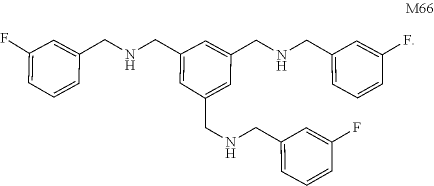 [SMILES: Fc1cccc(CNCc2cc(CNCc3cccc(F)c3)cc(CNCc3cccc(F)c3)c2)c1]

(24) A compound of general formula IC below, or a pharmaceutically acceptable salt thereof, or a solvate or hydrate thereof

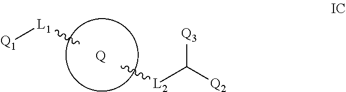

wherein:
Q is a 6 to 20-member single or multicyclo ring comprising at least two N atoms;
L₁ is a group comprising one or more of (CH₂), (CH), O, N, S and C═X wherein X is O or S, and L₁ is attached to one of the at least two N atoms;
L2 is present or absent and is a group comprising one or more of (CH₂), (CH), O, N, S and C═X wherein X is O or S, and L₂ is attached to another one of the at least two N atoms;
Q₁, Q₂ and Q₃ are each independently selected from H, alkyl, cycloalkyl, alkene, alkyne, aryl and alkylaryl, a 5 to 12-member single or bicyclo ring; optionally, the ring is substituted with a substituent selected from alkyl, cycloalkyl alkoxy, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂, Se(═O)₂ and N(HNC═X)₂(Ph-halogen(s))₂ wherein X is O or S; also optionally, the ring comprises one or more heteroatoms which are the same or different and selected from O, N, S and Se.
(25) A compound according to (24) having the general formula IIC below

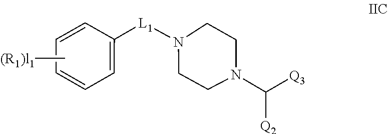

wherein:
Q₂ and Q₃ are each independently selected from alkyl, cycloalkyl, alkene, alkyne, aryl and alkylaryl, a 5 to 12-member single or bicyclo ring; optionally, the ring is substituted with a substituent selected from alkyl, cycloalkyl alkoxy, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂ and Se(═O)₂; also optionally, the ring comprises one or more heteroatoms which are the same or different and selected from O, N, S and Se;
R₁ is selected from H, alkyl, cycloalkyl, alkylaryl, alkoxy, thioalkoxy, aryl, aryloxy, thioaryloxy, alkylaryloxy, thioalkylaryloxy, OH, SH, NH₂, a halogen atom, a halogeno alkyl, a halogeno alkoxy, a halogeno thioalkoxy, CN, NO₂, S(═O)₂ and Se(═O)₂; and I1 is an integer from 0 to 5.
(26) A compound according to (25), having the general formula IIIC below

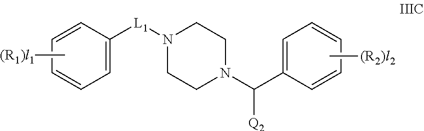

wherein: R₂ is as defined for R₁; and I2 is as defined for I1.
(27) A compound according to any one of (24) to (26), wherein L₁ is (CH₂)ₙ wherein n is an integer from 0 to 12.
(28) A compound according to (25) or (26), wherein Q₂ is a cycloalkyl or alkylaryl.
(29) A compound according to (24), wherein Q is the piperazine ring.
(30) A compound according to (24), which is selected from the group of compounds depicted in the Table 3 or Table 4 herein below.
(31) A compound according to (24), which is compound R20 depicted below

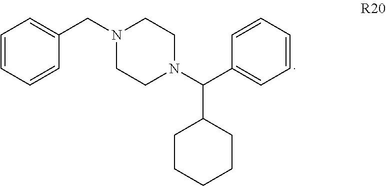 [SMILES: c1ccc(CN2CCN(C(c3ccccc3)C3CCCCC3)CC2)cc1]

(32) A compound according to (24), which is compound QR20 depicted below

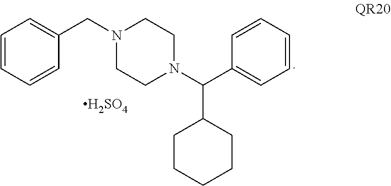

(33) A compound according to (24) having the general formula IIC′ below

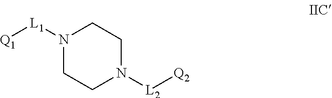

wherein: Q₁ and Q₂ are each independently selected from alkyl, cycloalkyl, alkene, alkyne, aryl and alkylaryl, a 5 to 12-member single or bicyclo ring; optionally, the ring is substituted with a substituent selected from alkyl, cycloalkyl alkoxy, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂, Se(═O)₂ and N(HNC═X)₂(Ph-halogen(s))₂ wherein X is O or S; also optionally, the ring comprises one or more heteroatom which are the same or different and wherein the heteroatom is selected from O, N, S and Se.
(34) A compound according to (33) having the general formula IIIC′ below

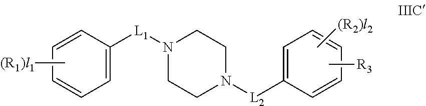

wherein:
R₁, R₂ and R₃ are each independently selected from H, alkyl, cycloalkyl, alkylaryl, alkoxy, thioalkoxy, aryl, aryloxy, thioaryloxy, alkylaryloxy, thioalkylaryloxy, OH, SH, NH₂, a halogen atom, a halogeno alkyl, a halogeno alkoxy, a halogeno thioalkoxy, CN, NO₂, S(═O)₂, Se(═O)₂ and N(HNC═X)₂(Ph-halogen(s))₂ wherein X is O or S; and
I1 is an integer from 0 to 5; and
I2 is an integer from 0 to 4.
(35) A compound according to (34) having the general formula IVC′ below

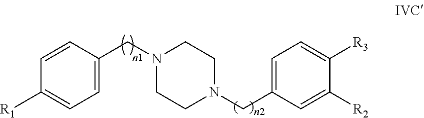

wherein: n1 and n2 are each independently an integer from 0 to 12.
(36) A compound according to (34), wherein L₁ and L₂ are each independently (CH₂)ₙ wherein n is an integer from 0 to 12.
(37) A compound according to (36), wherein n1 and n2 are each independently an integer from 1 to 3.
(38) A compound according to (36), wherein R₁ and R₃ are each independently an alkoxy or a halogen.
(39) A compound according to (36), wherein R₂ is N(HNC═X)₂(Ph-halogen(s))₂ wherein X is O or S.
(40) A compound according to (24), wherein Q is the piperazine ring.
(41) A compound according to (24), which is selected from the group of compounds depicted in the Table 5 herein below.
(42) A compound according to (35), which is compound R28 depicted below

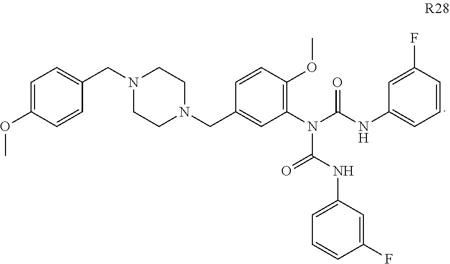 [SMILES: COc1ccc(CN2CCN(Cc3ccc(OC)c(N(C(=O)Nc4cccc(F)c4)C(=O)Nc4cccc(F)c4)c3)CC2)cc1]

(43) A pharmaceutical composition comprising a compound as defined in any one of (1) to (42), and a pharmaceutically acceptable carrier.
(44) A kit comprising a compound as defined in any one of (1) to (42) and/or a pharmaceutical composition as defined in (43), another therapeutic agent, and instructions for use in the treatment of a medical condition involving Ran GTPase.
(45) A kit according to (44), wherein the other therapeutic agent comprises a DNA damaging agent such as carboplatin and/or an inhibitor of poly ADP ribase polymerase (PARP) such as olaparib.
(46) A compound according to any one of (1) to (42), which inhibits Ran GTPase.
(47) A method of treating a medical condition involving Ran GTPase, comprising administering to a subject a therapeutically effective amount of a compound as defined in any one of (1) to (42) or a pharmaceutical composition as defined in (43).
(48) A method of treating a medical condition involving Ran GTPase, comprising administering to a subject a therapeutically effective amount of compound M26, V188, 1292 or a pharmaceutical composition comprising same.
(49) A method according to (47) or (48), wherein the medical condition is a medical condition with immune disorder.
(50) A method according to any one of (47) to (49), wherein the medical condition is cancer including ovarian cancer, breast cancer, pancreatic cancer, colorectal cancer and a cancer embodying aneuploidy.
(51) A method according to any one of (47) to (50), further comprising treating the subject with a second therapy.
(52) A method according to (51), wherein the second therapy comprises a DNA damaging agent such as carboplatin and/or an inhibitor of poly ADP ribase polymerase (PARP) such as olaparib.
(53) A method according to any one of (47) to (51), wherein the compound is administered orally, intravenously, intra-arterially, subcutaneously, topically or intramuscularly.
(54) A method according to (50), wherein the cancer is primary or multi-drug resistant, metastatic and/or recurrent.
(55) A method according to (50) or (54), wherein the method comprises inhibiting cancer growth, killing cancer cells, reducing tumor burden, reducing tumor size, improving the subject's quality of life and/or prolonging the subject's length of life.
(56) A method according to any one of (43) to (50), wherein the subject is human.
(57) A method according to any one of (47) to (56), wherein the subject is a non-human animal.
(58) Use of a compound as defined in any one of (1) to (42) or a pharmaceutical composition as defined in (43), for treating in a subject, a medical condition involving Ran GTPase.
(59) Use of compound M26, V188, 1292 or a pharmaceutical composition comprising same, for treating in a subject, a medical condition involving Ran GTPase.
(60) Use of a compound as defined in any one of (1) to (42), in the manufacture of a medicament for treating a medical condition involving Ran GTPase.
(61) Use of compound M26, V188 or 1292, in the manufacture of a medicament for treating a medical condition involving Ran GTPase.
(62) A compound as defined in any one of (1) to (42), for use in the treatment of a medical condition that involves Ran GTPase.
(63) A pharmaceutical composition as defined in (43), for use in the treatment of a medical condition that involves Ran GTPase.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. In the appended drawings:

FIG. 1: Relation between Ran expression (immunohistochemistry) and cumulative survival of patients with EOC. Kaplan-Meier graphical representation of survival curves demonstrated a poorer survival associated with high expression of Ran. p<0.001 in A), B) and D), and p=0.06 in C). G0-G3 denotes grades 0-3 [27].

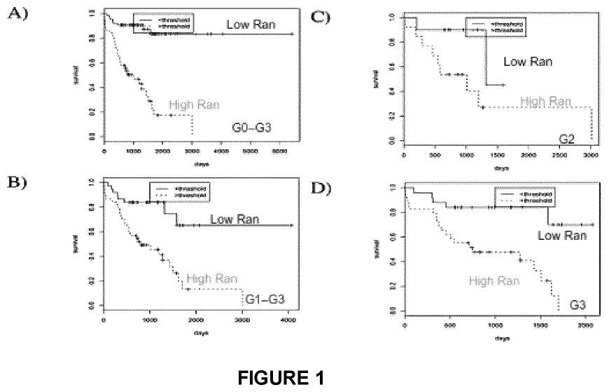

FIG. 2: Loss of Ran expression results in caspase-3 associated apoptosis in EOC cell lines (TOV112D and TOV1946) and tumor regression in vivo (xenograft of TOV112D). P=parental cell line; M.P.=mixed population; #1-3=number of stable clones [25].

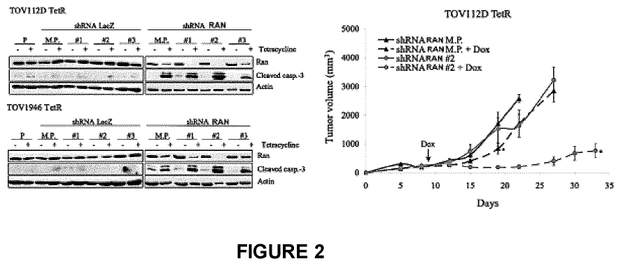

FIG. 3: Effect of Ran knockdown by siRNA in EOC cell lines and normal ARPE-19. A) Cells were transfected with scrambled siRNA (siScr, black bars) or Ran siRNA (siRan, gray bars) and subjected to clonogenic assay. Bars represent percentage of colonies formed compared to siScr for each cell line. B) Cells were transfected with siScr or siRan and seeded on 96-well plates for cell proliferation measurements during 3 days using the live cell imaging IncuCyte system. Each point represents percentage of cell number compared to siScr. C) Apoptosis was evaluated 96 h post-transfection by analysis of PARP cleavage. Levels of Ran expression were evaluated by Western blot, as shown on the upper panels. D) Apoptosis was evaluated 96 h post-transfection by flow cytometry analysis using AnnexinV and DRAQ 7. Bars represent percentage of Annexin V-positive cells in total cells counted per condition in each cell line.

FIG. 4: Sensitivity of ARPE-19 and TOV81D cells to Ran knockdown after polyploidy induction by cytochalasin D. A) Levels of active Ran-GTP were assessed by pull-down assays in dividing cells after synchronization in G2/M phase of the cell cycle by nocodazol treatment. B) Analysis of binucleated cells (polyploidy) after cytochalasin D treatment. Bars represent percentage of cells counted as mono (black bars) or binucleated (white bars) in total cells counted. C) Cell proliferation assay by IncuCyte after transfection with siScr and two different siRan. Each point represents relative cell growth compared to day 0.

FIG. 5: Biological activity of putative Ran inhibitors. A) After virtual inspection of NCI compounds binding to a specific pocket in Ran crystal structure, 45 compounds were selected (in two batches, first 28; second 17) for biological activity testing. Cumulative results of colony formation inhibition by these compounds (10 μM) in normal ARPE-19 and EOC TOV112D cells. Only compounds that inhibited TOV112D without affecting ARPE-19 were chosen for future studies (green arrows). B)-C) Examples of results obtained in each batch. Bars represent percentage of colonies formed compared to DMSO-treated controls.

FIG. 6: Characterization of lead compounds M26 and V188. A) Surface Plasma Resonance (SPR) assays showing binding curves at difference compound concentrations and calculated Kd values. B) Pull-down assays showing decreased active Ran-GTP levels after M26 and V188 treatments. C) Clonogenic assay using different concentrations of V188 compound in ARPE-19 and TOV112D cells. IC50 value was calculated for the TOV112D cells. Bars and curve points represent percentage of colonies formed compared to DMSO-treated controls. D) Cell proliferation assay of different EOC cell lines and the normal ARPE cells using the live cell imaging IncuCyte system and 40 μM V188. Each point represents cell numbers obtained in comparison to DMSO-treated controls. E) Schematic representation of work distribution.

FIG. 7: Characterization of M26 Analogs. A) Clonogenic assay using 10 μM of different compounds in ARPE-19 (white bars) and TOV112D (red bars) cells. Bars represent percentage of colonies formed compared to DMSO-treated controls. B) Detailed clonogenic assay using different concentrations of M36 compound in ARPE-19 and TOV112D cells. IC50 value was calculated for the TOV112D cells. Bars and curve points represent percentage of colonies formed compared to DMSO-treated controls. C) Cell proliferation assay of different EOC cell lines and the normal ARPE cells using the live cell imaging IncuCyte system and 40 μM M36. Each point represents cell numbers obtained in comparison to DMSO-treated controls. D) Apoptosis was evaluated 96 hours after M36 treatment in different cell lines by analysis of PARP cleavage. E) SPR assays showing binding curves at difference M36 concentrations and calculated Kd value. F) Pull-down assays showing decreased active Ran-GTP levels after treatment of TOV112D cells with different concentrations of M36. No decrease in Rac1-GTP levels were observed when pulldown assays of Rac1-GTP was performed with 50 μM M36. G) Immunofluorescence of TOV112D cells treated with M36 (right panel) or DMSO (left panel) using anti-Ran-GTP antibody (pink). Nuclei are shown by Dapi staining (blue).

FIG. 8: Characterization of M36 Analogs. A) Cell proliferation assays of the different analogs (40 μM) on normal ARPE-19 and EOC TOV112D cells using the IncuCyte system. Each point represents percentage of cell numbers obtained in comparison to DMSO-treated controls. B) Clonogenic assay using different concentrations (5-20 μM) of the analogs in ARPE-19 and TOV112D cells. Each point represents percentage of colonies formed compared to DMSOtreated controls. Green squares indicate selected M46 compound. C) Detailed clonogenic assay using different concentrations of M46 compound and the ARPE-19 and TOV112D cells. Bars and curve points represent percentage of colonies formed compared to DMSO-treated controls. IC50 value was calculated for the TOV112D cells. D) Pull-down assays showing decreased active Ran-GTP levels after treatment of TOV112D cells with different concentrations of M46.

FIG. 9: Characterization of V188 Analogs. A) Cell proliferation assays of the different analogs (40 μM) on normal ARPE-19 and EOC TOV112D cells using the IncuCyte system. Each point represents percentage of cell numbers obtained in comparison to DMSO-treated controls. For compounds 1156 and 1157, bars represent percentage of cell numbers obtained at day 4 in comparison to DMSO-treated controls in ARPE-19 (white bars) and TOV112D (red bars) cells. B) Clonogenic assay using different concentrations (5-20 μM) of the analogs in ARPE-19 and TOV112D cells. Each point represents percentage of colonies formed compared to DMSO treated controls. Green squares indicate selected 1292 compound. C) Detailed clonogenic assay using different concentrations of 1292 compound and the ARPE-19 and TOV112D cells. Bars and curve points represent percentage of colonies formed compared to DMSO-treated controls. IC50 value was calculated for the TOV112D cells. D) Pull-down assays showing decreased active Ran-GTP levels after treatment of TOV112D cells with different concentrations of 1292.

FIG. 10: Characterization of 1292 Analogs. A) Cell proliferation assays of the different analogs (80 μM) on normal ARPE-19 and EOC TOV112D cells using the IncuCyte system. Each point represents percentage of cell numbers obtained in comparison to DMSO-treated controls. Green square indicates selected R20 compound. B) Detailed clonogenic assay using different concentrations of R20 compound and the ARPE-19 and TOV112D cells. Bars and curve points represent percentage of colonies formed compared to DMSO-treated controls. IC50 value was calculated for the TOV112D cells. C) Pull-down assays showing decreased active Ran-GTP levels after treatment of TOV112D cells with different concentrations of R20.

FIG. 11: Further analysis of compounds 1292 and R20. (A) and C)) Cell proliferation assay of different EOC cell lines and the normal ARPE cells using the live cell imaging IncuCyte system and 80 μM of 1292 or R20. Each point represents cell numbers obtained in comparison to DMSO-treated controls. B) Apoptosis was evaluated 96 hours after 1292 treatment in different cell lines by analysis of PARP cleavage.

FIG. 12: Effects of different R20 salts on cell growth. Cell proliferation assays of the different salt formulations of compound R20 on normal ARPE-19 and EOC TOV112D cells using the IncuCyte system and different concentrations (20, 40, 80 μM) of the analogs. Each point represents the ratio of cell number relative day zero.

FIG. 13: Pharmacokinetics and tolerance studies of M36 and QR20 compounds. A) Measurements of plasma levels of the compounds after a single intravenous or intraperitoneal injection in CD1 mice. Mice were sacrificed 5 minutes, 15 minutes, 30 minutes, 1 hour, 2 hours and 6 hours after injections of M36 or QR20 (50 mg/kg). B) M36 or QR20 compounds were i.p. injected daily on NRG mice for 15 days at 75 mg/kg. For controls, mice were i.p. injected with vehicle (DMSO 10%, Kolliphor® EL 10%, PEG-400 20% and PBS 60%) under the same conditions. Mouse weight was measured daily (except week-ends) and plotted as percentage in comparison to their weight on the day of first injection.

FIG. 14: Characterization of new M36 Analogs. A) Cell proliferation assays of the different analogs (40 μM) on normal ARPE-19 and EOC TOV112D cells using the IncuCyte system. Bars represent percentage of cell numbers obtained in comparison to DMSO-treated controls on day 4. Green box indicates selected M55 compound. B) Detailed cell proliferation assays using different concentrations (5, 10, 20 μM) of compound M55 on normal ARPE-19 and EOC TOV112D cells. Each point represents the ratio of cell number relative day zero. C) Detailed clonogenic assay using different concentrations of compound M55 and the ARPE-19 and TOV112D cells. Bars and curve points represent percentage of colonies formed compared to DMSO-treated controls. IC50 value was calculated for the TOV112D cells.

FIG. 15: Further characterization of new M36 Analogs. A) Cell proliferation assays using different concentrations (0.5, 1, 2.5 μM) of compounds M48, M51 and M52 on normal ARPE-19 and EOC TOV112D cells. Each point represents the ratio of cell number relative day zero. B) Detailed clonogenic assay using different concentrations of compounds M48, M51 and M52 and the ARPE-19 and TOV112D cells. Bars and curve points represent percentage of colonies formed compared to DMSO-treated controls. IC50 value was calculated for the TOV112D cells.

FIG. 16: Characterization of R20 Analogs. A) Cell proliferation assays of the different compounds at 20, 40 or 80 μM concentrations on normal ARPE-19 (white bars) and EOC TOV112D (red bars) cells using the IncuCyte system. Bars represent percentage of cell numbers obtained in comparison to DMSO-treated controls on day 4. Green underlines indicate selected R28 compound. B) Detailed clonogenic assay using different concentrations of R28 compound and the ARPE-19 and TOV112D cells. Bars and curve points represent percentage of colonies formed compared to DMSO-treated controls. IC50 value was calculated for the TOV112D cells. C) Immunofluorescence of TOV112D cells treated with R28 (right panel) or DMSO (left panel) using anti-Ran-GTP antibody (pink). Nuclei are shown by Dapi staining (blue).

FIG. 17: Effect of selected compounds (M36, QR20, R28, M55, M51) on cell lines of other cancer types. A) Prostate cancer cell lines: Hormone sensitive, LNCaP and 22RV1; and Castrate-resistant, PC3 and DU145. B) Triple negative breast cancer cell lines, MDA231, MDA468 and HCC1806. C) Other types of breast cancer cell lines: Luminal A (ER+Her2-), ZR75 and T47D; Luminal B (ER+Her2+), BT474; and Her2+, HCC1954. Bars represent the ratio of cell number relative day zero. DMSO=vehicle control.

FIG. 18: Summary of screening and selection of compounds. Orange boxes denote compounds with good biological activity and known chemical structures. Red boxes denote compounds with good biological activity and novel chemical structures.

FIG. 19: Involvement of Ran (and its inhibitor M36) in DNA damage. (A) Representative images of γH2AX (red) foci in normal diploid ARPE, as well as diploid TOV81D and three aneuploid EOC cells after Ran knockdown. (B-C) Quantitative analysis of γH2AX foci in normal and EOC cells (B), and after Ran knockdown (C). TOV112D cells were treated with M36 compound for 48 hours or 72 hours and then immunostained for γH2AX (D). TOV112D cells were transfected with siRan (E) or treated with M36 (F) compound and then exposed to gamma-irradiation. Cells were fixed at the indicated recovery time points and immunostained for γH2AX. Quantitative analysis of γH2AX foci are shown. *<0.05**<0.01.

FIG. 20: Role of Ran and impact of M36 on double-strand DNA damage repair. (A and C) Representative images (right panels) and quantification (left panels) of immunofluorescent staining of RAD51/Geminin (A) or 53BP1 (C) positive nuclei in irradiated Ran KD or control TOV112D cells. (B and D) TOV112D cells were treated with M36 compound followed by gamma-irradiation. Cells were then subjected to RAD51/Gemini (B) and 53BP1 (D) immunostaining and foci were quantified. For each condition, RAD51/Gemini or 53BP1 foci were counted in at least 1000 nuclei using Axiovision software. Results were normalized with the number of foci in the corresponding non-irradiated cells. (E-F) TOV112D cells were treated with siRan (E) or compound M36 (F) and 53BP1 immunofluorescence was performed as in C and D. Then cytoplasmically labeled cells were visualized by microscopy and quantified. The presence of cytoplasmic staining of 53BP1 was analyzed in at least 100 cells. *=p<0.05.

FIG. 21: Specificity of compound M36. (A) Activation ELISA assays for RhoA and Cdc42 were conducted in TOV112D cells treated with M36 or DMSO. No decrease in RhoA- or Cdc42-GTP levels were observed. (B) TOV112D cells were transfected with plasmid containing Ran wild type (VVT) or dominant active (DA) mutant fused with GFP, or with empty plasmid. Left panel shows levels of expressed proteins analyzed by western blot; actin served as a loading control. Right panel shows cell survival curves for transfected cells after treatment with compound M36.

FIG. 22: Characterization of new synthesized compounds. Cell proliferation assays of the different compounds at different concentrations were performed on normal ARPE-19 (white bars) and EOC TOV112D (red bars) cells using the IncuCyte system. Bars represent percentage of cell numbers obtained in comparison to DMSO-treated controls on day 4. Green boxes indicate selected M66, M88 and M93 compounds.

### DESCRIPTION OF ILLUSTRATIVE EMBODIMENTS

Before the present invention is further described, it is to be understood that the invention is not limited to the particular embodiments described below, as variations of these embodiments may be made and still fall within the scope of the appended claims. It is also to be understood that the terminology employed is for the purpose of describing particular embodiments, and is not intended to be limiting. Instead, the scope of the present invention will be established by the appended claims.

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

As used herein, the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “alkyl” or “alk” as used herein, represents a monovalent group derived from a straight or branched chain saturated hydrocarbon comprising, unless otherwise specified, from 1 to 15 carbon atoms and is exemplified by methyl, ethyl, n- and iso-propyl, n-, sec-, iso- and tert-butyl, neopentyl and the like and may be optionally substituted with one, two, three or, in the case of alkyl groups comprising two carbons or more, four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group comprises one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbon atoms; (16) N-protected amino; (17) nitro; (18) oxo or thiooxo; (19) perfluoroalkyl of 1 to 4 carbon atoms; (20) perfluoroalkoxyl of 1 to 4 carbon atoms; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group comprises one to six carbon atoms, (d) substituted or unsubstituted C₁₋₉ heterocyclyl, and (e) substituted or unsubstituted C₂₋₁₅ heterocyclylalkyl, where the alkylene group comprises one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (27) C(O)NR^{C}R^{D}, where each of R^{C} and R^{D} is independently selected from the group consisting of (a) hydrogen, (b) alkyl, (c) aryl and (d) arylalkyl, where the alkylene group comprises one to six carbon atoms; (28) S(O)R^{E}, where R^{E} is selected from the group consisting of (a) alkyl, (b) aryl, (c) arylalkyl, where the alkylene group comprises one to six carbon atoms, and (d) hydroxyl; (29) S(O)₂R^{E}, where R^{E} is selected from the group consisting of (a) alkyl, (b) aryl, (c) arylalkyl, where the alkylene group comprises one to six carbon atoms, and (d) hydroxyl; (30) S(O)₂NR^{F}R^{G}, where each of R^{F} and R^{G} is independently selected from the group consisting of (a) hydrogen, (b) alkyl, (c) aryl and (d) arylalkyl, where the alkylene group comprises one to six carbon atoms; and (31) —NR^{H}R^{I}, where each of R^{H} and R^{I} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms, (i) alkcycloalkyl, where the cycloalkyl group comprises three to eight carbon atoms, and the alkylene group comprises one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The terms “alkoxy” or “alkyloxy” as used interchangeably herein, represent an alkyl group attached to the parent molecular group through an oxygen atom.

The term “alkylsulfonyl” as used herein, represents an alkyl group attached to the parent molecular group through a S(O)₂ group.

The term “alkylthio” as used herein, represents an alkyl group attached to the parent molecular group through a sulfur atom.

The term “alkylene” as used herein, represents a saturated divalent hydrocarbon group derived from a straight or branched chain saturated hydrocarbon by the removal of two hydrogen atoms, and is exemplified by methylene, ethylene, isopropylene and the like.

The term “alkenyl” as used herein, represents monovalent straight or branched chain groups of, unless otherwise specified, from 2 to 15 carbons, such as, for example, 2 to 6 carbon atoms or 2 to 4 carbon atoms, containing one or more carbon-carbon double bonds and is exemplified by ethenyl, 1-propenyl, 2-propenyl, 2-methyl-1-propenyl, 1-butenyl, 2-butenyl and the like and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group comprises one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbon atoms; (16) N-protected amino; (17) nitro; (18) oxo or thiooxo; (19) perfluoroalkyl of 1 to 4 carbon atoms; (20) perfluoroalkoxyl of 1 to 4 carbon atoms; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group comprises one to six carbon atoms, (d) substituted or unsubstituted C₁₋₉ heterocyclyl, and (e) substituted or unsubstituted C₂₋₁₅ heterocyclylalkyl, where the alkylene group comprises one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (27) C(O)NR^{C}R^{D}, where each of R^{C} and R^{D} is independently selected from the group consisting of (a) hydrogen, (b) alkyl, (c) aryl and (d) arylalkyl, where the alkylene group comprises one to six carbon atoms; (28) S(O)R^{E}, where R^{E} is selected from the group consisting of (a) alkyl, (b) aryl, (c) arylalkyl, where the alkylene group comprises one to six carbon atoms, and (d) hydroxyl; (29) S(O)₂R^{E}, where R^{E} is selected from the group consisting of (a) alkyl, (b) aryl, (c) arylalkyl, where the alkylene group comprises one to six carbon atoms, and (d) hydroxyl; (30) S(O)₂NR^{F}R^{G}, where each of R^{F} and R^{G} is independently selected from the group consisting of (a) hydrogen, (b) alkyl, (c) aryl and (d) arylalkyl, where the alkylene group comprises one to six carbon atoms; and (31) —NR^{H}R^{I}, where each of R^{H} and R^{I} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms; (i) alkcycloalkyl, where the cycloalkyl group comprises three to eight carbon atoms, and the alkylene group comprises one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The term “alkynyl” as used herein, represents monovalent straight or branched chain groups of from two to six carbon atoms comprising a carbon-carbon triple bond and is exemplified by ethynyl, 1-propynyl, and the like and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group comprises one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbon atoms; (16) N-protected amino; (17) nitro; (18) oxo or thiooxo; (19) perfluoroalkyl of 1 to 4 carbon atoms; (20) perfluoroalkoxyl of 1 to 4 carbon atoms; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group comprises one to six carbon atoms, (d) substituted or unsubstituted C₁₋₉ heterocyclyl, and (e) substituted or unsubstituted C₂₋₁₅ heterocyclylalkyl, where the alkylene group comprises one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (27) C(O)NR^{C}R^{D}, where each of R^{C} and R^{D} is independently selected from the group consisting of (a) hydrogen, (b) alkyl, (c) aryl and (d) arylalkyl, where the alkylene group comprises one to six carbon atoms; (28) S(O)R^{E}, where R^{E} is selected from the group consisting of (a) alkyl, (b) aryl, (c) arylalkyl, where the alkylene group comprises one to six carbon atoms, and (d) hydroxyl; (29) S(O)₂R^{E}, where R^{E} is selected from the group consisting of (a) alkyl, (b) aryl, (c) arylalkyl, where the alkylene group comprises one to six carbon atoms, and (d) hydroxyl; (30) S(O)₂NR^{F}R^{G}, where each of R^{F} and R^{G} is independently selected from the group consisting of (a) hydrogen, (b) alkyl, (c) aryl and (d) arylalkyl, where the alkylene group comprises one to six carbon atoms; and (31) —NR^{H}R^{I}, where each of R^{H} and R^{I} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms, (i) alkcycloalkyl, where the cycloalkyl group comprises three to eight carbon atoms, and the alkylene group comprises one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The term “aryl” as used herein, represents mono- and/or bicyclic carbocyclic ring systems and/or multiple rings fused together and is exemplified by phenyl, naphthyl, 1,2-dihydronaphthyl, 1,2,3,4-tetrahydronaphthyl, fluorenyl, indanyl, indenyl and the like and may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups independently comprise from one to six carbon atoms; (5) alkylsulfinyl of one to six carbon atoms; (6) alkylsulfinylalkyl, where the alkyl and alkylene groups independently comprise from one to six carbon atoms; (7) alkylsulfonyl of one to six carbon atoms; (8) alkylsulfonylalkyl, where the alkyl and alkylene groups are independently comprised of one to six carbon atoms; (9) aryl; (10) arylalkyl, where the alkyl group comprises one to six carbon atoms; (11) amino; (12) aminoalkyl of one to six carbon atoms; (13) aryl; (14) arylalkyl, where the alkylene group comprises one to six carbon atoms; (15) aryloyl; (16) azido; (17) azidoalkyl of one to six carbon atoms; (18) carboxaldehyde; (19) (carboxaldehyde)alkyl, where the alkylene group comprises one to six carbon atoms; (20) cycloalkyl of three to eight carbon atoms; (21) alkcycloalkyl, where the cycloalkyl group comprises three to eight carbon atoms and the alkylene group comprises one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group comprises one to six carbon atoms; (33) oxo; (34) thioalkoxy of one to six carbon atoms; (35) thioalkoxyalkyl, where the alkyl and alkylene groups independently comprise from one to six carbon atoms; (36) (CH₂)_{q}CO₂R^{A}, where q is an integer ranging from zero to four and R^{A} is selected from the group consisting of (a) alkyl, (b) aryl, and (c) arylalkyl, where the alkylene group comprises one to six carbon atoms; (37) (CH₂)_{q}C(O)NR^{B}R^{C}, where R^{B} and R^{C} are independently selected from the group consisting of (a) hydrogen, (b) alkyl, (c) aryl, and (d) arylalkyl, where the alkylene group comprises one to six carbon atoms; (38) (CH₂)_{q}S(O)₂R^{D}, where R^{D} is selected from the group consisting of (a) alkyl, (b) aryl, and (c) arylalkyl, where the alkylene group comprises one to six carbon atoms; (39) (CH₂)_{q}S(O)₂NR^{E}R^{F}, where each of R^{E} and R^{F} is independently selected from the group consisting of (a) hydrogen, (b) alkyl, (c) aryl, and (d) arylalkyl, where the alkylene group comprises one to six carbon atoms; (40) (CH₂)_{q}NR^{G}R^{H}, where each of R^{G} and R^{H} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms, and (i) alkcycloalkyl, where the cycloalkyl group comprises three to eight carbon atoms, and the alkylene group comprises one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “alkaryl” represents an aryl group attached to the parent molecular group through an alkyl group.

The term “aryloxy” as used herein, represents an aryl group that is attached to the parent molecular group through an oxygen atom.

The term “cycloalkyl” as used herein, represents a monovalent saturated or unsaturated non-aromatic cyclic hydrocarbon group of three to eight carbon atoms, unless otherwise specified, and is exemplified by cyclopropyl, cyclobutyl, cyclopentyl, cyclohexyl, cycloheptyl, bicyclo[2.2.1]heptyl and the like. The cycloalkyl groups of the present disclosure can be optionally substituted with: (1) alkanoyl of one to six carbon atoms; (2) alkyl of one to six carbon atoms; (3) alkoxy of one to six carbon atoms; (4) alkoxyalkyl, where the alkyl and alkylene groups independently comprise from one to six carbon atoms; (5) alkylsulfinyl of one to six carbon atoms; (6) alkylsulfinylalkyl, where the alkyl and alkylene groups independently comprise from one to six carbon atoms; (7) alkylsulfonyl of one to six carbon atoms; (8) alkylsulfonylalkyl, where the alkyl and alkylene groups independently comprise from one to six carbon atoms; (9) aryl; (10) arylalkyl, where the alkyl group comprises one to six carbon atoms; (11) amino; (12) aminoalkyl of one to six carbon atoms; (13) aryl; (14) arylalkyl, where the alkylene group comprises one to six carbon atoms; (15) aryloyl; (16) azido; (17) azidoalkyl of one to six carbon atoms; (18) carboxaldehyde; (19) (carboxaldehyde)alkyl, where the alkylene group comprises one to six carbon atoms; (20) cycloalkyl of three to eight carbon atoms; (21) alkcycloalkyl, where the cycloalkyl group comprises three to eight carbon atoms and the alkylene group comprises one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group comprises one to six carbon atoms; (33) oxo; (34) thioalkoxy of one to six carbon atoms; (35) thioalkoxyalkyl, where the alkyl and alkylene groups independently comprise from one to six carbon atoms; (36) (CH₂)_{q}CO₂R^{A}, where q is an integer ranging from zero to four and R^{A} is selected from the group consisting of (a) alkyl, (b) aryl, and (c) arylalkyl, where the alkylene group comprises one to six carbon atoms; (37) (CH₂)_{q}C(O)NR^{B}R^{C}, where each of R^{B} and R^{C} is independently selected from the group consisting of (a) hydrogen, (b) alkyl, (c) aryl, and (d) arylalkyl, where the alkylene group comprises one to six carbon atoms; (38) (CH₂)_{q}S(O)₂R^{D}, where R^{D} is selected from the group consisting of (a) alkyl, (b) aryl, and (c) arylalkyl, where the alkylene group comprises one to six carbon atoms; (39) (CH₂)_{q}S(O)₂NR^{E}R^{F}, where each of R^{E} and R^{F} is independently, selected from the group consisting of (a) hydrogen, (b) alkyl, (c) aryl, and (d) arylalkyl, where the alkylene group comprises one to six carbon atoms; (40) (CH₂)_{q}NR^{G}R^{H}, where each of R^{G} and R^{H} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms and (i) alkcycloalkyl, where the cycloalkyl group comprises three to eight carbon atoms, and the alkylene group comprises one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “halogen” or “halo” as used interchangeably herein, represents F, Cl, Br and I.

The term “heteroaryl” as used herein, represents that subset of heterocycles, as defined herein, which is aromatic: (i.e., containing 4n+2 pi electrons within a mono- or multicyclic ring system).

The terms “heterocycle” or “heterocyclyl” as used interchangeably herein represent a 5-, 6- or 7-membered ring, unless otherwise specified, comprising one, two, three, or four heteroatoms independently selected from the group consisting of nitrogen, oxygen, and sulfur. The 5-membered ring has from zero to two double bonds and the 6- and 7-membered rings have from zero to three double bonds. The term “heterocycle” also includes bicyclic, tricyclic, and tetracyclic groups in which any of the above heterocyclic rings is fused to one or two rings independently selected from the group consisting of an aryl ring, a cyclohexane ring, a cyclohexene ring, a cyclopentane ring, a cyclopentene ring and another monocyclic heterocyclic ring such as indolyl, quinolyl, isoquinolyl, tetrahydroquinolyl, benzofuryl, benzothienyl and the like. Heterocycles include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroinidolyl, tetrahydroquinolyl, tetrahydroisoquinolyl, pyranyl, dihydropyranyl, dithiazolyl, benzofuranyl, benzothienyl and the like. Heterocyclic groups also include compounds of the formula

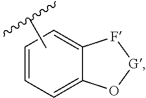

where F′ is selected from the group consisting of CH₂, CH₂O and O, and G′ is selected from the group consisting of C(O) and (C(R′)(R″))ᵥ, where each of R′ and R″ is independently select from the group consisting of hydrogen and alkyl of one to four carbon atoms, and v is an integer ranging from one to three, and includes groups such as 1,3-benzodioxolyl, 1,4-benzodioxanyl and the like. Any of the heterocyclic groups mentioned herein may be optionally substituted with one, two, three, four or five substituents independently selected from the group consisting of: (1) alkanoyl of one to six carbon atoms; (2) alkyl of one to six carbon atoms; (3) alkoxy one to six carbon atoms; (4) alkoxyalkyl, where the alkyl and alkylene group independently comprise from one to six carbon atoms; (5) alkylsulfinyl of one to six carbon atoms; (6) alkylsulfinylalkyl, where the alkyl and alkylene groups independently comprise from one to six carbon atoms; (7) alkyrsulfonyl of one to six carbon atoms; (8) alkylsulfonylalkyl, where the alkyl and alkylene groups independently comprise from one to six carbon atoms; (9) aryl; (10) arylalkyl, where the alkyl group comprises one to six carbon atoms; (11) amino; (12) aminoalkyl of one to six carbon atoms; (13) aryl; (14) arylalkyl, where the alkylene group comprises one to six carbon atoms; (15) aryloyl; (16) azido; (17) azidoalkyl of one to six carbon atoms; (18) carboxaldehyde; (19) (carboxaldehyde)alkyl, where the alkylene group comprises one to six carbon atoms; (20) cycloalkyl of three to eight carbon atoms; (21) alkcycloalkyl, where the cycloalkyl group comprises from three to eight carbon atoms and the alkylene group comprises from one to ten carbon atoms; (22) halo; (23) haloalkyl of one to six carbon atoms; (24) heterocycle; (25) (heterocycle)oxy; (26) (heterocycle)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group comprises from one to six carbon atoms; (33) oxo; (34) thioalkoxy of one to six carbon atoms; (35) thioalkoxyalkyl, where the alkyl and alkylene groups independently comprise from one to six carbon atoms; (36) (CH₂)_{q}CO₂R^{A}, where q is an integer ranging from zero to four a R^{A} is selected from the group consisting of (a) alkyl, (b) aryl, and (c) arylalkyl where the alkylene group comprises from one to six carbon atoms; (37) (CH₂)_{q}C(O)NR^{B}R^{C}, where each of R^{B} and R^{C} is independently selected from the group consisting of (a) hydrogen, (b) alkyl, (c) aryl, and (d) arylalkyl, where the alkylene group comprises from one to six carbon atoms; (38) (CH₂)_{q}S(O)₂R^{D}, where R^{D} is selected from the group consisting of (a) alkyl, (b) aryl, and (c) arylalkyl, where the alkylene group comprises from one to six carbon atoms; (39) (CH₂)_{q}S(O)₂NR^{E}R^{F}, where each of R^{E} and R^{F} is independently selected from the group consisting of (a) hydrogen, (b) alkyl, (c) aryl, and (d) arylalkyl, where the alkylene group comprises from one to six carbon atoms; (40) (CH₂)_{q}NR^{G}R^{H} where each of R^{G} and R^{H} is independently selected from the group consisting of (a) hydrogen; (b) an N-protecting group; (c) alkyl of one to six carbon atoms, (d) alkenyl of two to six carbon atoms; (e) alkynyl of two to six carbon atoms; (f) aryl; (g) arylalkyl, where the alkylene group comprises from one to six carbon atoms; (h) cycloalkyl of three to eight carbon atoms, and (i) alkcycloalkyl, where the cycloalkyl group comprises from three to eight carbon atoms and the alkylene group comprises from one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “heteroatom” as used herein, is understood as being oxygen, sulfur, nitrogen or selenium.

The term “thioalkoxy” as used herein, represents an alkyl group attached to the parent molecular group through a sulfur atom. Exemplary unsubstituted thioalkoxy groups comprise from 1 to 6 carbon atoms.

The term “thiocarbonyl” as used herein, represents a C(S) group, which can also be represented as C═S.

The term “salt(s)” as used herein, is understood as being acidic and/or basic salts formed with inorganic and/or organic acids or bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps.

The term “patient as used herein, is understood as being any individual treated with the compounds of the present disclosure.

As used herein the term “therapeutically effective amount” of a compound means an amount sufficient to cure, alleviate or partially arrest the clinical manifestations of a given disease and its complications in a therapeutic intervention comprising the administration of said compound. An amount adequate to accomplish this is defined as “a therapeutically effective amount”. Effective amounts for each purpose will depend on the severity of the disease or injury as well as the weight and general state of the patient.

As used herein the terms “treatment” and “treating” mean the management and care of a patient for the purpose of combating a condition, such as a disease or disorder. The term is intended to include the full spectrum of treatments for a given condition from which the patient is suffering, such administration of the active compounds to alleviate the symptoms or complications, to delay the progression of the condition, and/or to cure or eliminate the condition. The patient to be treated is preferably a mammal, in particular a human being.

The inventors have designed and prepared novel chemical compounds that are small molecules. The compounds according to the invention inhibit Ran GTPase and may be used in the treatment of medical conditions involving Ran GTPase. Such medical conditions may be for example cancers including ovarian cancer, breast cancer, pancreatic cancer, colorectal cancer and cancers embodying aneuploidy.

More specifically, the inventors have investigated the therapeutic value of the compounds according to the invention using in vitro and in vivo epithelial ovarian cancer (EOC) models that they have designed.

Also, the compounds according to the invention may be used in association with other therapeutic agents, which may be for example, DNA damaging agents such as carboplatin, inhibitors of poly ADP ribase polymerase (PARP) such as olaparib.

The present invention is illustrated in further details by the following non-limiting examples.

### CHEMICAL SYNTHESES

Compounds according to the invention have a general formula IA, IIA, IIIA, IB, IIB, IIIB, IIB′, IIIB′, IC, IIC, IIIC, IIC′, IIIC, or IVC′ as illustrated in FIGS. 24-28.

### Example 1—Preparation of M26 and M26 Analogues of Class A

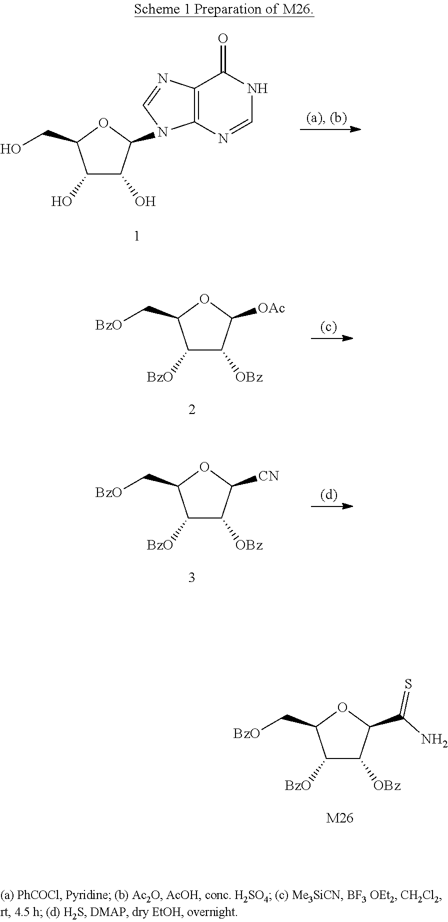

Compound M26 can be prepared by typical methods as illustrated in Scheme 1. The intermediates 2 and 3 were prepared according to the literatures from commercially available inosine 1 [56,57]. Treatment of cyanide 3 by hydrogen sulfide gas and N,N-dimethylaminopyridine in dry EtOH, M26 was obtained.

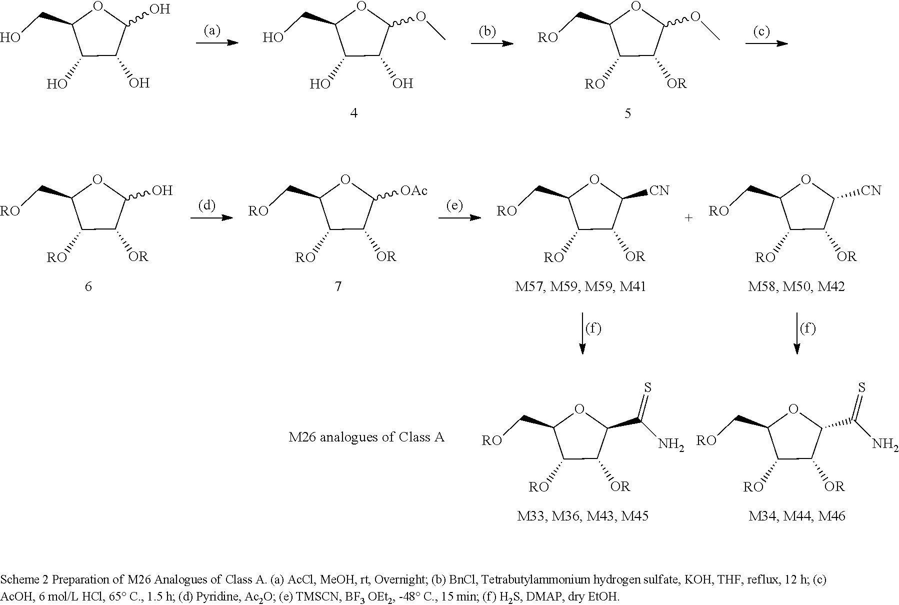

Compounds of M26 Analogues of Class A can be prepared by typical methods as illustrated in Scheme 2. The intermediates 4, 5, 6, 7 were prepared according to the literature from ribose [58]. Compounds 7 were subsequently treated with TMSCN/BF₃.OEt₂ to give the desired cyanide compounds M57˜M59, M39˜M42, which were easily separated by column chromatography. Treatment of those cyanide compounds by hydrogen sulfide gas and N,N-dimethylaminopyridine in dry EtOH gave compounds of M26 Analogues of Class A: M33, M34, M36, M43˜M46.

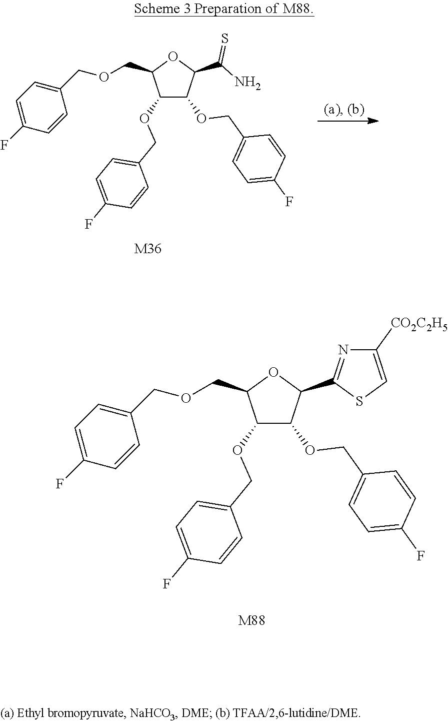

M88 was obtained by the treatment of M36 with ethylbromopyruvate and NaHCO₃ in dry DME, and then by addition of a mixture of trifluoroacetic anhydride and 2,6-lutidine in dry 1,2-dimethoxyethane.

Procedure for the Preparation of M26:

To a suspension of cyanide 3 (0.27 g, 0.6 mmol) in dry EtOH (900 mL, N,N-dimethylaminopyridine (78 mg, 0.06 mmol) was added in one portion under N2. Hydrogen sulfide was slowly passed through the reaction mixture at 0° C. for 2 hours. Then the flask was sealed and stirring continued at room temperature for 16 hours. The reaction was concentrated and purified by column chromatography, M26 was obtained as white solid. Yield 88.2%. 8.51 (br, 1H), 8.12-8.03 (m, 4H), 7.93-7.85 (m, 2H), 7.63-7.42 (m, 8H), 7.34 (t, J=7.8 Hz, 2H), 5.99 (t, J=4.9 Hz, 1H), 5.71 (t, J=5.4 Hz, 1H), 5.12 (d, J=4.6 Hz, 1H), 4.80-4.73 (m, 2H), 4.73-4.67 (m, 1H).

General Procedure for the Preparation of M57˜M59, M47˜M49:

To a solution of 7 (2.1 mmol) in acetonitrile (9 mL), TMSCN (0.47 m1, 3.4 mmol) and BF₃.OEt₂ (0.34 mL, 2.7 mmol) were added dropwise after the solution was cooled to −48° C. The resulting mixture was stirred for 15 minutes at the same temperature. Then the reaction was quenched by addition of saturated aq. ammonium chloride. The resulting mixture extracted with CH₂Cl₂. The combined organic layers were dried over anhydrous Na₂SO₄, filtered and concentrated under reduced pressure. The residue was purified by column chromatography to yield M57˜M59, M47˜M49.

General Procedure for the Preparation of M33, M34, M36, M39˜M42:

To a suspension of cyanide (0.6 mmol) in dry EtOH (20 mL), N,N-dimethylaminopyridine (78 mg, 0.06 mmol) was added in one portion under N₂. Hydrogen sulfide was slowly passed through the reaction mixture at 0° C. for 2 hours. Then the flask was sealed and stirring continued at room temperature for 16 hours. The reaction was concentrated and purified by column chromatography, M33, M33, M34, M36, M39˜M42 were obtained.

Procedure for the Preparation of M88:

Ethyl bromopyruvate (0.2 g, 1 mmol) was add dropwise to a stirred mixture of M36 (0.26 g, 0.5 mmol) and NaHCO₃, (0.42 g, 5 mmol) in dry 1,2-dimethoxyethane (10 mL) at 0° C. under argon atmosphere. Then the reaction mixture was stirred at 0° C. under argon for 6 hours. The reaction was cooled to −15° C. under argon. A solution of trifluoroacetic anhydride (0.32 g, 1.5 mmol) and 2,6-lutidine (12.8 g, 120 mmol) in dry 1,2-dimethoxyethane (20 mL) was added dropwise. Then the reaction mixture was stirred at −15° C. for 2 hours under an argon atmosphere. Water was added to quench the reaction and extracted with CH₂Cl₂ and washed with saturated NaHCO₃ solution. The organic layer was dried over anhydrous Na₂SO₄, filtered, and concentrated. The crude residue was purified by column chromatography to give M88 as colorless syrup. Yield: 87.9%. ¹H NMR (400 MHz, CDCl₃) δ 9.24 (s, 1H), 7.56-7.52 (m, 2H), 7.24-7.20 (m, 5H), 7.14-7.10 (m, 6H), 4.84 (s, 1H), 4.92-4.88 (m, 1H), 4.69-4.67 (m, 1H), 4.48 (d, J=11.0 Hz, 1H), 4.40-4.36 (m, 2H), 4.32-4.30 (m, 1H), 4.34-4.29 (m, 3H), 4.13 (d, J=11.6 Hz, 1H), 3.94-3.89 (m, 2H), 3.60 (d, J=10.5 Hz, 1H), 1.4 (t, J=11.0 Hz, 2H).

Characterization of M26 and M26 Analogues of Class A:

M33: White solid. Yield 92%. ¹H NMR (500 MHz, CDCl₃) δ 9.10 (br, 1H), 7.56-7.44 (m, 2H), 7.38-7.26 (m, 9H), 7.25-7.20 (m, 2H), 7.17-7.13 (m, 2H), 7.09 (br, 1H), 4.96 (s, 1H), 4.91 (d, J=12.1 Hz, 1H), 4.71 (d, J=12.1 Hz, 1H), 4.50-4.45 (m, 2H), 4.38 (d, J=10.9 Hz, 1H), 4.35-4.34 (m, 1H), 4.30 (d, J=4.5 Hz, 1H), 4.16 (d, J=11.8 Hz, 1H), 4.00-3.90 (m, 2H), 3.63 (d, J=10.5 Hz, 1H), 1.29-1.23 (m, 1H).

M34: Colorless syrup. Yield 93%. ¹H NMR (500 MHz, CDCl₃) δ 8.13 (br, 1H), 7.59 (br, 1H), 7.39-7.22 (m, 15H), 4.92 (d, J=3.0 Hz, 1H), 4.77 (dd, J=25.8, 11.1 Hz, 2H), 4.58-4.43 (m, 4H), 4.39-4.33 (m, 2H), 4.12 (dd, J=8.9, 3.6 Hz, 1H), 3.71-3.68 (m, 1H), 3.53-3.50 (m, 1H).

M36: White solid. Yield 83%. ¹H NMR (500 MHz, CDCl₃) δ 9.01 (br, 1H), 7.51-7.41 (m, 2H), 7.18-7.13 (m, 5H), 7.06-6.96 (m, 6H), 4.94 (s, 1H), 4.86 (d, J=12.1 Hz, 1H), 4.67 (d, J=12.1 Hz, 1H), 4.48 (d, J=11.0 Hz, 1H), 4.40-4.36 (m, 2H), 4.32-4.30 (m, 1H), 4.28 (d, J=4.6 Hz, 1H), 4.13 (d, J=11.6 Hz, 1H), 3.94-3.89 (m, 2H), 3.60 (d, J=10.5 Hz, 1H). HRMS (ESI) m/z Found: 540.14650 [M+H]⁺, Calcd: 540.14268.

M39: Colorless syrup. Yield 42%. ¹H NMR (500 MHz, CDCl₃) δ 7.37-7.26 (m, 3H), 7.13-6.95 (m, 9H), 4.72-4.46 (m, 7H), 4.35-4.33 (m, 1H), 4.27-4.25 (m, 1H), 4.12-4.10 (m, 1H), 3.62-3.54 (m, 2H).

M40: Colorless syrup. Yield 28%. ¹H NMR (500 MHz, CDCl₃) δ 7.37-7.26 (m, 3H), 7.19-6.90 (m, 9H), 4.82 (d, J=6.0 Hz, 1H), 4.75-4.66 (m, 3H), 4.60-4.41 (m, 4H), 4.37-4.35 (m, 1H), 4.22-4.15 (m, 1H), 4.03-4.01 (m, 1H), 3.59-3.50 (m, 2H).

M41: White solid. Yield 41%. ¹H NMR (500 MHz, CDCl₃) δ 7.52-7.45 (m, 2H), 7.37-7.22 (m, 4H), 7.19-6.96 (m, 6H), 4.84 (d, J=6.1 Hz, 1H), 4.78 (s, 2H), 4.77-4.62 (m, 3H), 4.62-4.49 (m, 2H), 4.35-4.33 (m, 1H), 4.29-4.22 (m, 1H), 4.07 (t, J=4.5 Hz, 1H), 3.67-3.51 (m, 2H).

M42: Colorless syrup. Yield 25%. ¹H NMR (500 MHz, CDCl₃) δ 7.44-7.26 (m, 6H), 7.16-6.99 (m, 6H), 4.71 (s, 2H), 4.67-4.57 (m, 6H), 4.39-4.37 (m, 1H), 4.25-4.23 (m, 1H), 4.14-4.12 (m, 1H), 3.67-3.56 (m, 2H).

M43: White solid. Yield 82%. ¹H NMR (500 MHz, CDCl₃) δ 8.97 (br, 1H), 7.36-7.15 (m, 6H), 7.05-6.91 (m, 5H), 6.91-6.84 (m, 1H), 4.95 (s, 1H), 4.90 (d, J=12.4 Hz, 1H), 4.70 (d, J=12.4 Hz, 1H), 4.53-4.42 (m, 3H), 4.36-4.35 (m, 1H), 4.31 (d, J=4.6 Hz, 1H), 4.23 (d, J=12.1 Hz, 1H), 4.03-3.92 (m, 2H), 3.67-3.65 (m, 1H).

M44: Colorless syrup. Yield 79%. ¹H NMR (500 MHz, CDCl₃) δ 8.14 (br, 1H), 7.61 (br, 1H), 7.35-7.19 (m, 4H), 7.11-7.09 (m, 2H), 7.07-6.91 (m, 6H), 4.94 (d, J=2.9 Hz, 1H), 4.77 (dd, J=48.0, 11.5 Hz, 2H), 4.60-4.50 (m, 3H), 4.47 (d, J=12.4 Hz, 1H), 4.45-4.34 (m, 2H), 4.18-4.15 (m, 1H), 3.74-3.71 (m, 1H), 3.57-3.54 (m, 1H).

M45: White solid. Yield 85%. ¹H NMR (500 MHz, CDCl₃) δ 9.06 (br, 1H), 7.56-7.52 (m, 1H), 7.36-7.20 (m, 6H), 7.16-6.97 (m, 5H), 4.98-4.96 (m, 2H), 4.73 (d, J=12.2 Hz, 1H), 4.60 (d, J=11.4 Hz, 1H), 4.56-4.48 (m, 2H), 4.43 (d, J=11.8 Hz, 1H), 4.37 (d, J=4.5 Hz, 1H), 4.33-4.31 (m, 1H), 4.04-4.02 (m, 1H), 3.98-3.96 (m, 1H), 3.66-3.63 (m, 1H).

M46: Colorless syrup. Yield 75%. ¹H NMR (500 MHz, CDCl₃) δ 8.12 (br, 1H), 7.56 (br, 1H), 7.48-7.45 (m, 1H), 7.36-7.19 (m, 6H), 7.14-6.94 (m, 5H), 4.94 (d, J=3.0 Hz, 1H), 4.88-4.79 (m, 2H), 4.64-4.51 (m, 5H), 4.36-4.33 (m, 1H), 4.20-4.17 (m, 1H), 3.76-3.73 (m, 1H), 3.58-3.55 (m, 1H).

M57: Colorless syrup. Yield 43%. ¹H NMR (500 MHz, CDCl₃) δ 7.38-7.26 (m, 15H), 4.65-4.61 (m, 3H), 4.60-4.57 (m, 2H), 4.54-4.47 (m, 2H), 4.31 (t, J=5.1 Hz, 1H), 4.24 (dd, J=8.2, 3.6 Hz, 1H), 4.05 (t, J=4.7 Hz, 1H), 3.59-3.50 (m, 2H).

M58: Colorless syrup. Yield 24.5%. ¹H NMR (500 MHz, CDCl₃) δ 7.41-7.26 (m, 14H), 7.22-7.20 (m, 1H), 4.84-4.66 (m, 3H), 4.65-4.29 (m, 6H), 4.19-4.12 (m, 1H), 4.03-3.90 (m, 1H), 3.56-3.44 (m, 2H).

M59: Colorless syrup. Yield 66%. ¹H NMR (500 MHz, CDCl₃) δ 7.37-7.31 (m, 2H), 7.29-7.22 (m, 4H), 7.06-6.96 (m, 6H), 4.75-4.73 (m, 1H), 4.62-4.32 (m, 7H), 4.12-4.10 (m, 1H), 3.92-3.91 (m, 1H), 3.74-3.56 (m, 2H).

TABLE 1
Structures of M26 and M26 Analogues of Class A.

| | |
|---|---|
| ID | Structure |

| | |
|---|---|
| M26 | 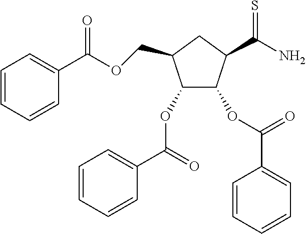 [SMILES: NC(=S)[C@@H]1C[C@H](COC(=O)c2ccccc2)[C@@H](OC(=O)c2ccccc2)[C@H]1OC(=O)c1ccccc1] |
| M33 | 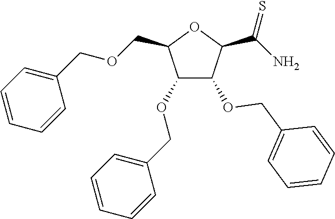 [SMILES: NC(=S)[C@@H]1O[C@H](COCc2ccccc2)[C@@H](OCc2ccccc2)[C@H]1OCc1ccccc1] |
| M34 | 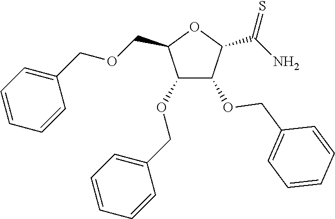 [SMILES: NC(=S)[C@H]1O[C@H](COCc2ccccc2)[C@@H](OCc2ccccc2)[C@H]1OCc1ccccc1] |
| M36 | 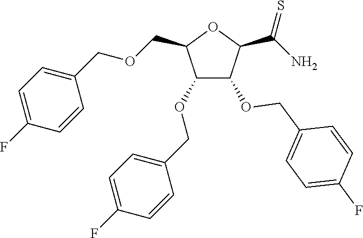 [SMILES: NC(=S)[C@@H]1O[C@H](COCc2ccc(F)cc2)[C@@H](OCc2ccc(F)cc2)[C@H]1OCc1ccc(F)cc1] |
| M39 | 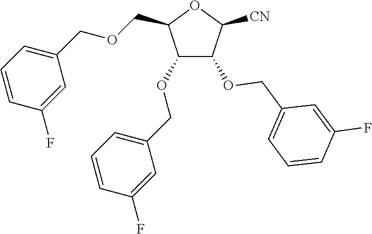 [SMILES: N#C[C@@H]1O[C@H](COCc2cccc(F)c2)[C@@H](OCc2cccc(F)c2)[C@H]1OCc1cccc(F)c1] |
| M40 | 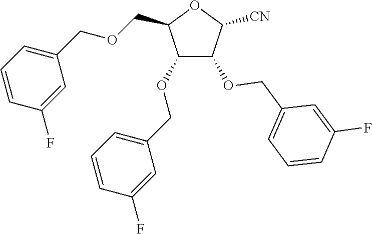 [SMILES: N#C[C@H]1O[C@H](COCc2cccc(F)c2)[C@@H](OCc2cccc(F)c2)[C@H]1OCc1cccc(F)c1] |
| M41 | 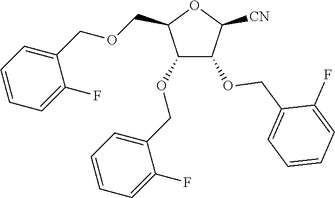 [SMILES: N#C[C@@H]1O[C@H](COCc2ccccc2F)[C@@H](OCc2ccccc2F)[C@H]1OCc1ccccc1F] |
| M42 | 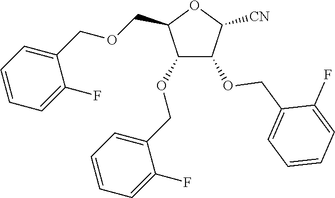 [SMILES: N#C[C@H]1O[C@H](COCc2ccccc2F)[C@@H](OCc2ccccc2F)[C@H]1OCc1ccccc1F] |
| M43 | 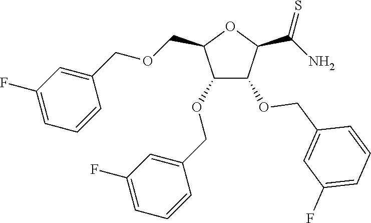 [SMILES: NC(=S)[C@@H]1O[C@H](COCc2cccc(F)c2)[C@@H](OCc2cccc(F)c2)[C@H]1OCc1cccc(F)c1] |
| M44 | 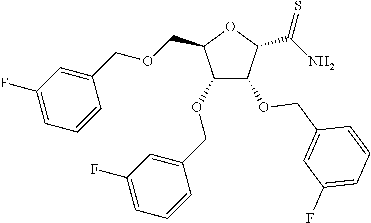 [SMILES: NC(=S)[C@H]1O[C@H](COCc2cccc(F)c2)[C@@H](OCc2cccc(F)c2)[C@H]1OCc1cccc(F)c1] |
| M45 | 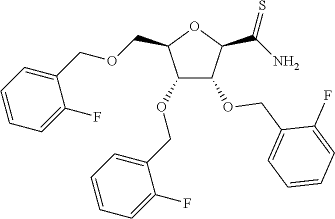 [SMILES: NC(=S)[C@@H]1O[C@H](COCc2ccccc2F)[C@@H](OCc2ccccc2F)[C@H]1OCc1ccccc1F] |
| M46 | 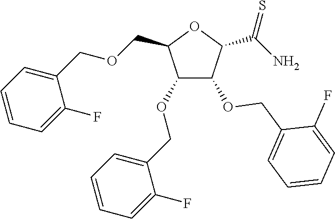 [SMILES: NC(=S)[C@H]1O[C@H](COCc2ccccc2F)[C@@H](OCc2ccccc2F)[C@H]1OCc1ccccc1F] |
| M57 | 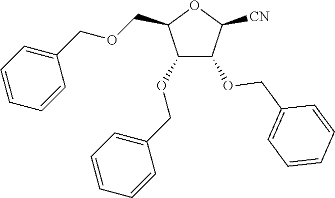 [SMILES: N#C[C@@H]1O[C@H](COCc2ccccc2)[C@@H](OCc2ccccc2)[C@H]1OCc1ccccc1] |
| M58 | 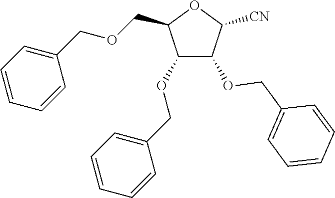 [SMILES: N#C[C@H]1O[C@H](COCc2ccccc2)[C@@H](OCc2ccccc2)[C@H]1OCc1ccccc1] |
| M59 | 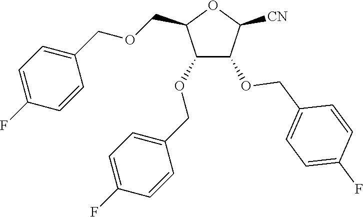 [SMILES: N#C[C@@H]1O[C@H](COCc2ccc(F)cc2)[C@@H](OCc2ccc(F)cc2)[C@H]1OCc1ccc(F)cc1] |
| M88 | 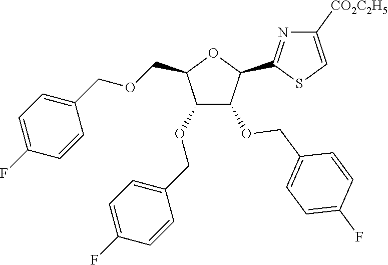 [SMILES: CCOC(=O)c1csc([C@@H]2O[C@H](COCc3ccc(F)cc3)[C@@H](OCc3ccc(F)cc3)[C@H]2OCc2ccc(F)cc2)n1] |

### Example 2—Preparation of M26 Analogues of Class B˜D

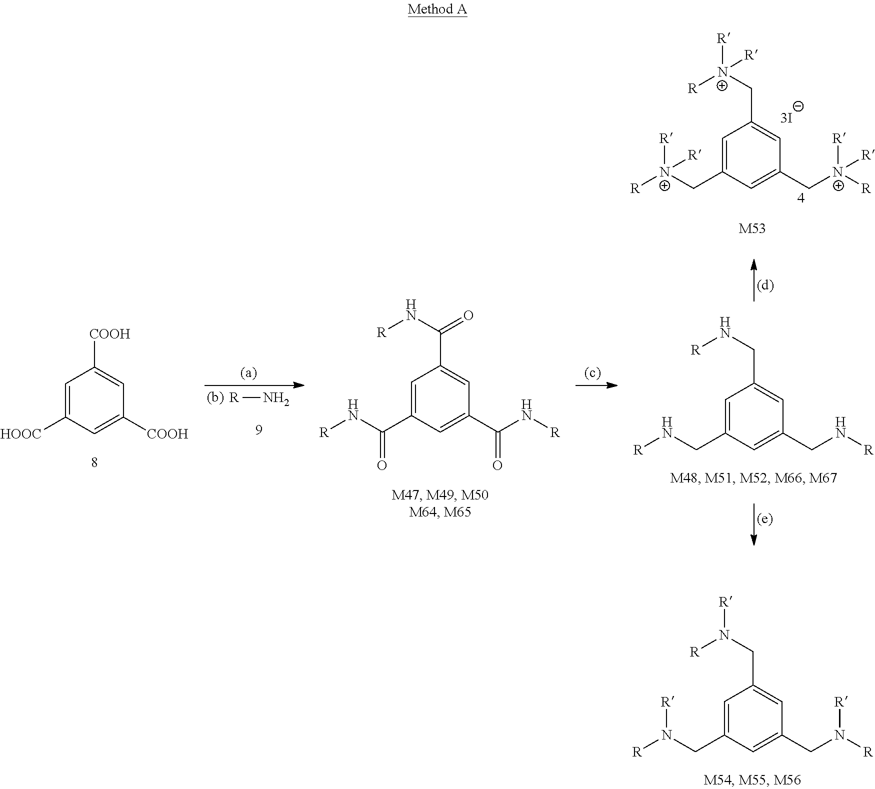

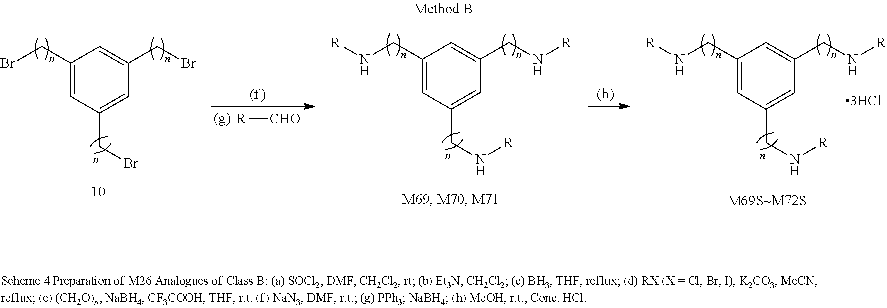

Compounds of M26 Analogues of Class B can be prepared by two typical methods as illustrated in Scheme 4. In Method A, amides M47, M49, M50, M64 and M65 were obtained by the condensation of benzenetricarboxylic acid 8 with amines 9. Subsequent reduction of these amides using borane, amines M48, M51, M52, M66 and M67 were obtained. By the further alkylation of these amines, tertiary amines M54˜M56 and quaternary ammonium salt M53 were obtained.

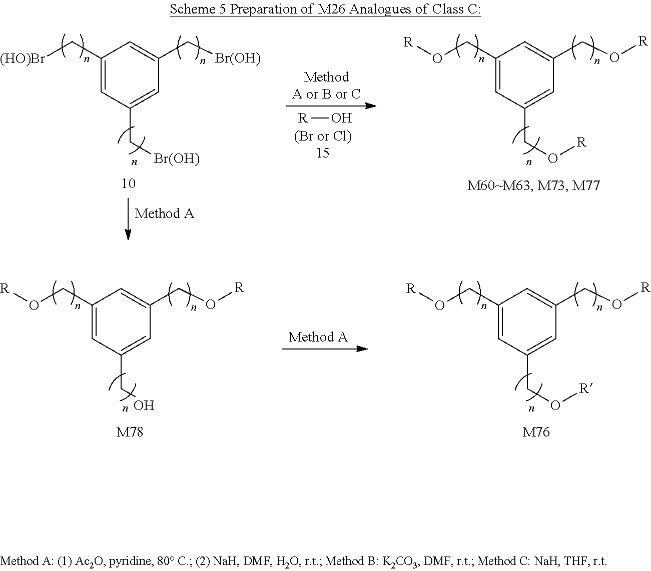

Compounds of M26 Analogues of Class C can be prepared by typical methods as illustrated in Scheme 5. Symmetric and unsymmetric ethers M60˜M63, M73, M76, M77 and M78 were obtained by using typical methods A, B and C.

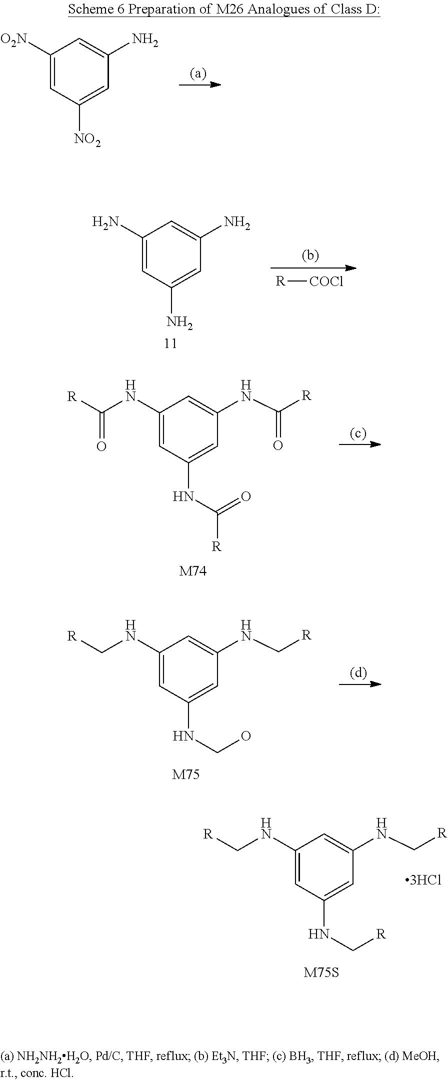

Compounds of M26 Analogues of Class D can be prepared by typical methods as illustrated in Scheme 6. Amide M74 were obtained by the reaction of 1,3,5-benzenetriamine 11 with acyl chloride. Subsequent reduction of these amides using borane, amine M75 was obtained.

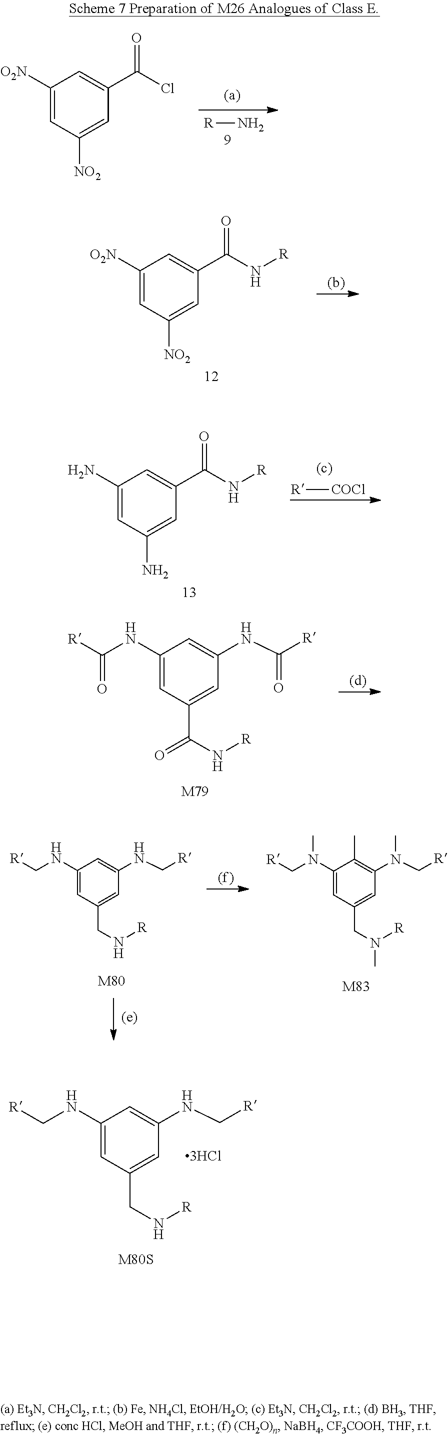

Compounds of M26 Analogues of Class E can be prepared by typical methods as illustrated in Scheme 7. Amid 12 were obtained by the reaction of acyl chloride with amine 9. Subsequent reduction of nitryl using Fe gave amine 13. Further acylation of amine 18 gave amide M79. Reduction of M79 by BH₃ gave M80. Methylation of M80 by (CH₂O)ₙ gave M83. And the salt form of M80S was obtained by the treatment of M80 with conc. HCl in CH₃OH.

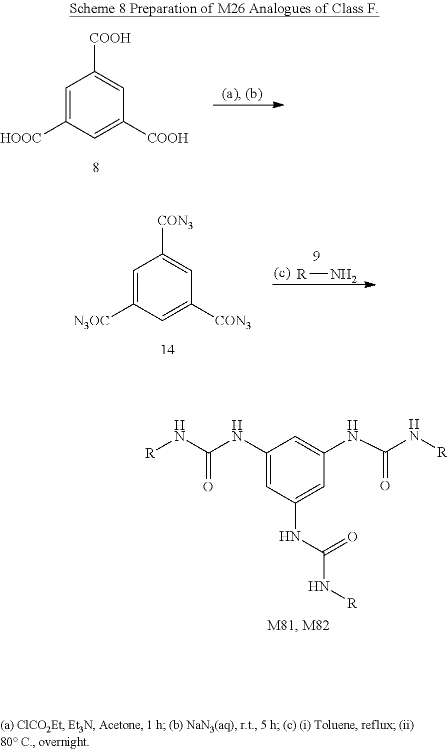

Compounds of M26 Analogues of Class F can be prepared by typical methods as illustrated in Scheme 8. M81 and M82 were obtained by the typical procedure for the synthesis of aryl urea.

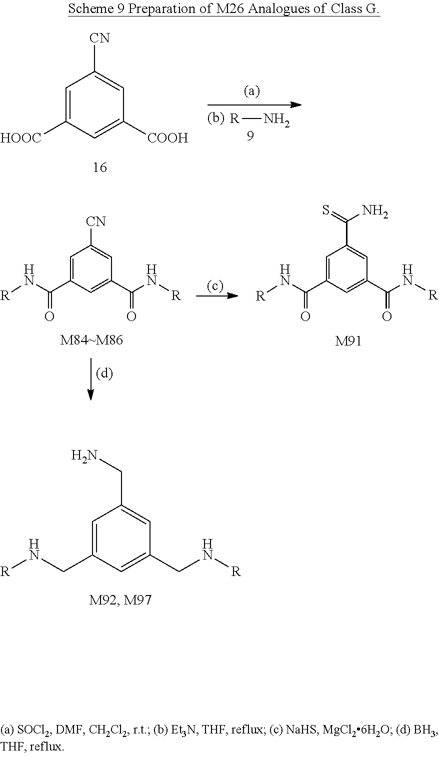

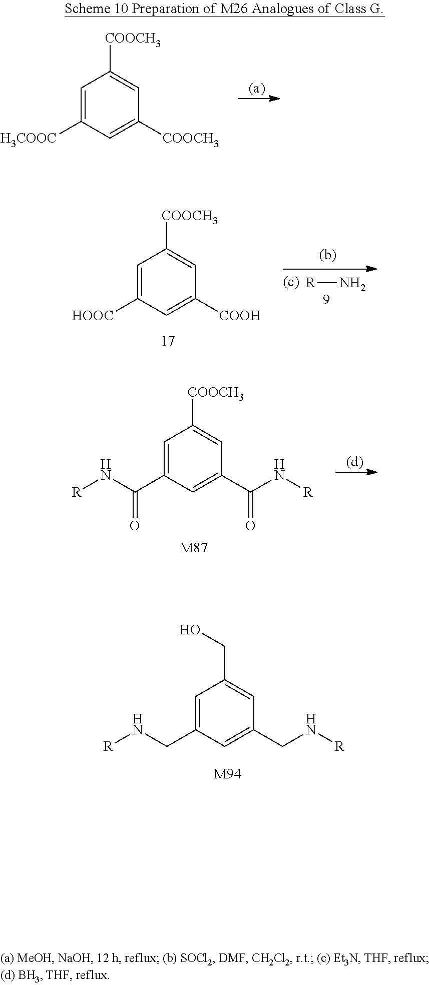

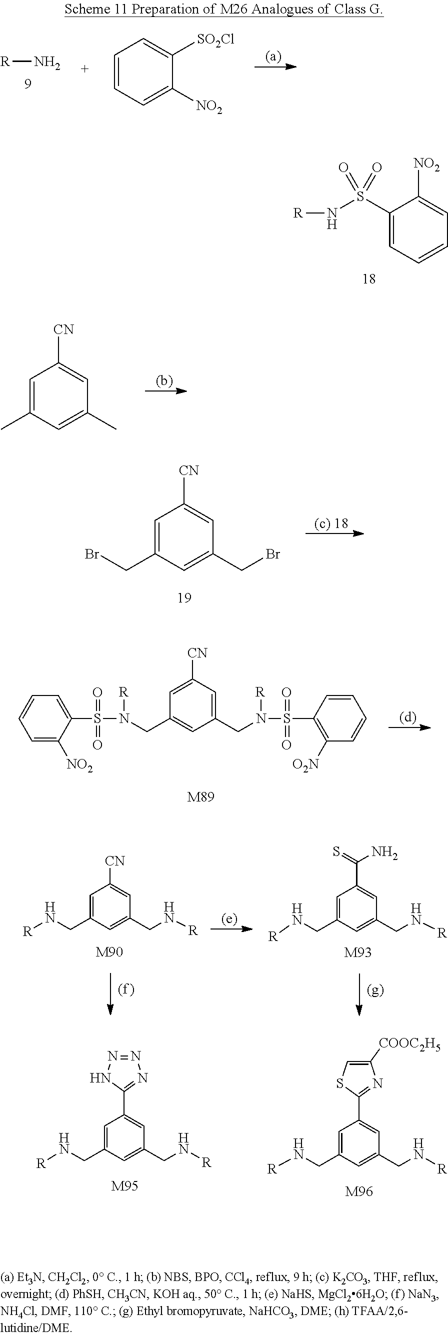

Compounds of M26 Analogues of Class G can be prepared by typical methods as illustrated in Scheme 9˜11. M84˜M86 and M87 were obtained by the typical procedure for the preparation of amid. Subsequent reduction of M84˜M86 and M87 by using BH₃ gave M92, M97 and M94 respectively. Treatment of M86 and M90 with NaHS and MgCl₂.6H₂O in DMF at r.t., M91 and M93 were obtained. Further treatment of M90 with NaN₃, NH₄Cl in DMF at reflux gave M95. M96 was obtained by the treatment of M93 with ethylbromopyruvate and NaHCO₃ in dry DME, and then by addition of a mixture of trifluoroacetic anhydride and 2,6-lutidine in dry 1,2-dimethoxyethane.

General Procedures for the Preparation of M26 Analogues of Class B:

Method A—General procedure for the preparation of M47, M49, M50, M64 and M65:

A mixture of 1,3,5-benzenetricarboxylic acid 8 (0.21 g, 1 mmol), SOCl₂ (2 mL, 28 mmol) and two drops of DMF was heated under reflux for 3 hours. After cooling to room temperature, the excess SOCl₂ was removed in vacuo to give 1,3,5-benzenetricarboxylic chloride, which was used without further purification. To a mixture of amine 9 (3.3 mmol), and Et₃N (1.4 mL, 10 mmol) in 10 mL CH₂Cl₂ at 0° C., 1,3,5-benzenetricarboxylic chloride in CH₂Cl₂ was added slowly. The mixture was stirred at r.t. for 4 hours. The reaction mixture was washed with water, brine. The combined organic layers were dried over anhydrous Na₂SO₄ and concentrated in vacuo. The crude residue was recrystalized from EtOH to afford pure product amide M47, M49, M50, M64 and M65.

General Procedure for the Preparation of M48, M51, M52, M66 and M67:

To a solution of amide M47 (0.5 mmol) in THF (10 mL), borane (8 mL of 1M solution in THF, 8 mmol) was added. The reaction mixture was heated at 70° C. overnight. After cooling to 0° C., 5M HCl (2 mL) and MeOH (3 mL) were added. The resulting mixture was stirred at r.t. for 4 hours, adjusted the pH to 12 with 6M NaOH, extracted with EtOAc. The combined organic layers were washed with brine, dried over anhydrous Na₂SO₄, concentrated in vacuo and the crude residue was purified by flash chromatography to give M48.

By using the same procedure, M51, M52, M66 and M67 were obtained from amides M49, M50, M64 and M65.

General Procedure for the Preparation of M54˜M56:

To a mixture of M51 (0.1 mmol), paraformaldehyde (60 mg, 2 mmol), and NaBH₄ (19 mg, 0.5 mmol) in 10 mL THF at r.t. under nitrogen, trifluoroacetic acid (2 mL) was added dropwise over 1 hour. The resulting mixture was stirred at r.t. for 24 hours. Then the mixture was concentrated in vacuo, diluted with EtOAc, the organic layer was washed with H₂O, NaHCO₃, brine, and dried over Na₂SO₄, filtered and the solvent was evaporated. The crude residue was purified by flash chromatography to give M56.

By using the same procedure, M54, M55 were obtained from amide M48, M52.

General Procedure for the Preparation of M53:

A mixture of the M48 (24.5 mg, 0.05 mmol), MeI (142 mg, 1 mmol) and K₂CO₃ (138 mg, 1 mmol) in 5 mL of acetonitrile was refluxed overnight. The mixture was cooled at r.t. and filtered. Then the organic layer was further cooled to −20° C. for 5 hours. Light yellow precipitation was formed, which was collected by filtration to give M53.

Method B—General Procedure for the Preparation of M69˜M71:

1,3,5-tris(bromomethyl)benzene (1.07 g, 3.0 mmol) and NaN₃ (1.17 g, 18 mmol) were dissolved in 12 mL DMF. The reaction was stirred for 12 hours at 80° C., then treated with H₂O and extracted with ethyl acetate, washed with brine. The organic layer was dried over anhydrous sodium sulfate, filtered and the solvent was evaporated. The crude residue was purified by column chromatography to give triazide as a colourless syrup (0.91 g).

A solution of triazide (1.0 mmol) and arylaldehyde (3.3 mmol) in anhydrous THF (5 mL) in the presence of triphenylphosphine (3.3 mmol) was stirred at room temperature. After 24 hours, the reaction mixture was diluted with MeOH (10 mL) and subsequently added NaBH₄ (3.3 mmol). Then the reaction was stirred overnight at room temperature. After evaporation, the residue was partitioned in CH₂Cl₂ and saturated Na₂CO₃ aqueous solution and extracted with CH₂Cl₂. The organic layer was dried over anhydrous sodium sulfate, filtered and the solvent was evaporated. The crude residue was purified by column chromatography to give M69, M70, M71.

General Procedure for the Preparation of M69S˜M72S.

To a solution of M69 in MeOH, conc. HCl was added dropwise at r.t. The reaction mixture was stirred at r.t. for 1 hour. Then THF was added and white precipitate was formed, which was collected by filtration to give M69S. By using the same procedure, M70S˜M72S were obtained.

General Procedures for the Preparation of M26 Analogues of Class C:

Method A: To a solution of 1,3,5-trihydroxybenzene (2.5 g) in pyridine (12 mL) was added acetic anhydride (11.2 mL) and after refluxed for 12 hours, the solution was poured into iced water which led to formation of a white precipitate. After stirring for 2 hours, the solid was collected by filtration, and recrystallized from ethanol to give benzene-1,3,5-triacetate (3 g).

To a mixture of benzene-1,3,5-triacetate (252 mg, 1 mmol), benzyl chloride (443 mg, 3.5 mmol), 60% NaH in mineral oil (280 mg, 7 mmol) and DMF (5 mL), H₂O (54 mg, 3 mmol) was added at 0° C. dropwise. After stirring for 2 hours at room temperature, the reaction mixture was diluted with ethyl acetate and washed with water and brine. The organic layer was dried over Na₂SO₄ and concentrated. The crude residue was purified by column chromatography to give M61, M62, M73 and M78.

To a mixture of M78 (1 mmol), chloride 15 (3.5 mmol), 60% NaH in mineral oil (280 mg, 7 mmol) and DMF (5 mL), H₂O (54 mg, 3 mmol) was added at 0° C. dropwise. After stirring for 2 hours at room temperature, the reaction mixture was diluted with ethyl acetate and washed with water and brine. The organic layer was dried over Na₂SO₄ and concentrated. The crude residue was purified by column chromatography to give M76.

Method B: A mixture of 1,3,5-trihydroxybenzene (63 mg, 0.5 mmol), 4-picolyl chloride hydrochloride (443 mg, 1.75 mmol) and K₂CO₃ (691 mg, 5 mmol) was stirred overnight. After the evaporation of DMF, water was added and white precipitate was formed, which was collected by filtration. Recrystallized from ethanol, M60 was obtained as pale yellow powder in 20.3% yield.

Method C: To a solution of 4-fluorobenzyl alcohol (315 mg, 2.5 mmol) and 1,3,5-tris(bromomethyl)benzene (179 mg, 0.5 mmol) in THF (80 mL), NaH (72 mg, 60% dispersion in mineral oil, 3 mmol) was added. The mixture was stirred at room temperature for 24 hours. The reaction mixture was poured into H₂O and filtered. The residue was washed with H₂O, dried in vacuo, and subjected to column chromatography to give 49 mg (yield: 20.0%) M63 as yellow syrup.

General Procedure for the Preparation of M26 Analogues of Class D:

To a mixture of amine 11 (200 mg, 1.6 mmol) and Et₃N (0.5 mL, 3.6 mmol) in THF was added acyl chloride (0.75 mL, 6.4 mmol) dropwise with an ice-water bath. After 2 hours, water was added to the reaction mixture. 220 mg (31.6% yield) amide M74 was obtained by filtration.

To M74 (200 mg, 0.46 mmol) in dry THF, a solution of borane (8 mL of 1M solution in THF, 8 mmol) was added. The reaction mixture was heated at 70° C. overnight. After cooling to 0° C., 5M HCl (2 mL) and MeOH (3 mL) were added. The resulting mixture was stirred at r.t. for 4 hours, adjusted the pH to 12 with 6M NaOH, extracted with EtOAc. The combined organic layers were washed with brine, dried over anhydrous Na₂SO₄, concentrated in vacuo. The crude residue was purified by flash chromatography to give M75 (122 mg, 67.5% yield).

M75S were obtained by using the same procedure for the preparation of M69S.

General Procedure for the Preparation of M26 Analogues of Class E:

M79 were obtained by using the same procedure for the preparation of M47.

M80 were obtained by using the same procedure for the preparation of M48

M80S were obtained by using the same procedure for the preparation of M69S.

Preparation of M83: To a mixture of M80 (50 mg, 0.123 mmol), paraformaldehyde (74 mg, 2.45 mmol), and NaBH₄ (47 mg, 1.23 mmol) in 3 mL THF at r.t. under nitrogen, trifluoroacetic acid (1 mL) was added dropwise. The resulting mixture was stirred at r.t. for 24 hours. Then the mixture was concentrated in vacuo, adjusted the pH>11 with NaOH solution, diluted with EtOAc, the organic layer was washed with H₂O, brine, and dried over Na₂SO₄, filtered and the solvent was evaporated. The crude residue was purified by flash chromatography to give M83.

General Procedure for the Preparation of M26 Analogues of Class F:

To a solution of 8 (1 mmol) in dry acetone (10 mL), triethylamine (1.1 mmol) and ethyl chlorocarbamate (1.1 mmol) were added dropwise at 0° C. After stirring at 0° C. for 1 hour, sodium azide (1.1 mmol, 0.215 g) dissolved in 5 mL water was added dropwise. Stirring was continued at 0° C. for 5 hours. Ice water was added. The mixture was extracted by dichloroform (3×20 mL). The combined organic layers were washed with brine and dried over Na₂SO₄. The organic phase was concentrated under reduced pressure. Colorless oil 17 was obtained and used in the following reaction without further purification.

A solution of aryl azide 17 (0.5 mmol) in toluene (10 mL) was heated at 110° C. for 3 hours. After cooling to rt, amine 9 was added. The reaction moxturewas heated at 90° C. overnight. The reaction was cooled to room temperature and the precipitate was collected by filtration and washed with toluene to give M81 and M82.

General Procedure for the Preparation of M26 Analogues of Class G:

M84˜M86 and M88 were obtained by using the same procedure for the preparation of M47.

Preparation of M91: A mixture of M86 (1 mmol), NaHS (2 mmol) and MgCl₂.6H₂O (1 mmol) in DMSO was stirred at r.t. for 6 hours. Then water was added and extracted with CH₂Cl₂. The organic layer was washed with H₂O, brine, and dried over Na₂SO₄, filtered and the solvent was evaporated. The crude residue was purified by flash chromatography to give M91.

M92, M97 and M94 were obtained by using the same procedure for the preparation of M48.

Preparation of M89: A mixture of 18 (0.5 mmol), 19 (0.55 mmol) and 0.21 g (1.5 mmol) of potassium carbonate in 10 mL of anhydrous THF was heated under reflux for 6 hours. The reaction mixture is allowed to cool to room temperature, diluted with 250 mL of water, and extracted with dichloromethane (3×15 mL). The combined organic extracts were washed with brine (10 mL), dried over Na₂SO₄, filtered, and concentrated in vacuum. The crude residue is purified by column chromatography on silica gel to give M89.

Preparation of M90: To a solution of 0.11 mL (1 mmol) of thiophenol in 10 mL of acetonitrile, 0.1 mL 10.9 M aqueouspostassium hydroxide solution (1 mmol) is added dropwise at 0° C. Then the reaction mixture is allowed to warm to room temperature and 0.24 g (0.42 mmol) of M89 in 5 mL of acetonitrile was added dropwise. The reaction mixture is heated in a 50° C. oil bath for 40 minutes. After cooling to room temperature, 10 mL water was added, and extracted with dichloromethane (3×15 mL). The combined organic extracts were washed with brine, dried over Na₂SO₄, filtered, and concentrated under reduced pressure. The residue is purified by column chromatography to give M90.

M93 were obtained by using the same procedure for the preparation of M91.

Preparation of M95: A stirred mixture of the M90 (1 mmol), sodium azide (2.2 mmol) in 10 mL DMF was heated overnight at 110° C. The reaction mixture is allowed to cool to room temperature, water was added and adjust the PH˜3. Extracted with dichloromethane (3×15 mL). The combined organic extracts were washed with brine (10 mL), dried over Na₂SO₄, filtered, and concentrated in vacuum. The crude residue is purified by column chromatography on silica gel to give M89.

M96 were obtained by using the same procedure for the preparation of M87.

Characterization of M26 Analogues of Class B˜D:

M47: White solid, 75.3% yield. ¹H NMR (500 MHz, DMSO-d₆) δ 9.27 (t, J=5.9 Hz, 3H), 8.49 (s, 3H), 7.41-7.34 (m, 6H), 7.19-7.11 (m, 6H), 4.47 (d, J=5.9 Hz, 6H).

M48: Colorless oil, 64.7% yield. ¹H NMR (500 MHz, Acetone-d₆) δ 7.46-7.39 (m, 6H), 7.28 (s, 3H), 7.11-6.97 (m, 6H), 3.78 (d, J=4.9 Hz, 12H), 2.64 (brs, 3H). HRMS (ESI) m/z Found: 490.2457 [M+H]⁺, Calcd: 490.2465.

M49: White solid, 56.4% yield. ¹H NMR (500 MHz, DMSO-d₆) δ 9.26 (t, J=6.0 Hz, 3H), 8.50 (s, 3H), 7.37-7.29 (m, 9H), 7.28-7.20 (m, 3H), 4.50 (d, J=5.9 Hz, 6H).

M50: Light yellow solid, 80.3% yield. ¹H NMR (500 MHz, DMSO-d₆) δ 9.16 (t, J=5.9 Hz, 3H), 8.45 (s, 3H), 7.32-7.23 (m, 6H), 6.92-6.85 (m, 6H), 4.42 (d, J=5.9 Hz, 6H), 3.72 (s, 9H).

M51: Colorless oil, 50.3% yield. ¹H NMR (500 MHz, Acetone-d₆) δ 7.43-7.38 (m, 5H), 7.37-7.19 (m, 10H), 3.79 (d, J=9.1 Hz, 12H), 2.80 (brs, 3H). MS (ESI) m/z Found: 436.32 [M+H]⁺, Calcd: 436.28.

M51S: White solid, 90.1% yield. ¹H NMR (500 MHz, D₂O) δ 7.44 (s, 3H), 7.40-7.31 (m, 15H), 4.22 (s, 6H), 4.18 (s, 6H).

M52: Colorless oil, 59.9% yield. ¹H NMR (500 MHz, Acetone-d₆) δ 7.34-7.22 (m, 9H), 6.91-6.83 (m, 6H), 3.78 (s, 9H), 3.76 (s, 6H), 3.73 (s, 6H), 2.86 (brs, 2H).

M53: Light yellow solid (15 mg, 42.7% yield). ¹H NMR (500 MHz, D₂O) δ 7.96 (s, 3H), 7.68-7.60 (m, 6H), 7.34-7.26 (m, 6H), 4.72 (s, 6H), 4.68 (s, 6H), 3.05 (s, 18H).

M54: Colorless oil, 52.9% yield. ¹H NMR (500 MHz, Acetone-d₆) δ 7.46-7.36 (m, 6H), 7.30 (s, 3H), 7.10-7.02 (m, 6H), 3.54 (s, 6H), 3.49 (s, 6H), 2.14 (s, 9H).

M55: Colorless oil, 51.4% yield. ¹H NMR (500 MHz, Acetone-d₆) δ 7.37-7.26 (m, 9H), 6.88 (d, J=8.5 Hz, 6H), 3.78 (s, 9H), 3.55 (s, 6H), 3.47 (s, 6H), 2.15 (s, 9H). MS (ESI) m/z Found: 568.41 [M+H]⁺, Calcd: 568.35.

M56: Colorless oil, 43.5% yield. ¹H NMR (500 MHz, Acetone-d₆) δ 7.58-7.19 (m, 18H), 3.62 (s, 12H), 2.20 (s, 9H).

M60: White solid, 41.4% yield. ¹H NMR (500 MHz, Acetone-d₆) δ 8.60 (d, J=5.9 Hz, 6H), 7.44 (d, J=5.4 Hz, 6H), 6.40 (s, 3H), 5.21 (s, 6H).

M61: White solid, 80.1% yield. ¹H NMR (500 MHz, Acetone-d₆) δ 7.47 (d, J=7.3 Hz, 6H), 7.44-7.30 (m, 9H), 6.32 (s, 3H), 5.10 (s, 6H).

M62: White solid, 38.8% yield. ¹H NMR (500 MHz, CDCl₃) δ 7.42-7.35 (m, 6H), 7.12-7.04 (m, 6H), 6.24 (s, 3H), 4.97 (s, 6H).

M63: Colorless oil, 30.2% yield. ¹H NMR (500 MHz, CDCl₃) δ 7.37-7.30 (m, 6H), 7.29 (s, 3H), 7.06-6.99 (m, 6H), 4.55 (s, 6H), 4.52 (s, 6H).

M64: White solid, 66.3% yield. ¹H NMR (500 MHz, DMSO-d₆) δ 9.32 (t, J=5.9 Hz, 3H), 8.52 (s, 3H), 7.41-7.33 (m, 3H), 7.22-7.11 (m, 6H), 7.11-7.03 (m, 3H), 4.51 (d, J=5.9 Hz, 6H).

M65: White solid, 60.4% yield. ¹H NMR (500 MHz, DMSO-d₆) δ 9.27 (t, J=5.7 Hz, 3H), 8.52 (s, 3H), 7.46-7.29 (m, 6H), 7.23-7.13 (m, 6H), 4.54 (d, J=5.6 Hz, 6H).

M66: Colorless oil, 50.3% yield. ¹H NMR (500 MHz, CDCl₃) δ 7.31-7.25 (m, 3H), 7.21 (s, 3H), 7.14-7.05 (m, 6H), 6.98-6.90 (m, 3H), 3.82 (s, 6H), 3.79 (s, 6H).

M67: Colorless oil, 55.9% yield. ¹H NMR (500 MHz, CDCl₃) δ 7.41-7.33 (m, 3H), 7.29-7.23 (m, 3H), 7.22 (s, 3H), 7.15-7.00 (m, 6H), 3.88 (s, 6H), 3.81 (s, 6H).

M68: Yellow oil, 30.2% yield. ¹H NMR (500 MHz, Acetone-d₆) δ 8.50-8.45 (m, 6H), 7.38-7.33 (m, 6H), 7.27 (s, 3H), 3.81 (s, 6H), 3.78 (s, 6H).

M69: Yellow oil, 48.8% yield. ¹H NMR (500 MHz, Acetone-d₆) δ 8.58 (d, J=1.7 Hz, 3H), 8.45 (dd, J=4.7, 1.5 Hz, 3H), 7.81-7.75 (m, 3H), 7.34-7.20 (m, 6H), 3.82 (s, 6H), 3.79 (s, 6H).

M69S: White solid, 40.8% yield. ¹H NMR (500 MHz, D₂O) δ 8.51-8.43 (m, 6H), 7.86 (d, J=8.0 Hz, 3H), 7.49 (s, 3H), 7.46-7.38 (m, 3H), 4.25 (s, 12H).

M70: Colorless syrup, 89.8% yield. ¹H NMR (500 MHz, CDCl₃) δ 7.39-7.34 (m, 3H), 7.18 (s, 3H), 6.34-6.29 (m, 3H), 6.21-6.16 (m, 3H), 3.80 (s, 6H), 3.77 (s, 6H), 1.81 (brs, 3H).

M70S: White solid, 78.1% yield. ¹H NMR (500 MHz, D₂O) δ 7.48 (s, 3H), 7.46 (s, 3H), 6.51 (d, J=3.1 Hz, 3H), 6.39 (s, 3H), 4.23 (s, 6H), 4.21 (s, 6H).

M71: Colorless syrup, 91.5% yield. ¹H NMR (500 MHz, CDCl₃) δ 7.25-7.14 (m, 6H), 6.98-6.91 (m, 6H), 4.00 (s, 6H), 3.83 (s, 6H), 1.73 (brs, 3H).

M71S: White solid, 61.4% yield. ¹H NMR (500 MHz, D₂O) δ 7.50-7.43 (m, 6H), 7.20-7.14 (m, 3H), 7.04-6.99 (m, 3H), 4.42 (s, 6H), 4.23 (s, 6H).

M72S: White solid, 38.3% yield. ¹H NMR (500 MHz, D₂O) δ 8.64-8.56 (m, 3H), 8.20-8.06 (m, 3H), 7.74-7.68 (m, 3H), 7.67-7.60 (m, 6H), 4.47 (s, 6H), 4.38 (s, 6H).

M73: Light yellow solid, 25.6% yield. ¹H NMR (500 MHz, Acetone-d₆) δ 7.49 (dd, J=5.1, 1.2 Hz, 3H), 7.23-7.18 (m, 3H), 7.04 (dd, J=5.1, 3.5 Hz, 3H), 6.34 (s, 3H), 5.29 (s, 6H).

M74: White solid, 41.3% yield. ¹H NMR (500 MHz, DMSO-d₆) δ 10.38 (s, 3H), 8.05 (s, 3H), 8.02-7.97 (m, 6H), 7.63-7.50 (m, 9H).

M75: Black syrup, 70.9% yield. ¹H NMR (500 MHz, Acetone-d₆) δ 7.40-7.15 (m, 15H), 5.48-5.38 (s, 3H), 4.85 (brs, 3H), 4.19 (s, 6H).

M75S: White solid, 33.9% yield. ¹H NMR (500 MHz, DMSO-d₆) δ 7.33 (m, 15H), 6.12 (s, 1H), 6.00 (s, 2H), 4.28 (s, 6H). MS (ESI) m/z Found: 394.21 [M+H]⁺, Calcd: 394.23.

M76: White solid, 40.5% yield. ¹H NMR (500 MHz, Acetone-d₆) δ 7.54-7.43 (m, 5H), 7.42-7.28 (m, 4H), 7.20-7.11 (m, 4H), 6.33-6.27 (m, 3H), 5.08 (s, 2H), 5.07 (s, 4H). HRMS (ESI) m/z Found: 433.1608 [M+H]⁺, Calcd: 433.1610.

M77: White solid, 37.6% yield. ¹H NMR (500 MHz, Acetone-d₆) δ 7.43-7.42 (m, 3H), 7.30 (s, 3H), 7.07-7.06 (m, 3H), 7.00-6.99 (m, 3H), 4.74-4.73 (m, 6H), 4.59-4.56 (m, 6H).

M78: White solid, 40.8% yield. ¹H NMR (500 MHz, CDCl₃) δ 7.40-7.34 (m, 4H), 7.07 (t, J=8.7 Hz, 4H), 6.46 (t, J=2.2 Hz, 1H), 6.36 (d, J=2.2 Hz, 2H), 4.96 (s, 4H), 2.28 (s, 3H).

M79: White solid, 77.3% yield. ¹H NMR (500 MHz, DMSO-d₆) δ 10.45 (s, 2H), 9.01 (s, 1H), 8.47 (s, 1H), 8.10-7.92 (m, 6H), 7.76-7.48 (m, 6H), 7.43-7.21 (m, 4H), 4.48 (s, 2H).

M80: Light black oil, 47.1% yield. ¹H NMR (500 MHz, CDCl₃) δ 7.38-7.22 (m, 15H), 6.06 (d, J=1.9 Hz, 2H), 5.83 (t, J=1.9 Hz, 1H), 4.28 (s, 4H), 3.77 (s, 2H), 3.64 (s, 2H). HRMS (ESI) m/z Found: 408.24546 [M+H]⁺, Calcd: 408.24342.

M80S: White solid, 67.9% yield. ¹H NMR (500 MHz, D₂O) δ 7.55-7.45 (m, 3H), 7.41-7.22 (m, 12H), 6.64-6.58 (m, 1H), 4.46 (s, 4H), 4.07 (s, 2H), 3.78 (s, 2H).

M81: White solid, 69.8% yield. ¹H NMR (500 MHz, DMSO-d₆) δ 8.73 (s, 3H), 8.51 (s, 3H), 7.49-7.42 (m, 6H), 7.32 (s, 3H), 7.31-7.24 (m, 6H), 7.00-6.93 (m, 3H).

M82: White solid, 63.4% yield. ¹H NMR (500 MHz, DMSO-d₆) δ 8.47 (s, 3H), 7.42-7.02 (m, 18H), 6.44 (t, J=6.0 Hz, 3H), 4.27 (d, J=5.9 Hz, 6H).

M83: Colourless oil, 40.2% yield. ¹H NMR (500 MHz, CDCl₃) δ 7.56-7.10 (m, 15H), 6.66 (s, 1H), 6.49 (d, J=2.6 Hz, 1H), 4.50 (s, 2H), 3.99 (s, 2H), 3.50 (d, J=14.8 Hz, 4H), 2.98 (s, 3H), 2.53 (s, 3H), 2.32 (s, 3H), 2.15 (s, 3H). HRMS (ESI) m/z Found: 464.30727 [M+H]⁺, Calcd: 464.30602.

M84: White solid, 87.6% yield. ¹H NMR (400 MHz, Acetone-d₆) δ 8.28 (s, 1H), 8.07-8.06 (m, 2H), 7.33-7.24 (m, 2H), 7.01-7.00 (m, 2H), 6.98-6.89 (m, 4H), 6.71 (s, 2H), 3.75-3.70 (m, 4H), 2.95 (t, J=7.0 Hz, 4H).

M85: White solid, 91.3% yield. ¹H NMR (400 MHz, Acetone-d₆) δ 8.57 (s, 1H), 8.32-8.31 (m, 2H), 8.19 (br, 2H), 7.35-7.27 (m, 8H), 7.24-7.21 (m, 2H), 3.71-3.66 (m, 4H), 2.97 (t, J=7.4 Hz, 4H).

M86: White solid, 85.4% yield. ¹H NMR (400 MHz, Acetone-d₆) δ 8.71 (s, 1H), 8.57 (br, 2H), 8.44 (s, 2H), 7.41 (d, J=7.5 Hz, 4H), 7.34 (t, J=7.5 Hz, 4H), 7.29-7.25 (m, 2H), 4.65 (d, J=6.0 Hz, 4H).

M87: White solid. Yield: 84.3%. ¹H NMR (400 MHz, DMSO-d₆) δ 9.40 (t, J=5.9 Hz, 2H), 8.72-8.58 (m, 3H), 7.38-7.32 (m, 8H), 7.29-7.22 (m, 2H), 4.52 (d, J=5.9 Hz, 4H), 3.93 (s, 3H).

M89: Colorless syrup, 86.7% yield. ¹H NMR (400 MHz, CDCl₃) δ 8.00-7.98 (m, 2H), 7.78-7.69 (m, 4H), 7.68-7.61 (m, 2H), 7.25-7.20 (m, 6H), 7.18-7.13 (m, 3H), 7.06-7.02 (m, 4H), 4.41 (s, 4H), 4.37 (s, 4H).

M90: Colorless syrup, 92.5% yield. ¹H NMR (400 MHz, CDCl₃) δ 7.60 (s, 1H), 7.58 (br, 2H), 7.40-7.33 (m, 8H), 7.33-7.27 (m, 4H), 3.84 (s, 4H), 3.83 (s, 4H).

M91: Yellow solid. Yield: 84.3%. ¹H NMR (400 MHz, DMSO-d₆) δ ¹H NMR (400 MHz, DMSO) δ 10.08 (br, 1H), 9.68 (br, 1H), 9.23 (t, J=5.9 Hz, 2H), 8.52-8.41 (m, 3H), 7.40-7.31 (m, 8H), 7.30-7.21 (m, 2H), 4.51 (d, J=5.9 Hz, 4H).

M92: Colorless syrup. Yield: 45.7%. ¹H NMR (400 MHz, CDCl₃) δ 7.41-7.32 (m, 10H), 7.24 (m, 3H), 5.32 (s, 2H), 3.90-3.78 (m, 12H).

M93: Yellow syrup. Yield: 45.7%. ¹H NMR (400 MHz, CDCl₃) δ 7.78 (br, 2H), 7.51 (s, 1H), 7.37-7.32 (m, 9H), 7.32-7.25 (m, 3H), 3.86-3.85 (m, 8H), 1.80 (br, 2H).

M94: Colorless syrup. Yield 29.4%. ¹H NMR (400 MHz, CDCl₃) δ 8.34-8.30 (m, 3H), 7.50-6.99 (m, 10H), 5.39 (s, 2H), 4.36-4.32 (m, 8H).

M95: Colorless syrup. Yield: 45.7%. ¹H NMR (400 MHz, CDCl₃) δ 8.61-8.46 (m, 2H), 8.08-8.05 (m, 1H), 7.50-6.99 (m, 11H), 5.44 (s, 2H), 4.50-4.45 (m, 8H).

M96: Colorless syrup. Yield: 37.1%. ¹H NMR (400 MHz, CDCl₃) δ 8.08 (s, 1H), 7.88-7.85 (m, 3H), 7.53-7.33 (m, 10H), 4.12-3.78 (m, 8H), 3.15 (q, J=7.3 Hz, 2H), 1.46 (t, J=7.3 Hz, 3H).

M97: Colorless syrup. Yield: 33.7%. ¹H NMR (400 MHz, CDCl₃) δ 7.41-7.33 (m, 8H), 7.31-7.26 (m, 2H), 7.22 (s, 1H), 5.32 (s, 2H), 3.94-3.79 (m, 14H), 2.13 (br, 2H).

TABLE 2
Structures of M26 Analogues of Class B~D.

| ID | Structure |
|---|---|
| M47 | 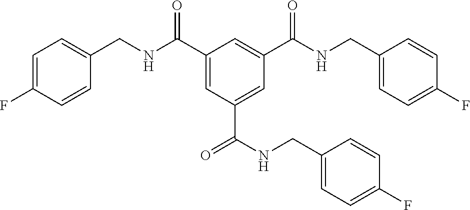 [SMILES: O=C(NCc1ccc(F)cc1)c1cc(C(=O)NCc2ccc(F)cc2)cc(C(=O)NCc2ccc(F)cc2)c1] |
| M48 | 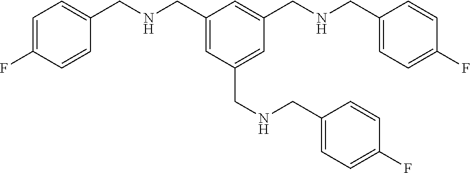 [SMILES: Fc1ccc(CNCc2cc(CNCc3ccc(F)cc3)cc(CNCc3ccc(F)cc3)c2)cc1] |
| M49 | 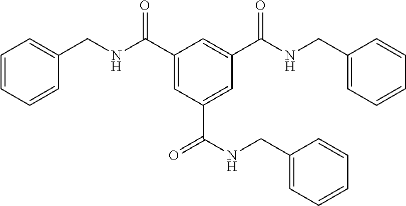 [SMILES: O=C(NCc1ccccc1)c1cc(C(=O)NCc2ccccc2)cc(C(=O)NCc2ccccc2)c1] |
| M50 | 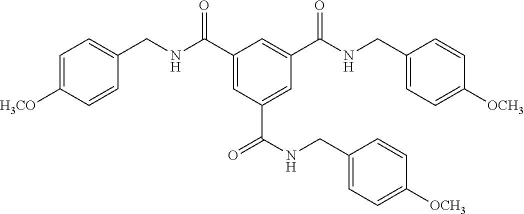 [SMILES: COc1ccc(CNC(=O)c2cc(C(=O)NCc3ccc(OC)cc3)cc(C(=O)NCc3ccc(OC)cc3)c2)cc1] |
| M51 | 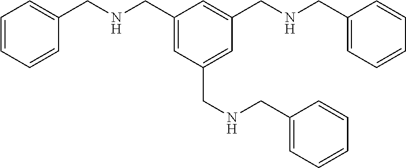 [SMILES: c1ccc(CNCc2cc(CNCc3ccccc3)cc(CNCc3ccccc3)c2)cc1] |
| M51S | 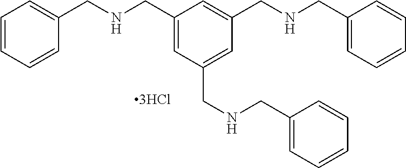 [SMILES: c1ccc(CNCc2cc(CNCc3ccccc3)cc(CNCc3ccccc3)c2)cc1] |
| M52 | 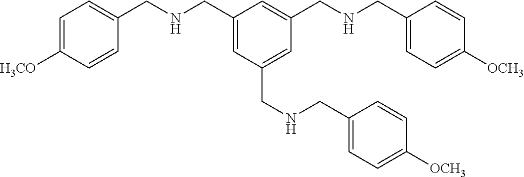 [SMILES: COc1ccc(CNCc2cc(CNCc3ccc(OC)cc3)cc(CNCc3ccc(OC)cc3)c2)cc1] |
| M53 | 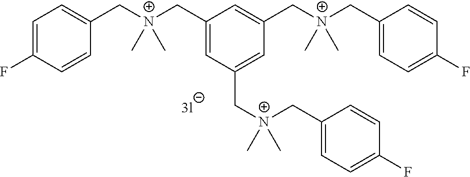 [SMILES: C[N+](C)(Cc1ccc(F)cc1)Cc1cc(C[N+](C)(C)Cc2ccc(F)cc2)cc(C[N+](C)(C)Cc2ccc(F)cc2)c1] |
| M54 | 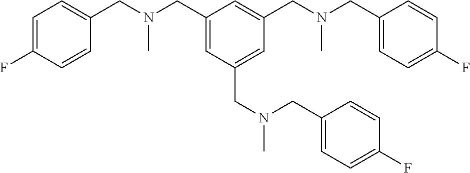 [SMILES: CN(Cc1ccc(F)cc1)Cc1cc(CN(C)Cc2ccc(F)cc2)cc(CN(C)Cc2ccc(F)cc2)c1] |
| M55 | 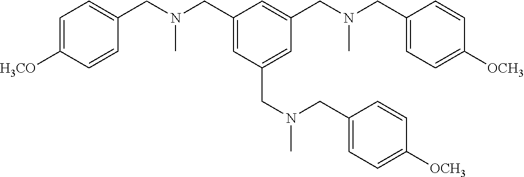 [SMILES: COc1ccc(CN(C)Cc2cc(CN(C)Cc3ccc(OC)cc3)cc(CN(C)Cc3ccc(OC)cc3)c2)cc1] |
| M56 | 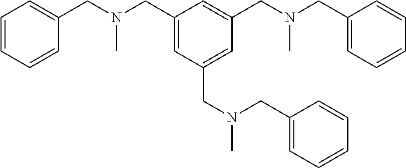 [SMILES: CN(Cc1ccccc1)Cc1cc(CN(C)Cc2ccccc2)cc(CN(C)Cc2ccccc2)c1] |
| M60 | 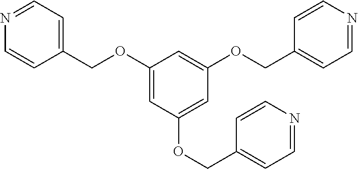 [SMILES: c1cc(COc2cc(OCc3ccncc3)cc(OCc3ccncc3)c2)ccn1] |
| M61 | 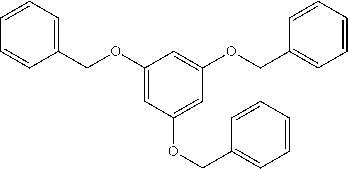 [SMILES: c1ccc(COc2cc(OCc3ccccc3)cc(OCc3ccccc3)c2)cc1] |
| M62 | 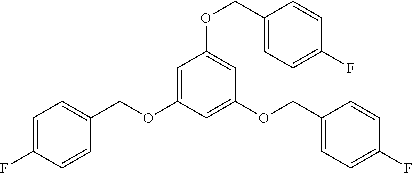 [SMILES: Fc1ccc(COc2cc(OCc3ccc(F)cc3)cc(OCc3ccc(F)cc3)c2)cc1] |
| M63 | 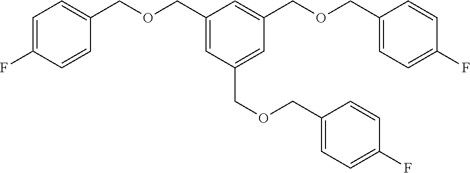 [SMILES: Fc1ccc(COCc2cc(COCc3ccc(F)cc3)cc(COCc3ccc(F)cc3)c2)cc1] |
| M64 | 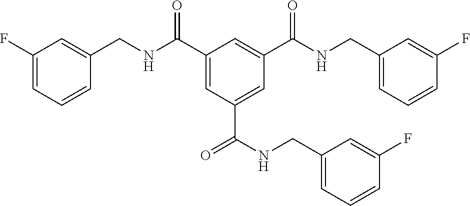 [SMILES: O=C(NCc1cccc(F)c1)c1cc(C(=O)NCc2cccc(F)c2)cc(C(=O)NCc2cccc(F)c2)c1] |
| M65 | 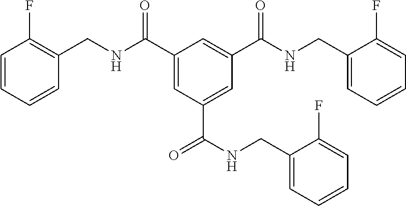 [SMILES: O=C(NCc1ccccc1F)c1cc(C(=O)NCc2ccccc2F)cc(C(=O)NCc2ccccc2F)c1] |
| M66 | 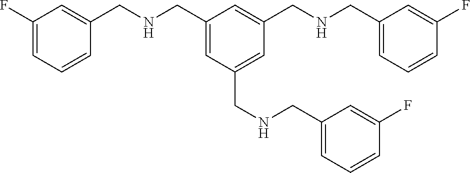 [SMILES: Fc1cccc(CNCc2cc(CNCc3cccc(F)c3)cc(CNCc3cccc(F)c3)c2)c1] |
| M67 | 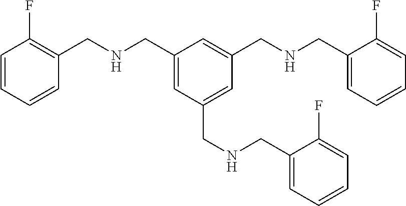 [SMILES: Fc1ccccc1CNCc1cc(CNCc2ccccc2F)cc(CNCc2ccccc2F)c1] |
| M68 | 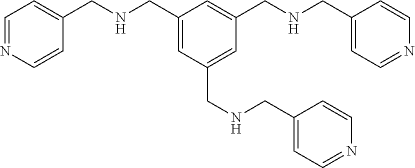 [SMILES: c1cc(CNCc2cc(CNCc3ccncc3)cc(CNCc3ccncc3)c2)ccn1] |
| M69 | 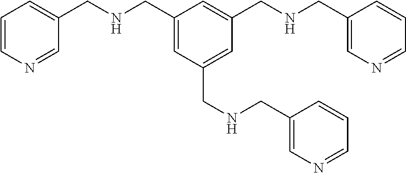 [SMILES: c1cncc(CNCc2cc(CNCc3cccnc3)cc(CNCc3cccnc3)c2)c1] |
| M69S | 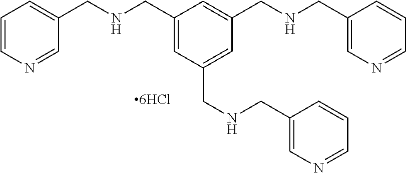 [SMILES: c1cncc(CNCc2cc(CNCc3cccnc3)cc(CNCc3cccnc3)c2)c1] |
| M70 | 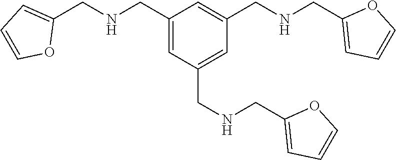 [SMILES: c1coc(CNCc2cc(CNCc3ccco3)cc(CNCc3ccco3)c2)c1] |
| M70S | 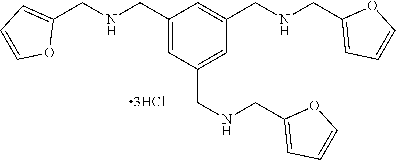 [SMILES: c1coc(CNCc2cc(CNCc3ccco3)cc(CNCc3ccco3)c2)c1] |
| M71 | 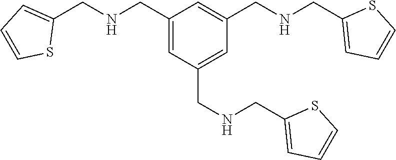 [SMILES: c1csc(CNCc2cc(CNCc3cccs3)cc(CNCc3cccs3)c2)c1] |
| M71S | 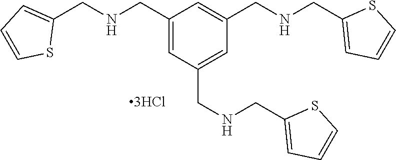 [SMILES: c1csc(CNCc2cc(CNCc3cccs3)cc(CNCc3cccs3)c2)c1] |
| M72S | 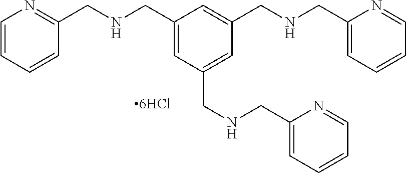 [SMILES: c1ccc(CNCc2cc(CNCc3ccccn3)cc(CNCc3ccccn3)c2)nc1] |
| M73 | 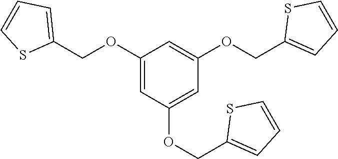 [SMILES: c1csc(COc2cc(OCc3cccs3)cc(OCc3cccs3)c2)c1] |
| M75 | 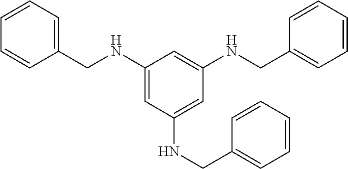 [SMILES: c1ccc(CNc2cc(NCc3ccccc3)cc(NCc3ccccc3)c2)cc1] |
| M75S | 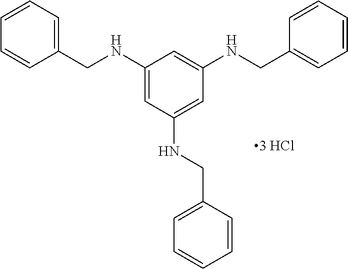 [SMILES: c1ccc(CNc2cc(NCc3ccccc3)cc(NCc3ccccc3)c2)cc1] |
| M76 | 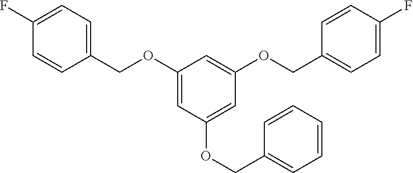 [SMILES: Fc1ccc(COc2cc(OCc3ccccc3)cc(OCc3ccc(F)cc3)c2)cc1] |
| M77 | 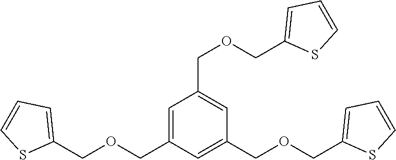 [SMILES: c1csc(COCc2cc(COCc3cccs3)cc(COCc3cccs3)c2)c1] |
| M78 | 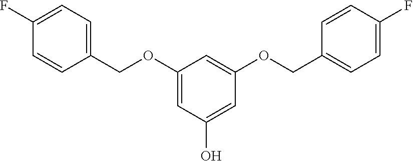 [SMILES: Oc1cc(OCc2ccc(F)cc2)cc(OCc2ccc(F)cc2)c1] |
| M79 | 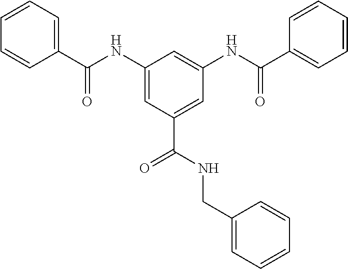 [SMILES: O=C(NCc1ccccc1)c1cc(NC(=O)c2ccccc2)cc(NC(=O)c2ccccc2)c1] |
| M80 | 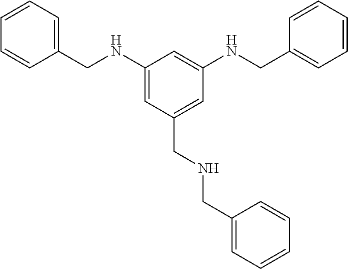 [SMILES: c1ccc(CNCc2cc(NCc3ccccc3)cc(NCc3ccccc3)c2)cc1] |
| M80S | 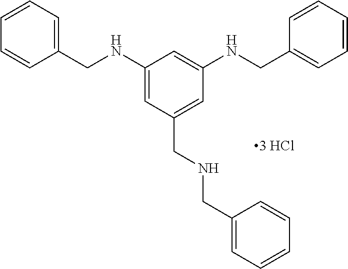 [SMILES: c1ccc(CNCc2cc(NCc3ccccc3)cc(NCc3ccccc3)c2)cc1] |
| M81 | 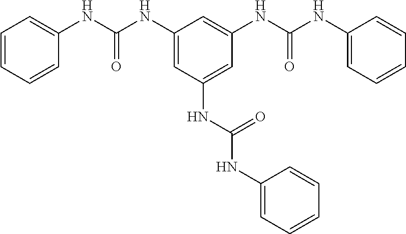 [SMILES: O=C(Nc1ccccc1)Nc1cc(NC(=O)Nc2ccccc2)cc(NC(=O)Nc2ccccc2)c1] |
| M82 | 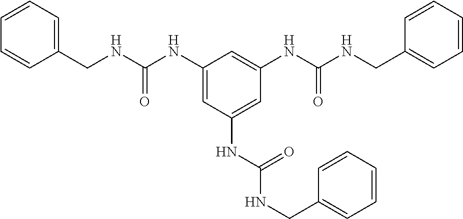 [SMILES: O=C(NCc1ccccc1)Nc1cc(NC(=O)NCc2ccccc2)cc(NC(=O)NCc2ccccc2)c1] |
| M83 | 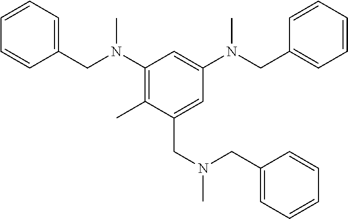 [SMILES: Cc1c(CN(C)Cc2ccccc2)cc(N(C)Cc2ccccc2)cc1N(C)Cc1ccccc1] |
| M84 | 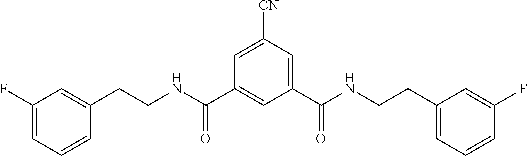 [SMILES: N#Cc1cc(C(=O)NCCc2cccc(F)c2)cc(C(=O)NCCc2cccc(F)c2)c1] |
| M85 | 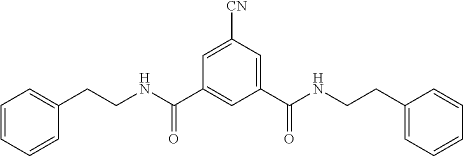 [SMILES: N#Cc1cc(C(=O)NCCc2ccccc2)cc(C(=O)NCCc2ccccc2)c1] |
| M86 | 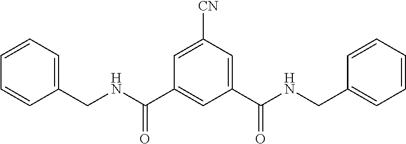 [SMILES: N#Cc1cc(C(=O)NCc2ccccc2)cc(C(=O)NCc2ccccc2)c1] |
| M87 | 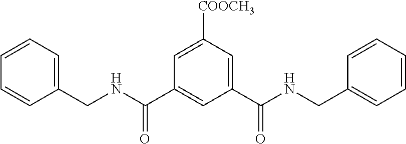 [SMILES: COC(=O)c1cc(C(=O)NCc2ccccc2)cc(C(=O)NCc2ccccc2)c1] |
| M89 | 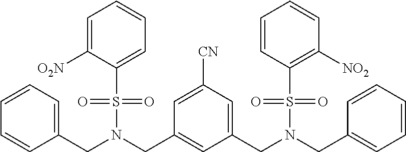 [SMILES: N#Cc1cc(CN(Cc2ccccc2)S(=O)(=O)c2ccccc2[N+](=O)[O-])cc(CN(Cc2ccccc2)S(=O)(=O)c2ccccc2[N+](=O)[O-])c1] |
| M90 | 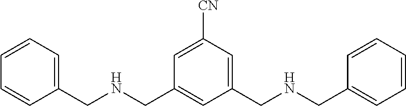 [SMILES: N#Cc1cc(CNCc2ccccc2)cc(CNCc2ccccc2)c1] |
| M91 | 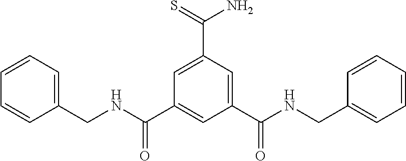 [SMILES: NC(=S)c1cc(C(=O)NCc2ccccc2)cc(C(=O)NCc2ccccc2)c1] |
| M92 | 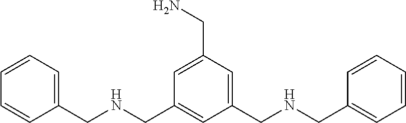 [SMILES: NCc1cc(CNCc2ccccc2)cc(CNCc2ccccc2)c1] |
| M93 | 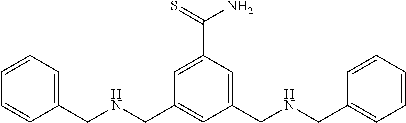 [SMILES: NC(=S)c1cc(CNCc2ccccc2)cc(CNCc2ccccc2)c1] |
| M94 | 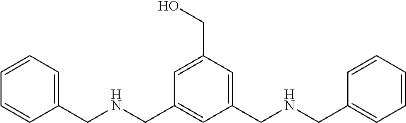 [SMILES: OCc1cc(CNCc2ccccc2)cc(CNCc2ccccc2)c1] |
| M95 | 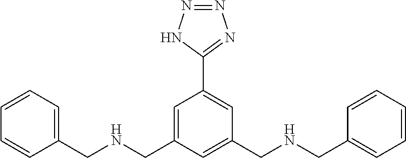 [SMILES: c1ccc(CNCc2cc(CNCc3ccccc3)cc(-c3nnn[nH]3)c2)cc1] |
| M96 | 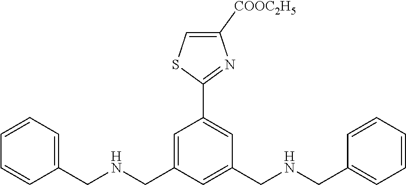 |
| M97 | 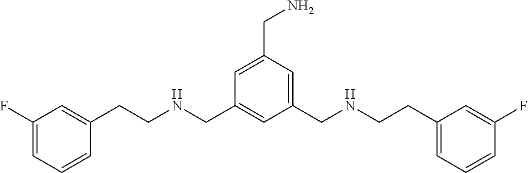 [SMILES: NCc1cc(CNCCc2cccc(F)c2)cc(CNCCc2cccc(F)c2)c1] |

### Example 3—Preparation of R20 and R20 Analogues of Class A

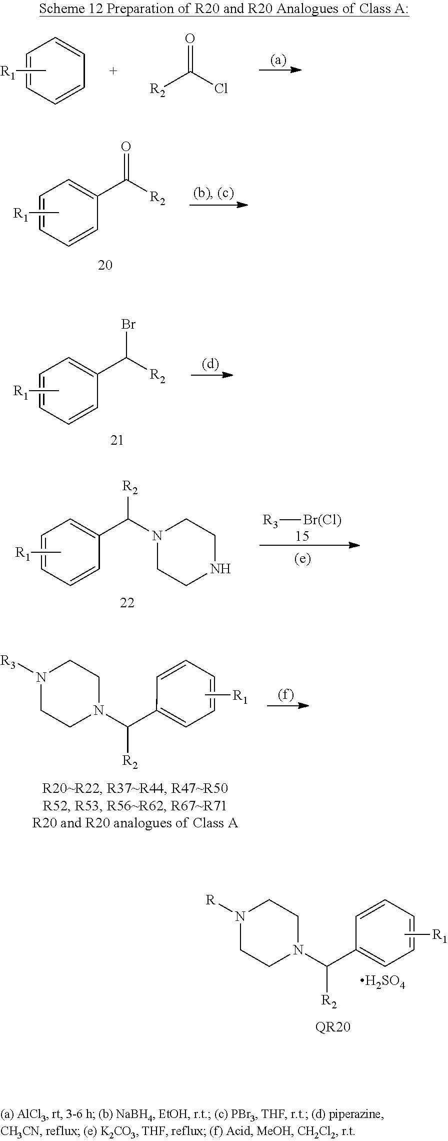

Compounds of R20 and R20 analogues of class A can be prepared by typical methods as illustrated in Scheme 12. Intermediates 20 were prepared according to the literatures [59,60], which were then converted to bromide 21 by reduction and then bromination. Subsequently substituted by piperazion, intermediates 22 were obtained. Treatment of intermediates 22 with halide 15 generated R20 and R20 Analogues of Class A: R20˜R22, R37˜R44, R47˜R50, R52, R53, R56˜R62.

General Procedure for the Preparation of Intermediates 21:

To a solution of 20 (53 mmol) in 80 mL EtOH, NaBH₄ (2 g, 53 mmol) was added at 0° C. The reaction mixture was stirred for 5 hours at r.t. After removing most of EtOH, the reaction mixture was acidified with diluted HCl and then extracted with EtOAc (3×40 mL). The combined organic layers were washed with brine (100 mL), dried over Na₂SO₄, and concentrated in vacuo. The crude residue was then dissolved in 40 mL dry CH₂Cl₂, PBr₃ (4.4 mL, 46.4 mmol) was added dropwise at 0° C. Then the resulting mixture was stirred for 1 hour at room temperature. Water was added and then extracted with CH₂Cl₂. The combined organic layers were washed with H₂O, saturated aqueous NaHCO₃, brine, dried over Na₂SO₄ and concentrated. The crude residue was purified by flash chromatography to give intermediates 21.

General Procedure for the Preparation of Intermediates 22:

A mixture of intermediates 21 (20 mmol) and piperazine (8.6 g, 100 mmol) in 100 mL acetonitrile was stirred under reflux for 11 hours. After cooling to r.t., the solvent was removed in vacuo. Water was added and extracted with EtOAc. The organic layers were washed with H₂O, dried over Na₂SO₄, filtered and concentrated. The crude residue was purified by flash chromatography to give intermediates 22.

General Procedure for the Preparation of R20 and R20 Analogues of Class A:

A mixture of intermediate 22 (1 mmol), halide 15 (3 mmol) and K₂CO₃ (10 mmol, 10 eq) in 25 mL THF was stirred overnight under reflux. After cooling to r.t., the mixture was filtered. The filtrate was concentrated and then purified by flash chromatography to generated R20 and R20 Analogues of Class A: R20˜R22, R37˜R44, R47˜R50, R52, R53, R56˜R62, R67˜R71.

General Procedure for the Preparation Sulfate Salt of R20 and R20 Analogues of Class A:

To a stirred solution of R20 (100 mg, 0.29 mmol) in 7 mL CH₂Cl₂, two drops of freshly prepared H₂SO₄: MeOH=1:4 (V/V) was added at room temperature. The reaction mixture was stirred overnight. Hexane was added to the mixture to generate solid from the solution. Cooled with ice-water bath for 2 hours. The crystals were collected by filtration to give QR20.

Characterization of R20 and R20 Analogues of Class A:

R20. Colorless syrup, yield: 87.3%. ¹H NMR (500 MHz, Acetone-d₆) δ 7.37-7.12 (m, 10H), 3.42 (s, 2H), 3.17 (d, J=9.4 Hz, 1H), 2.41 (brs, 8H), 2.10-2.13 (m, 1H), 2.03-1.95 (m, 1H), 1.74-1.77 (m, 1H), 1.66-1.55 (m, 2H), 1.46-1.42 (m, 1H), 1.34-1.26 (m, 1H), 1.23-1.08 (m, 2H), 0.97-0.84 (m, 1H), 0.82-0.78 (m, 1H). HRMS (ESI) m/z Found: 349.26514 [M+H]⁺, Calcd: 349.26437.

QR20: White solid. Yield 73.4%. ¹H NMR (500 MHz, D₂O) δ 7.55-7.34 (m, 10H), 4.22 (s, 2H), 4.05 (d, J=7.9 Hz, 1H), 3.75 (s, 1H), 3.38 (brs, 8H), 2.32-2.18 (m, 1H), 1.87-1.71 (m, 2H), 1.71-1.58 (m, 2H), 1.50-1.40 (m, 1H), 1.38-1.15 (m, 2H), 1.12-0.83 (m, 3H).

R21: Syrup, yield: 70.2%. ¹H NMR (500 MHz, CDCl₃) δ 7.52 (d, J=8.1 Hz, 2H), 7.38 (d, J=8.0 Hz, 2H), 7.33-7.19 (m, 3H), 7.14-7.09 (m, 2H), 3.49 (s, 2H), 3.10 (d, J=8.9 Hz, 1H), 2.42 (brs, 8H), 2.00 (d, J=13.2 Hz, 1H), 1.94-1.82 (m, 1H), 1.79-1.70 (m, 1H), 1.67-1.56 (m, 2H), 1.45 (d, J=13.3 Hz, 1H), 1.31-1.17 (m, 1H), 1.05-1.16 (m, 1H), 0.82-0.90 (m, 1H), 0.71-0.79 (m, 1H).

R22: Syrup, yield: 75.2%. ¹H NMR (500 MHz, CDCl₃) δ 7.26-7.29 (m, 2H), 7.24-7.17 (m, 3H), 7.13-7.08 (m, 2H), 6.93-6.96 (m, 2H), 3.40 (s, 2H), 3.09 (d, J=8.9 Hz, 1H), 2.40 (br, 8H), 2.01-1.98 (m, 1H), 1.90-1.88 (m, 1H), 1.73-1.75 (m, 1H), 1.66-1.55 (m, 2H), 1.46-1.43 (m, 1H), 1.34-1.26 (m, 1H), 1.23-1.08 (m, 2H), 0.86-0.83 (m, 1H), 0.77-0.73 (m, 1H)

R37: Syrup, yield: 45.3%. ¹H NMR (500 MHz, CDCl₃) δ 7.31-7.24 (m, 2H), 7.23-7.14 (m, 3H), 7.13-7.08 (m, 2H), 6.85-6.78 (m, 2H), 3.78 (s, 3H), 3.39 (s, 2H), 3.09 (d, J=8.9 Hz, 1H), 2.29 (brs, 8H), 2.00 (d, J=13.2 Hz, 1H), 1.94-1.84 (m, 1H), 1.74 (d, J=13.1 Hz, 1H), 1.67-1.52 (m, 2H), 1.45 (d, J=13.4 Hz, 1H), 1.31-1.00 (m, 3H), 0.93-0.69 (m, 2H).

R38: Syrup, yield: 70.0%. ¹H NMR (500 MHz, CDCl₃) δ 7.32-7.17 (m, 4H), 7.13-7.08 (m, 2H), 7.04-6.95 (m, 2H), 6.89 (td, J=8.2, 2.3 Hz, 1H), 3.43 (s, 2H), 3.09 (d, J=8.9 Hz, 1H), 2.41 (brs, 8H), 2.03-1.95 (m, 1H), 1.92-1.87 (m, 1H), 1.78-1.70 (m, 1H), 1.66-1.55 (m, 2H), 1.49-1.40 (m, 1H), 1.30-1.01 (m, 3H), 0.92-0.68 (m, 2H).

R39: Syrup, yield: 65.5%. ¹H NMR (500 MHz, CDCl₃) δ 7.33-7.23 (m, 3H), 7.23-7.16 (m, 2H), 7.13-7.08 (m, 2H), 7.05 (td, J=7.5, 1.1 Hz, 1H), 6.99 (ddd, J=9.5, 8.2, 1.1 Hz, 1H), 3.53 (s, 2H), 3.08 (d, J=8.9 Hz, 1H), 2.43 (brs, 8H), 2.03-1.95 (m, 1H), 1.93-1.83 (m, 1H), 1.78-1.69 (m, 1H), 1.65-1.55 (m, 2H), 1.51-1.39 (m, 1H), 1.30-1.04 (m, 3H), 0.90-0.68 (m, 2H).

R40: Syrup, yield: 57.6%. ¹H NMR (500 MHz, CDCl₃) δ 8.48-8.43 (m, 1H), 7.67 (td, J=7.7, 1.8 Hz, 1H), 7.40-7.31 (m, 3H), 7.30-7.14 (m, 4H), 3.56 (s, 2H), 3.19 (d, J=9.4 Hz, 1H), 2.47 (brs, 8H), 2.16-2.08 (m, 1H), 2.04-1.93 (m, 1H), 1.79-1.69 (m, 1H), 1.69-1.54 (m, 2H), 1.53-1.44 (m, 1H), 1.44-1.18 (m, 2H), 1.18-1.06 (m, 1H), 0.97-0.84 (m, 1H), 0.82-0.70 (m, 1H).

R41: Syrup, yield: 70.2%. ¹H NMR (500 MHz, CDCl₃) δ 7.31-7.18 (m, 5H), 7.10-7.03 (m, 2H), 7.00-6.92 (m, 2H), 3.45 (s, 2H), 3.07 (d, J=8.7 Hz, 1H), 2.42 (brs, 8H), 2.00-1.91 (m, 1H), 1.90-1.79 (m, 1H), 1.78-1.68 (m, 1H), 1.66-1.55 (m, 2H), 1.49-1.37 (m, 1H), 1.32-1.18 (m, 1H), 1.18-1.00 (m, 2H), 0.88-0.66 (m, 2H).

R42: Syrup, yield: 65.0%. ¹H NMR (500 MHz, CDCl₃) δ 7.42-7.30 (m, 1H), 7.24-7.16 (m, 2H), 7.10-7.03 (m, 2H), 7.01-6.90 (m, 3H), 3.41 (s, 2H), 3.07 (d, J=8.7 Hz, 1H), 2.39 (brs, 8H), 1.99-1.91 (m, 1H), 1.90-1.79 (m, 1H), 1.78-1.69 (m, 1H), 1.67-1.57 (m, 2H), 1.47-1.39 (m, 1H), 1.29-1.01 (m, 3H), 0.89-0.65 (m, 2H).

R43: Syrup, yield: 51.2%. ¹H NMR (500 MHz, CDCl₃) δ 8.50-8.45 (m, 2H), 7.63-7.57 (m, 1H), 7.33-7.18 (m, 4H), 7.14-7.09 (m, 2H), 3.46 (s, 2H), 3.10 (d, J=8.9 Hz, 1H), 2.43 (brs, 8H), 2.04-1.93 (m, 1H), 1.93-1.85 (m, 1H), 1.83-1.69 (m, 1H), 1.69-1.52 (m, 2H), 1.52-1.37 (m, 1H), 1.32-1.05 (m, 3H), 0.93-0.81 (m, 1H), 0.81-0.69 (m, 1H).

R44: Syrup, yield: 77.1%. ¹H NMR (500 MHz, CDCl₃) δ 7.31-7.14 (m, 4H), 7.13-7.07 (m, 2H), 6.86-6.79 (m, 2H), 6.79-6.72 (m, 1H), 3.77 (s, 3H), 3.42 (s, 2H), 3.08 (d, J=8.9 Hz, 1H), 2.41 (brs, 8H), 2.04-1.94 (m, 1H), 1.94-1.82 (m, 1H), 1.80-1.69 (m, 1H), 1.65-1.54 (m, 2H), 1.50-1.38 (m, 1H), 1.31-0.99 (m, 3H), 0.92-0.66 (m, 2H).

R47: Syrup, yield: 59.9%. ¹H NMR (500 MHz, CDCl₃) δ 7.57-7.52 (m, 2H), 7.37 (d, J=8.3 Hz, 2H), 7.32-7.19 (m, 3H), 7.13-7.07 (m, 2H), 3.48 (s, 2H), 3.10 (d, J=9.0 Hz, 1H), 2.41 (s, 8H), 2.04-1.94 (m, 1H), 1.93-1.83 (m, 1H), 1.78-1.70 (m, 1H), 1.65-1.55 (m, 2H), 1.51-1.35 (m, 1H), 1.27-1.04 (m, 3H), 0.92-0.80 (m, 1H), 0.79-0.68 (m, 1H).

R48: Syrup, yield: 80.2%. ¹H NMR (500 MHz, CDCl₃) δ 7.32-7.19 (m, 8H), 7.16-7.11 (m, 2H), 3.49 (s, 2H), 3.01 (d, J=8.7 Hz, 1H), 2.44 (s, 8H), 2.30-2.08 (m, 1H), 0.97 (d, J=6.6 Hz, 3H), 0.72 (d, J=6.6 Hz, 3H).

R49: Syrup, yield: 76.8%. ¹H NMR (500 MHz, CDCl₃) δ 7.42-7.14 (m, 10H), 3.51-3.40 (m, 2H), 3.10 (d, J=9.7 Hz, 1H), 2.85-2.66 (m, 1H), 2.38 (s, 8H), 2.22-2.07 (m, 1H), 1.98-1.86 (m, 1H), 1.84-1.62 (m, 2H), 1.52-1.36 (m, 2H).

R50: Syrup, yield: 66.9%. ¹H NMR (500 MHz, CDCl₃) δ 7.58 (s, 1H), 7.54-7.48 (m, 2H), 7.38 (t, J=7.7 Hz, 1H), 7.34-7.21 (m, 3H), 7.12 (d, J=7.0 Hz, 2H), 3.47 (s, 2H), 3.11 (d, J=8.9 Hz, 1H), 2.42 (brs, 8H), 2.06-1.97 (m, 1H), 1.96-1.85 (m, 1H), 1.80-1.71 (m, 1H), 1.67-1.58 (m, 2H), 1.50-1.40 (m, 1H), 1.32-1.02 (m, 3H), 0.94-0.81 (m, 1H), 0.81-0.69 (m, 1H).

R52: Syrup, yield: 66.2%. ¹H NMR (500 MHz, CDCl₃) δ 7.32-7.19 (m, 5H), 7.16-7.10 (m, 2H), 7.01-6.93 (m, 2H), 3.42 (s, 2H), 3.01 (d, J=8.7 Hz, 1H), 2.42-2.20 (m, 9H), 0.97 (d, J=6.6 Hz, 3H), 0.73 (d, J=6.6 Hz, 3H).

R53: Syrup, yield: 60.9%. ¹H NMR (500 MHz, CDCl₃) δ 7.31-7.17 (m, 7H), 7.01-6.93 (m, 2H), 3.47-3.37 (m, 2H), 3.12 (d, J=9.7 Hz, 1H), 2.81-2.69 (m, 1H), 2.38 (s, 8H), 2.18-2.11 (m, 1H), 1.99-1.87 (m, 1H), 1.84-1.63 (m, 2H), 1.53-1.39 (m, 2H).

R56: Syrup, yield: 56.2%. ¹H NMR (500 MHz, CDCl₃) δ 7.25-7.18 (m, 1H), 7.14-6.87 (m, 7H), 3.44 (s, 2H), 3.08 (d, J=8.6 Hz, 1H), 2.42 (s, 8H), 2.02-1.90 (m, 1H), 1.90-1.79 (m, 1H), 1.78-1.69 (m, 1H), 1.67-1.53 (m, 2H), 1.50-1.39 (m, 1H), 1.32-1.00 (m, 3H), 0.88-0.77 (m, 1H), 0.77-0.66 (m, 1H).

R57: Syrup, yield: 45.2%. ¹H NMR (500 MHz, CDCl₃) δ 7.34-7.28 (m, 1H), 7.26-7.19 (m, 1H), 7.11-6.93 (m, 6H), 3.55 (s, 2H), 3.08 (d, J=8.6 Hz, 1H), 2.70-2.12 (m, 8H), 2.01-1.92 (m, 1H), 1.91-1.78 (m, 1H), 1.77-1.69 (m, 1H), 1.68-1.56 (m, 2H), 1.50-1.40 (m, 1H), 1.32-1.01 (m, 3H), 0.90-0.64 (m, 2H).

R58: Syrup, yield: 52.7%. ¹H NMR (500 MHz, CDCl₃) δ 7.52 (d, J=8.1 Hz, 2H), 7.38 (d, J=8.0 Hz, 2H), 7.14-7.02 (m, 2H), 7.01-6.94 (m, 2H), 3.49 (s, 2H), 3.08 (d, J=8.7 Hz, 1H), 2.42 (s, 8H), 2.01-1.92 (m, 1H), 1.90-1.79 (m, 1H), 1.78-1.69 (m, 1H), 1.67-1.56 (m, 2H), 1.49-1.39 (m, 1H), 1.30-1.00 (m, 3H), 0.90-0.65 (m, 2H).

R59: Syrup, yield: 68.8%. ¹H NMR (500 MHz, CDCl₃) δ 7.38-7.17 (m, 5H), 7.15-7.05 (m, 3H), 3.38 (s, 2H), 3.09 (d, J=8.9 Hz, 1H), 2.40 (brs, 8H), 2.04-1.94 (m, 1H), 1.94-1.82 (m, 1H), 1.79-1.68 (m, 1H), 1.67-1.53 (m, 2H), 1.50-1.38 (m, 1H), 1.33-1.00 (m, 3H), 0.93-0.79 (m, 1H), 0.80-0.68 (m, 1H).

R60: Syrup, yield: 64.8%. ¹H NMR (500 MHz, CDCl₃) δ 7.34-7.25 (m, 2H), 7.25-7.16 (m, 1H), 7.15-7.01 (m, 4H), 6.99-6.93 (m, 1H), 3.39 (s, 2H), 3.11 (d, J=8.9 Hz, 1H), 2.41 (brs, 8H), 2.06-1.96 (m, 1H), 1.96-1.85 (m, 1H), 1.81-1.71 (m, 1H), 1.68-1.54 (m, 2H), 1.50-1.41 (m, 1H), 1.33-1.02 (m, 3H), 0.93-0.81 (m, 1H), 0.81-0.70 (m, 1H).

R61: Syrup, yield: 53.9%. ¹H NMR (500 MHz, CDCl₃) δ 7.33-7.16 (m, 3H), 7.14-7.06 (m, 2H), 6.43 (d, J=1.8 Hz, 2H), 6.32 (t, J=2.2 Hz, 1H), 3.75 (s, 6H), 3.39 (s, 2H), 3.09 (d, J=8.8 Hz, 1H), 2.41 (brs, 8H), 2.05-1.94 (m, 1H), 1.94-1.81 (m, 1H), 1.80-1.68 (m, 1H), 1.68-1.52 (m, 2H), 1.50-1.39 (m, 1H), 1.31-0.98 (m, 3H), 0.93-0.65 (m, 2H).

R62: Syrup, yield: 64.3%. ¹H NMR (500 MHz, CDCl₃) δ 7.11-7.04 (m, 2H), 7.00-6.93 (m, 2H), 6.83-6.75 (m, 3H), 3.85 (s, 6H), 3.39 (s, 2H), 3.07 (d, J=8.6 Hz, 1H), 2.38 (brs, 8H), 2.00-1.91 (m, 1H), 1.90-1.80 (m, 1H), 1.78-1.70 (m, 1H), 1.66-1.56 (m, 2H), 1.49-1.40 (m, 1H), 1.31-1.00 (m, 3H), 0.87-0.77 (m, 1H), 0.77-0.67 (m, 1H).

R67: Colorless syrup, 40.9% yield. ¹H NMR (500 MHz, CDCl₃) δ 7.31-7.24 (m, 2H), 7.23-7.17 (m, 2H), 7.13-7.08 (m, 2H), 6.94-6.84 (m, 2H), 3.66 (s, 2H), 3.08 (d, J=8.8 Hz, 1H), 2.46 (brs, 8H), 2.02-1.82 (m, 2H), 1.79-1.68 (m, 1H), 1.67-1.55 (m, 2H), 1.50-1.41 (m, 1H), 1.31-1.00 (m, 3H), 0.91-0.67 (m, 2H).

R68: White solid, 60.4% yield. ¹H NMR (500 MHz, Acetone-d₆) δ 7.37-7.30 (m, 2H), 7.29-7.10 (m, 8H), 3.17 (d, J=9.2 Hz, 1H), 2.74-2.67 (m, 2H), 2.63-2.18 (m, 10H), 2.16-1.96 (m, 2H), 1.80-1.71 (m, 1H), 1.68-1.57 (m, 2H), 1.51-1.42 (m, 1H), 1.37-1.05 (m, 3H), 0.95-0.84 (m, 1H), 0.83-0.71 (m, 1H).

R69: Colorless syrup, 70.2% yield. ¹H NMR (500 MHz, CDCl₃) δ 7.39-7.22 (m, 8H), 7.12-7.07 (m, 2H), 3.74 (d, J=47.0 Hz, 2H), 3.37 (s, 2H), 3.14 (d, J=9.2 Hz, 1H), 2.68-2.12 (m, 4H), 2.10-1.97 (m, 1H), 1.95-1.83 (m, 1H), 1.81-1.70 (m, 1H), 1.69-1.54 (m, 2H), 1.48-1.37 (m, 1H), 1.33-1.02 (m, 3H), 0.99-0.83 (m, 1H), 0.83-0.68 (m, 1H).

R70: syrup, 65.5% yield. ¹H NMR (500 MHz, CDCl₃) δ 7.95 (d, J=7.3 Hz, 2H), 7.54 (t, J=7.4 Hz, 1H), 7.42 (t, J=7.7 Hz, 2H), 7.32-7.24 (m, 2H), 7.24-7.18 (m, 1H), 7.13 (d, J=7.1 Hz, 2H), 3.76 (s, 2H), 3.13 (d, J=8.7 Hz, 1H), 2.72-2.30 (m, 8H), 2.05-1.84 (m, 2H), 1.79-1.70 (m, 1H), 1.66-1.56 (m, 2H), 1.53-1.41 (m, 1H), 1.33-1.01 (m, 3H), 0.92-0.69 (m, 2H).

R71: syrup, 53.3% yield. ¹H NMR (500 MHz, CDCl₃) δ 7.33-7.19 (m, 4H), 7.14-7.09 (m, 2H), 6.42 (d, J=3.6 Hz, 1H), 3.59 (s, 2H), 3.10 (d, J=8.9 Hz, 1H), 2.66-2.23 (m, 8H), 2.04-1.95 (m, 1H), 1.95-1.85 (m, 1H), 1.80-1.71 (m, 1H), 1.67-1.60 (m, 2H), 1.51-1.41 (m, 1H), 1.31-1.03 (m, 3H), 0.93-0.81 (m, 1H), 0.81-0.71 (m, 1H).

TABLE 3
Structures of R20 and R20 Analogues of Class A.

| ID | Structure |
|---|---|
| R20 | 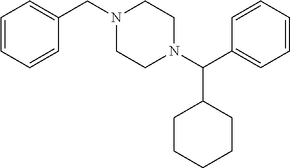 |
| QR20 | 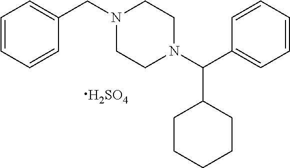 |
| R21 | 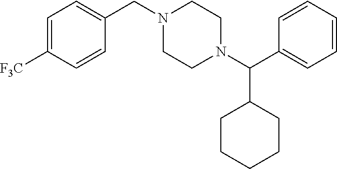 |
| R22 | 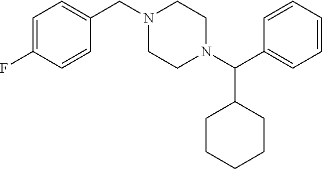 |
| R37 | 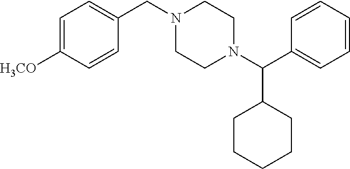 |
| R38 | 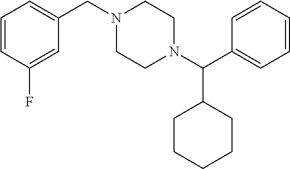 |
| R39 | 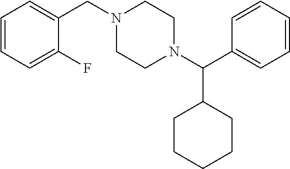 |
| R40 | 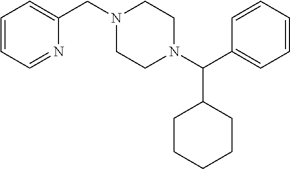 |
| R41 | 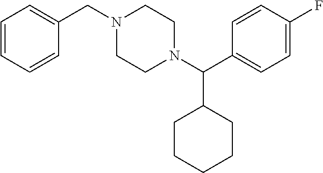 |
| R42 | 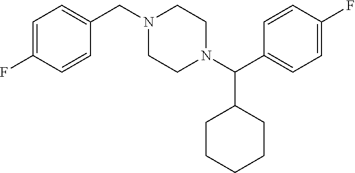 |
| R43 | 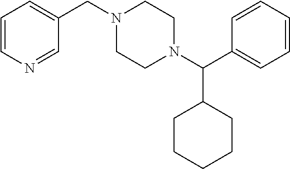 |
| R44 | 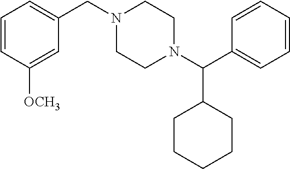 |
| R47 | 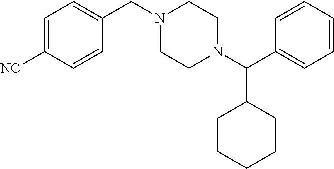 |
| R48 | 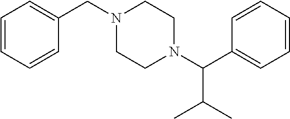 |
| R49 | 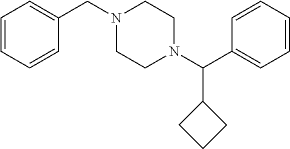 |
| R50 | 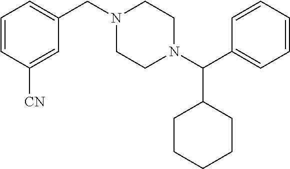 |
| R52 | 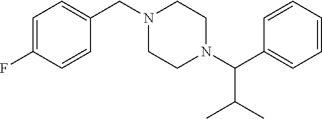 |
| R53 | 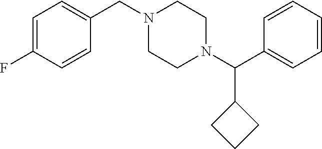 |
| R56 | 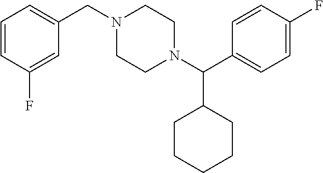 |
| R57 | 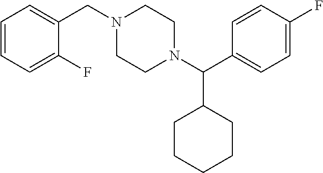 |
| R58 | 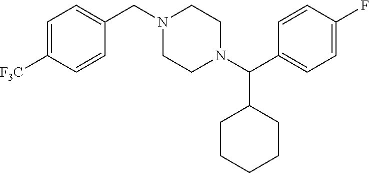 |
| R59 | 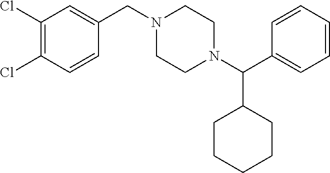 |
| R60 | 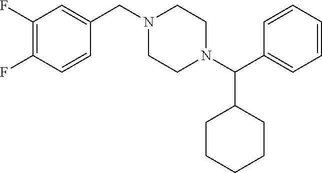 |
| R61 | 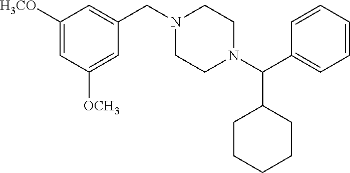 |
| R62 | 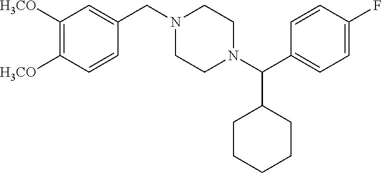 |
| R67 | 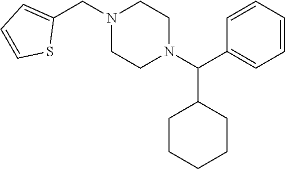 |
| R68 | 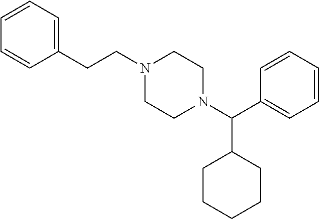 |
| R69 | 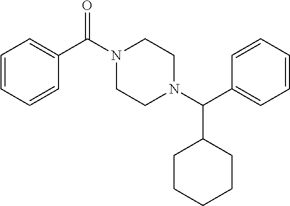 |
| R70 | 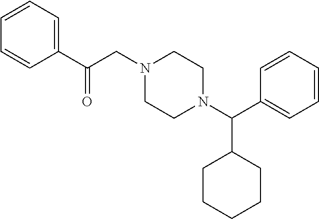 |
| R71 | 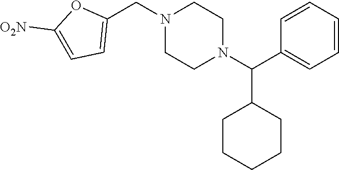 |

### Example 4—Preparation of R20 Analogues of Class B

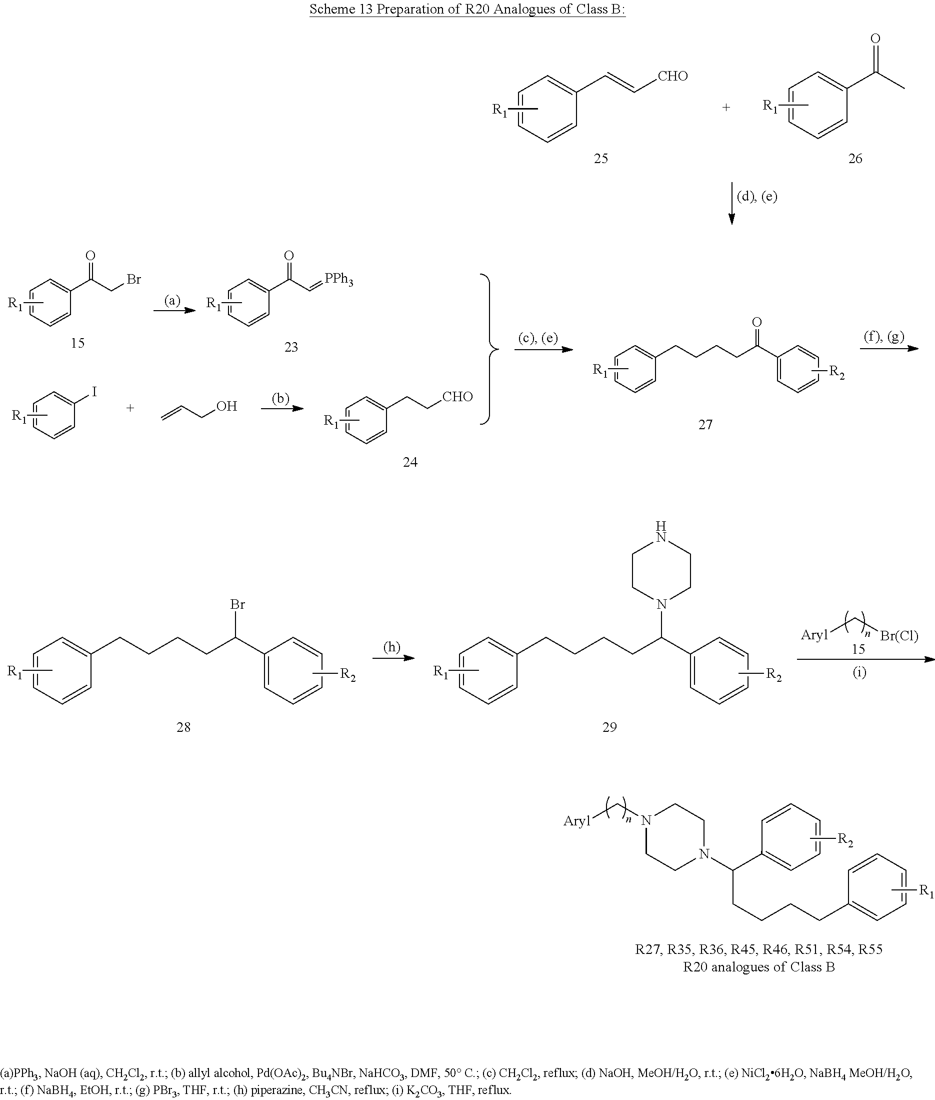

Compounds of R20 analogues of class B can be prepared by typical methods as illustrated in Scheme 13. Intermediates 27 were prepared according to the literatures [60-62]. Similarly, as illustrated in Scheme 6, by reduction and then bromination, intermediates 27 were converted to bromide 28. Subsequently substituted by piperazion, intermediates 23 were obtained. Treatment of intermediates 29 with halides 15 generated R20 Analogues of Class B: R27, R35, R36, R45, R46, R51, R54, R55.

General Procedure for the Preparation of Intermediates 22:

Intermediates 22 were prepared by generally following the procedure as described above for intermediates 13.

General Procedure for the Preparation of Intermediates 23:

Intermediates 23 were prepared by generally following the procedure as described above for intermediates 14.

General Procedure for the Preparation of R20 Analogues of Class B:

A mixture of intermediate 23 (1 mmol), halides 15 (3 mmol) and K₂CO₃ (10 mmol, 10 eq) in 25 mL THF was stirred overnight under reflux. After cooling to r.t., the mixture was filtered. The filtrate was concentrated and then purified by flash chromatography to generated R20 Analogues of Class B: R27, R35, R36, R45, R46, R51, R54, R55.

Characterization of R20 Analogues of Class B:

R27: Syrup. ¹H NMR (500 MHz, CDCl₃) δ 7.36-7.26 (m, 3H), 7.25-7.08 (m, 9H), 6.99-6.92 (m, 2H), 3.42 (s, 2H), 3.29-3.22 (m, 1H), 2.71-2.17 (m, 10H), 1.95-1.85 (m, 1H), 1.81-1.70 (m, 1H), 1.60-1.48 (m, 2H), 1.24-1.03 (m, 2H).

R35: Syrup. ¹H NMR (500 MHz, CDCl₃) δ 7.32-7.07 (m, 15H), 3.46 (s, 2H), 3.31-3.21 (m, 1H), 2.71-2.18 (m, 10H), 1.97-1.85 (m, 1H), 1.83-1.68 (m, 1H), 1.63-1.48 (m, 2H), 1.28-1.18 (m, 1H), 1.16-1.05 (m, 1H). MS (ESI) m/z Found: 399.28 [M+H]⁺, Calcd: 399.28.

R36: Syrup. ¹H NMR (500 MHz, CDCl₃) δ 7.31-7.11 (m, 7H), 7.08-6.90 (m, 4H), 6.84-6.75 (m, 2H), 3.77 (s, 3H), 3.46 (s, 2H), 3.29-3.18 (m, 1H), 2.75-2.09 (m, 10H), 1.96-1.83 (m, 1H), 1.76-1.64 (m, 1H), 1.57-1.43 (m, 2H), 1.23-1.12 (m, 1H), 1.11-0.98 (m, 1H). MS (ESI) m/z Found: 447.28 [M+H]⁺, Calcd: 447.28.

R45: Syrup. ¹H NMR (500 MHz, CDCl₃) δ 7.40-7.13 (m, 7H), 7.06-6.95 (m, 4H), 6.82-6.76 (m, 2H), 3.77 (s, 3H), 3.30-3.19 (m, 1H), 2.81-2.71 (m, 2H), 2.70-2.20 (m, 12H), 1.97-1.84 (m, 1H), 1.76-1.62 (m, 1H), 1.60-1.44 (m, 2H), 1.22-1.02 (m, 2H).

R46: Syrup. ¹H NMR (500 MHz, CDCl₃) δ 7.23-7.09 (m, 4H), 7.06-6.90 (m, 6H), 6.82-6.76 (m, 2H), 3.77 (s, 3H), 3.31-3.18 (m, 1H), 2.78-2.67 (m, 2H), 2.67-2.17 (m, 12H), 1.98-1.82 (m, 1H), 1.79-1.64 (m, 1H), 1.60-1.45 (m, 2H), 1.23-0.99 (m, 2H).

R51: Syrup. ¹H NMR (500 MHz, CDCl₃) δ 7.33-7.15 (m, 7H), 7.07-7.00 (m, 2H), 6.99-6.87 (m, 4H), 3.42 (s, 2H), 3.29-3.20 (m, 1H), 2.69-2.10 (m, 10H), 1.94-1.83 (m, 1H), 1.81-1.69 (m, 1H), 1.61-1.43 (m, 2H), 1.26-1.15 (m, 1H), 1.15-1.01 (m, 1H).

R54: Syrup. ¹H NMR (500 MHz, CDCl₃) δ 7.34-7.17 (m, 6H), 7.15-7.08 (m, 2H), 7.07-7.00 (m, 2H), 6.98-6.88 (m, 3H), 3.29-3.21 (m, 1H), 2.77-2.68 (m, 2H), 2.61-2.26 (m, 12H), 1.97-1.85 (m, 1H), 1.83-1.67 (m, 1H), 1.63-1.44 (m, 2H), 1.24-1.16 (m, 1H), 1.14-1.03 (m, 1H).

R55: Syrup. ¹H NMR (500 MHz, CDCl₃) δ 7.32-7.15 (m, 9H), 7.06-6.99 (m, 2H), 6.95-6.87 (m, 2H), 3.50-3.41 (m, 2H), 3.28-3.21 (m, 1H), 2.62-2.22 (m, 10H), 1.94-1.83 (m, 1H), 1.81-1.71 (m, 1H), 1.58-1.45 (m, 2H), 1.22-1.05 (m, 2H).

TABLE 4
Structure of R20 Analogues of Class B.

| | |
|---|---|
| R27 | 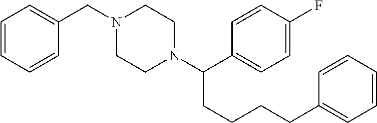 |
| R35 | 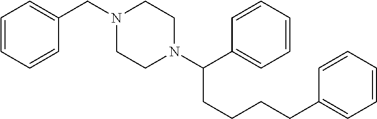 |
| R36 | 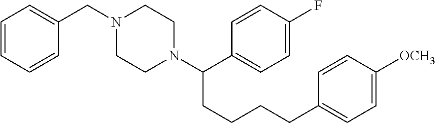 |
| R45 | 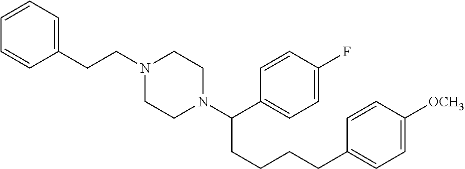 |
| R46 | 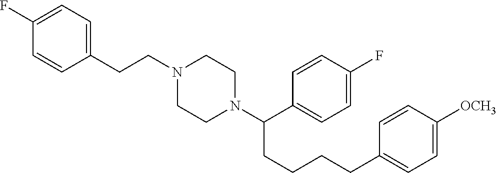 |
| R51 | 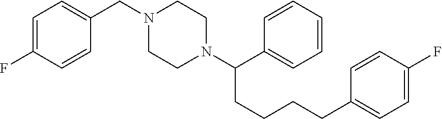 |
| R54 | 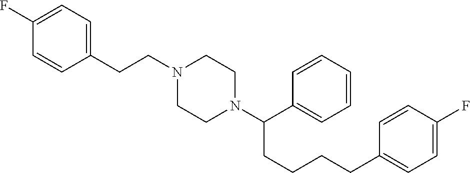 |
| R55 | 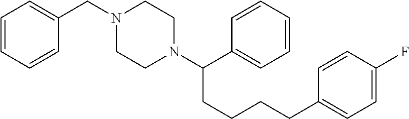 |

### Example 5—Preparation of R20 Analogues of Class C and Class D

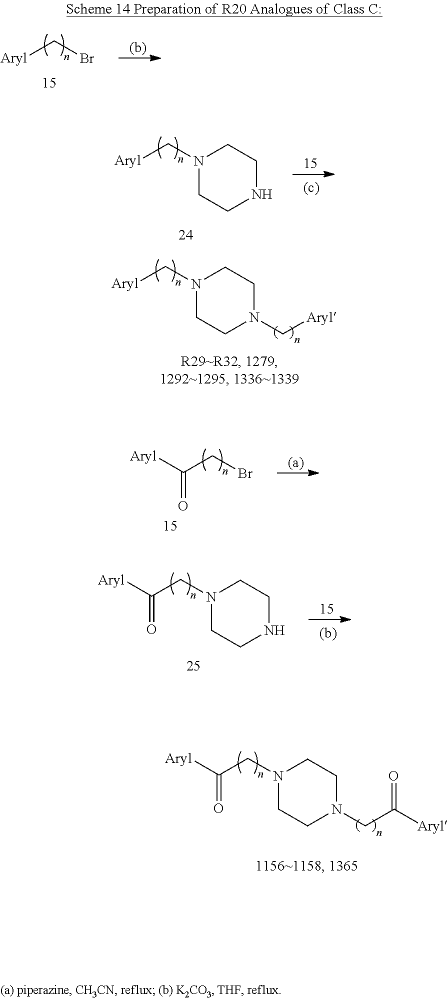

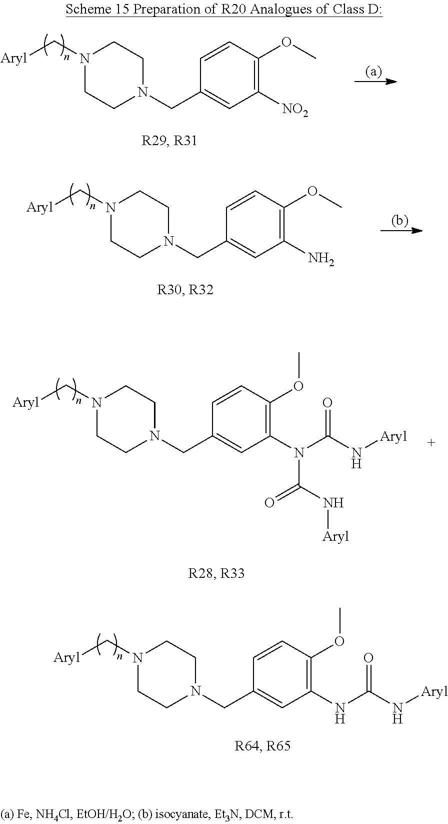

Compounds of R20 analogues of class C and class D can be prepared by typical methods as illustrated in Scheme 14 and scheme 15. Intermediates 30 and 31 were prepared by the typical procedure as described above for intermediates 21. By the alkylation of 30 or 31, compounds of R20 analogues of class C: R29˜R32, 1292˜1295, 1336˜1339 were obtained. As illustrated in Scheme 9, by the reduction of some of the R20 analogues of class C, amines R30 and R32 were obtained. Subsequently reacted with isocyanate generated R20 Analogues of Class D: R28, R33, R64, R65.

General Procedure for the Preparation of Intermediates 30 and 31:

Intermediates 30 and 31 were prepared by generally following the procedure as described above for intermediates 21.

General Procedure for the Preparation of R20 Analogues of Class C:

A mixture of intermediate 30 or 31 (1 mmol), halide 15 (3 mmol) and K₂CO₃ (10 mmol, 10 eq) in 25 mL THF was stirred overnight under reflux. After cooling to r.t., the mixture was filtered. The filtrate was concentrated and then purified by flash chromatography to generated R20 Analogues of Class C: R29˜R32, 1279, 1292˜1295, 1336˜1339, 1156˜1158, 1365.

General Procedure for the Preparation of R30 and R32:

A mixture of R29 or R31 (1 eq, 1 mmol), Fe (20 eq, 20 mmol), NH₄Cl (0.5 mmol), and H₂O (2.5 mL) in 10 mL EtOH was heated under reflux for 1.5 hour. The reaction mixture was cooled and filtered. The filtrate was concentrated and the residue was purified by flash chromatography to give the desired products R30 and R32.

General Procedure for the Preparation of R28 Analogues of Class D:

A mixture of R30 or R32 (34 mg, 0.1 mmol), 1-fluoro-3-isocyanatobenzene (30 mg, 0.22 mmol) and Et₃N (4 drops) in 10 mL CH₂Cl₂ was stirred at r.t. overnight. The reaction mixture was concentrated and purified by chromatography to give the desired compound R28, R30, R64, R65.

Characterization of R20 Analogues of Class C and Class D:

R28 as light yellow solid (10 mg, 16.2%). ¹H NMR (500 MHz, Acetone-d₆) δ 10.08 (s, 1H), 9.06 (s, 1H), 8.11 (s, 1H), 7.72-7.64 (m, 1H), 7.58-7.44 (m, 3H), 7.42-7.32 (m, 2H), 7.29-7.18 (m, 3H), 7.05-6.99 (m, 1H), 6.94 (d, J=8.3 Hz, 1H), 6.91-6.84 (m, 3H), 3.79 (s, 3H), 3.78 (s, 3H), 3.42 (s, 2H), 3.41 (s, 2H), 2.41 (brs, 8H). HRMS (ESI) m/z Found: 616.2742 [M+H]⁺, Calcd: 616.2735.

R29: yellow solid, 91.2% yield. ¹H NMR (500 MHz, CDCl₃) δ 7.80 (d, J=2.2 Hz, 1H), 7.48 (dd, J=8.6, 2.2 Hz, 1H), 7.21 (d, J=8.6 Hz, 2H), 7.02 (d, J=8.6 Hz, 1H), 6.88-6.81 (m, 2H), 3.94 (s, 3H), 3.79 (s, 3H), 3.47 (s, 2H), 3.45 (s, 2H), 2.45 (brs, 8H).

R30: white solid, 82.8%. ¹H NMR (500 MHz, CDCl₃) δ 7.23 (d, J=8.6 Hz, 2H), 6.88-6.82 (m, 2H), 6.71 (d, J=8.1 Hz, 2H), 6.64 (dd, J=8.2, 1.9 Hz, 1H), 3.84 (s, 3H), 3.80 (s, 3H), 3.76 (s, 2H), 3.47 (s, 2H), 3.39 (s, 2H), 2.47 (brs, 8H).

R31: yellow oil, 88.3% yield. ¹H NMR (500 MHz, CDCl₃) δ 8.02 (dd, J=7.2, 2.2 Hz, 1H), 7.62-7.55 (m, 1H), 7.25-7.18 (m, 3H), 6.88-6.81 (m, 2H), 3.80 (s, 3H), 3.52 (s, 2H), 3.46 (s, 2H), 2.46 (brs, 8H).

R32: light yellow solid, 75.0%. ¹H NMR (500 MHz, CDCl₃) δ 7.21 (d, J=8.6 Hz, 2H), 6.92-6.81 (m, 3H), 6.75 (dd, J=8.8, 1.9 Hz, 1H), 6.63-6.56 (m, 1H), 3.79 (s, 3H), 3.66 (brs, 2H), 3.44 (s, 2H), 3.36 (s, 2H), 2.44 (brs, 8H).

R33: White solid, 20.3% yield. ¹H NMR (500 MHz, CDCl₃) δ 9.65 (brs, 1H), 8.32 (brs, 1H), 7.98-7.92 (m, 1H), 7.62-7.57 (m, 1H), 7.40 (m, 1H), 7.32-7.18 (m, 6H), 7.13-6.96 (m, 3H), 6.88-6.78 (m, 3H), 3.79 (s, 3H), 3.45 (d, J=7.4 Hz, 4H), 2.45 (brs, 8H).

R64: Colorless syrup, 80.2% yield. ¹H NMR (500 MHz, CDCl₃) δ 8.48 (s, 1H), 8.43 (s, 1H), 7.67-7.57 (m, 2H), 7.51-7.43 (m, 1H), 7.25-7.19 (m, 2H), 7.10-7.02 (m, 1H), 6.89-6.80 (m, 3H), 3.92 (s, 3H), 3.79 (s, 3H), 3.52 (s, 2H), 3.46 (s, 2H), 2.49 (brs, 8H).

R65: Colorless syrup, 50.2% yield. ¹H NMR (500 MHz, CDCl₃) δ 8.25 (d, J=1.8 Hz, 1H), 7.76 (brs, 1H), 7.35 (dd, J=14.0, 7.9 Hz, 1H), 7.20 (d, J=8.5 Hz, 2H), 7.13 (d, J=7.5 Hz, 1H), 7.10-7.05 (m, 1H), 7.05-6.94 (m, 2H), 6.83 (d, J=8.6 Hz, 2H), 6.76 (d, J=8.3 Hz, 1H), 3.79 (s, 3H), 3.76 (s, 3H), 3.73 (s, 2H), 3.44 (d, J=5.5 Hz, 4H), 2.45 (brs, 8H).

R66: Colorless syrup, 69.4% yield. ¹H NMR (500 MHz, Acetone-d₆) δ 8.91 (brs, 1H), 8.28 (s, 1H), 7.93 (brs, 1H), 7.64 (dt, J=12.0, 2.3 Hz, 1H), 7.33-7.21 (m, 3H), 7.21-7.15 (m, 1H), 6.92 (d, J=0.9 Hz, 2H), 6.90-6.84 (m, 2H), 6.78-6.70 (m, 1H), 3.87 (s, 3H), 3.78 (s, 3H), 3.42 (d, J=5.7 Hz, 4H), 2.43 (brs, 8H). HRMS (ESI) m/z Found: 479.2459 [M+H]+, Calcd: 479.2453.

1279: ¹H NMR (500 MHz, CDCl₃) δ 8.62-8.53 (m, 2H), 7.73-7.59 (m, 2H), 7.41 (d, J=7.8 Hz, 2H), 7.17-7.15 (m, 2H), 3.68 (s, 4H), 2.59 (br, 8H).

1292: ¹H NMR (500 MHz, CDCl₃) δ 7.24-7.18 (m, 4H), 6.86-6.81 (m, 4H), 3.79 (s, 6H), 3.44 (s, 4H), 2.45 (br, 8H), 2.45 (br, 8H).

1293: ¹H NMR (500 MHz, CDCl₃) δ 7.25-7.18 (m, 2H), 6.94-6.87 (m, 4H), 6.81-6.76 (m, 2H), 3.80 (s, 6H), 3.49 (s, 4H), 2.48 (br, 8H)

1294: ¹H NMR (500 MHz, CDCl₃) δ 7.27-7.23 (m, 2H), 7.08-7.05 (m, 4H), 6.95-6.90 (m, 2H), 3.50 (s, 4H), 2.48 (br, 8H).

1295: White solid. ¹H NMR (500 MHz, CDCl₃) δ 7.32-7.24 (m, 4H), 7.05-6.94 (m, 4H), 3.48 (s, 4H), 2.46 (br, 8H).

1336: White solid. ¹H NMR (500 MHz, CDCl₃) δ 7.36-733 (m, 2H), 7.24-7.20 (m, 2H), 7.10-7.07 (m, 2H), 7.03-6.99 (m, 2H), 3.60 (s, 4H), 2.53 (br, 8H).

1337: White solid. ¹H NMR (500 MHz, CDCl₃) δ 7.66 (s, 2H), 7.57-7.52 (m, 4H), 7.41 (t, J=7.7 Hz, 2H), 3.53 (s, 4H), 2.47 (br, 8H).

1338: White solid. ¹H NMR (500 MHz, CDCl₃) δ 7.64-7.63 (d, J=7.6 Hz, 2H), 7.55-7.54 (m, 4H), 7.37-7.31 (m, 2H), 3.71 (s, 4H), 2.55 (br, 8H).

1339: White solid. ¹H NMR (500 MHz, CDCl₃) δ 7.60 (d, J=8.3 Hz, 4H), 7.44 (d, J=8.3 Hz, 4H), 3.55 (s, 4H), 2.47 (br, 8H).

1365: White solid. ¹H NMR (500 MHz, CDCl₃) δ 8.01-7.99 (m, 4H), 7.61-7.52 (m, 2H), 7.53-7.40 (m, 4H), 3.86 (s, 4H), 2.73 (br, 8H).

TABLE 5
Structures of R20 Analogues of Class C.

| ID | Structure |
|---|---|
| R28 | 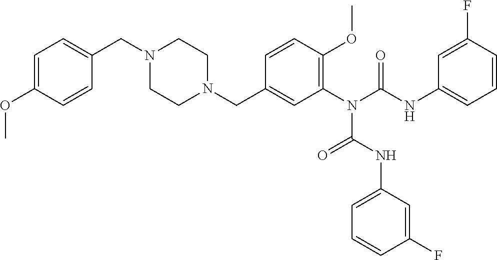 |
| R29 | 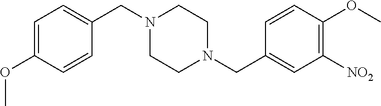 |
| R30 | 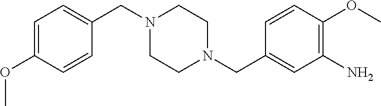 |
| R31 | 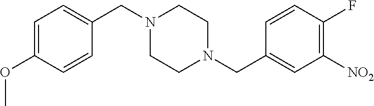 |
| R32 | 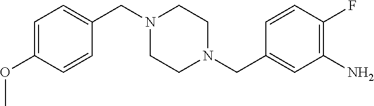 |
| R33 | 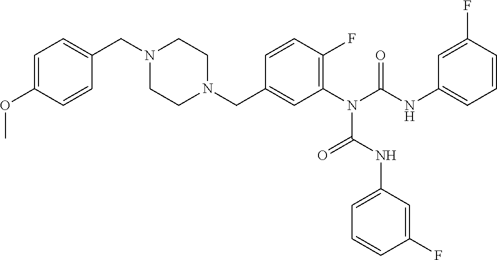 |
| R64 | 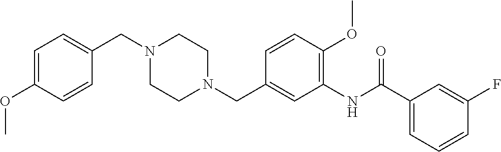 |
| R65 | 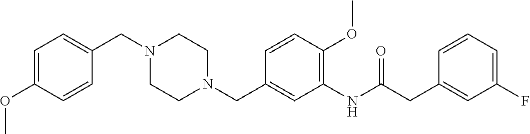 |
| R66 | 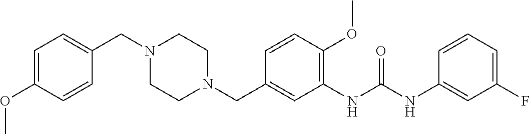 |
| 1156 | 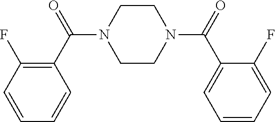 [SMILES: O=C(c1ccccc1F)N1CCN(C(=O)c2ccccc2F)CC1] |
| 1157 | 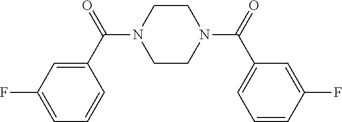 [SMILES: O=C(c1cccc(F)c1)N1CCN(C(=O)c2cccc(F)c2)CC1] |
| 1158 | 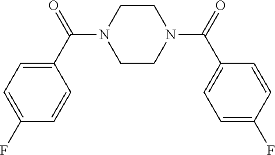 [SMILES: O=C(c1ccc(F)cc1)N1CCN(C(=O)c2ccc(F)cc2)CC1] |
| 1279 | 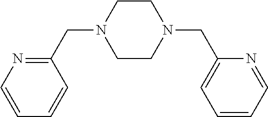 [SMILES: c1ccc(CN2CCN(Cc3ccccn3)CC2)nc1] |
| 1292 | 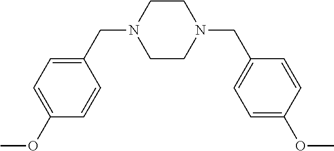 |
| 1293 | 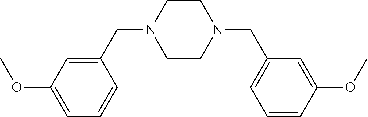 |
| 1294 | 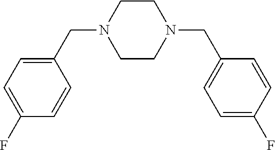 [SMILES: Fc1ccc(CN2CCN(Cc3ccc(F)cc3)CC2)cc1] |
| 1295 | 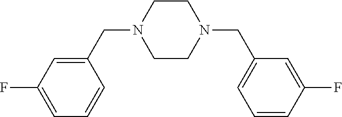 [SMILES: Fc1cccc(CN2CCN(Cc3cccc(F)c3)CC2)c1] |
| 1336 | 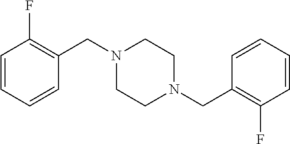 [SMILES: Fc1ccccc1CN1CCN(Cc2ccccc2F)CC1] |
| 1337 | 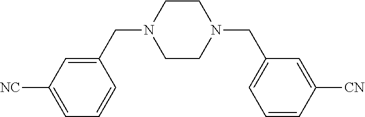 [SMILES: N#Cc1cccc(CN2CCN(Cc3cccc(C#N)c3)CC2)c1] |
| 1338 | 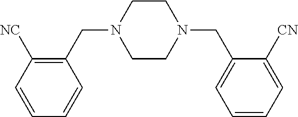 [SMILES: N#Cc1ccccc1CN1CCN(Cc2ccccc2C#N)CC1] |
| 1339 | 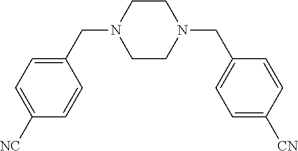 [SMILES: N#Cc1ccc(CN2CCN(Cc3ccc(C#N)cc3)CC2)cc1] |
| 1365 | 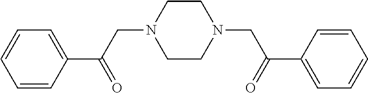 [SMILES: O=C(CN1CCN(CC(=O)c2ccccc2)CC1)c1ccccc1] |

### Example 6—Preparation of R20 Analogues of Class E

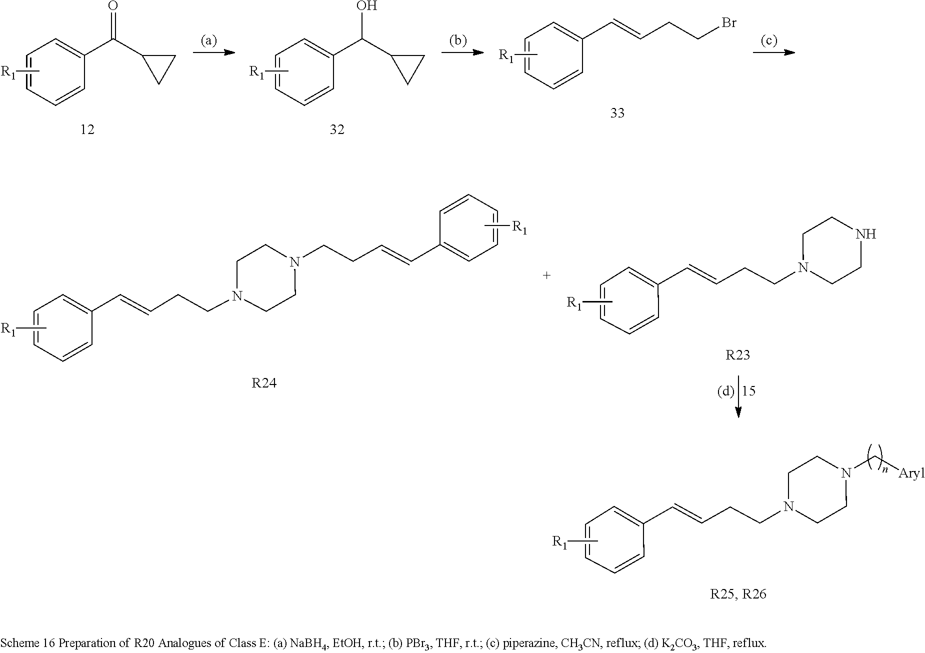

Compounds of R20 analogues of class E can be prepared by typical methods as illustrated in Scheme 16. R23 and R24 were prepared by the typical procedure as described above for intermediates 21. By the alkylation of R23, compounds of R20 analogues of class E: R25 and R26 were obtained.

General procedure for the preparation of R20 Analogues of Class E: A mixture of 33 (8.73 mmol), piperazine (3.76 g, 43.7 mmol) in 40 mL acetonitrile was stirred at reflux overnight. After cooling to r.t., the acetonitrile was removed by evaporation. Diluted with EtOAc, the organic layer was washed with H₂O, dried over Na₂SO₄, filtered and concentrated. The crude residue was purified by chromatography to give R20 Analogues of Class E: R23 and R24.

A mixture of R23 (1 mmol), halides 15 (3 mmol, 3 eq), and K₂CO₃ (10 mmol, 10 eq) in THF (25 mL) was stirred overnight at 67° C. After cooling to r.t., the reaction mixture was filtered. The filtrate was concentrated. The crude residue was purified by chromatography to give R20 Analogues of Class E: R25 and R26.

Characterization of R20 Analogues of Class E:

R23: ¹H NMR (500 MHz, CDCl₃) δ 7.34-7.28 (m, 2H), 6.98-7.02 (m, 2H), 6.40 (d, J=15.9 Hz, 1H), 6.12 (dt, J=15.8, 6.9 Hz, 1H), 3.64 (brs, 1H), 2.98 (t, J=5.0 Hz, 4H), 2.68-2.48 (m, 6H), 2.39-2.44 (m, 2H). HRMS (ESI) m/z Found: 235.16148 [M+H]⁺, Calcd: 235.16050.

R24: ¹H NMR (500 MHz, CDCl₃) δ 7.33-7.27 (m, 4H), 7.02-6.94 (m, 4H), 6.39 (d, J=15.9 Hz, 2H), 6.11 (dt, J=15.8, 6.9 Hz, 2H), 2.78-2.47 (m, 12H), 2.46-2.35 (m, 4H). HRMS (ESI) m/z Found: 383.23008 [M+H]⁺, Calcd: 383.22933.

R25: Syrup, 74.2% yield. ¹H NMR (500 MHz, CDCl₃) δ 7.61-7.52 (m, 3H), 7.52-7.40 (m, 2H), 7.28 (dd, J=8.7, 5.4 Hz, 2H), 6.97 (t, J=8.7 Hz, 2H), 6.37 (d, J=15.8 Hz, 1H), 6.15-6.05 (m, 1H), 4.77 (s, 2H), 2.95-2.36 (m, 12H).

R26: white solid, 61.9% yield. ¹H NMR (500 MHz, DMSO-d₆) δ 7.42-7.30 (m, 4H), 7.22-7.14 (m, 4H), 6.61 (d, J=15.9 Hz, 1H), 6.13-6.20 (m, 1H), 4.71 (s, 2H), 3.48-3.40 (m, 4H), 2.92-2.65 (m, 6H). HRMS (ESI) m/z Found: 343.1990 [M+H]⁺, Calcd: 343.1980.

TABLE 6
Structures of R20 Analogues of Class C.

| | |
|---|---|
| R23 | 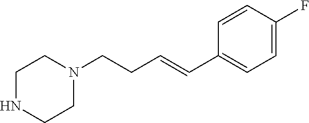 |
| R24 | 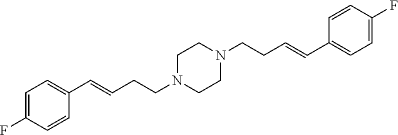 |
| R25 | 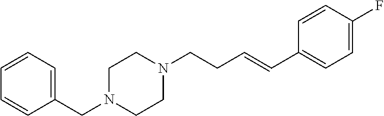 |
| R26 | 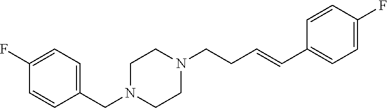 |

Compound V188 is known in the art. Its chemical structure is outlined below.

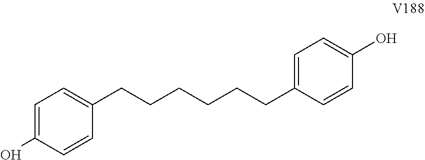 [SMILES: Oc1ccc(CCCCCCc2ccc(O)cc2)cc1]

TABLE 7
Additional compounds prepared in a subsequent part of the invention.

| ID | Structure |
|---|---|
| M62 | 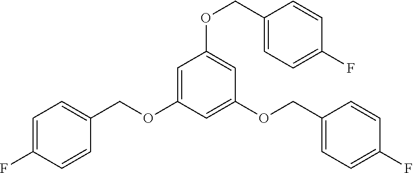 [SMILES: Fc1ccc(COc2cc(OCc3ccc(F)cc3)cc(OCc3ccc(F)cc3)c2)cc1] |
| M64 | 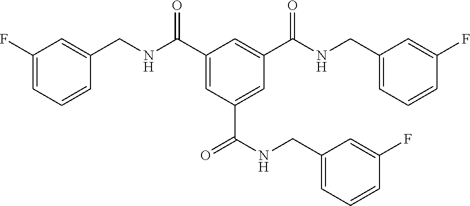 [SMILES: O=C(NCc1cccc(F)c1)c1cc(C(=O)NCc2cccc(F)c2)cc(C(=O)NCc2cccc(F)c2)c1] |
| M65 | 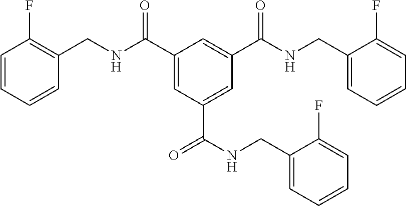 [SMILES: O=C(NCc1ccccc1F)c1cc(C(=O)NCc2ccccc2F)cc(C(=O)NCc2ccccc2F)c1] |
| M66 | 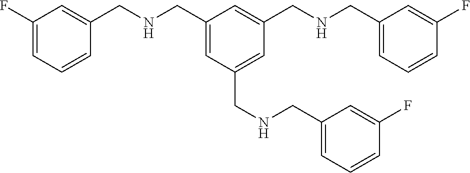 [SMILES: Fc1cccc(CNCc2cc(CNCc3cccc(F)c3)cc(CNCc3cccc(F)c3)c2)c1] |
| M67 | 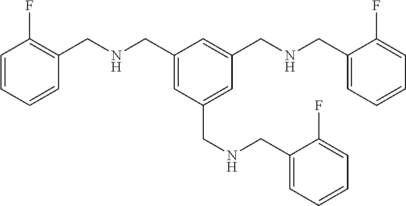 [SMILES: Fc1ccccc1CNCc1cc(CNCc2ccccc2F)cc(CNCc2ccccc2F)c1] |
| M74 | 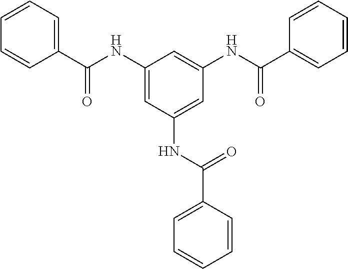 [SMILES: O=C(Nc1cc(NC(=O)c2ccccc2)cc(NC(=O)c2ccccc2)c1)c1ccccc1] |
| M75S | 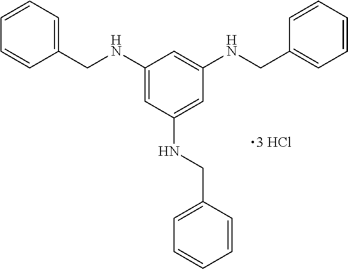 [SMILES: c1ccc(CNc2cc(NCc3ccccc3)cc(NCc3ccccc3)c2)cc1] |
| M77 | 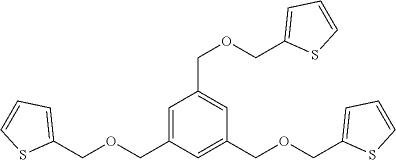 [SMILES: c1csc(COCc2cc(COCc3cccs3)cc(COCc3cccs3)c2)c1] |
| M79 | 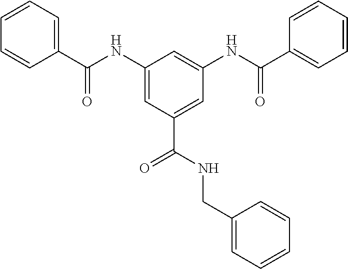 [SMILES: O=C(NCc1ccccc1)c1cc(NC(=O)c2ccccc2)cc(NC(=O)c2ccccc2)c1] |
| M80 | 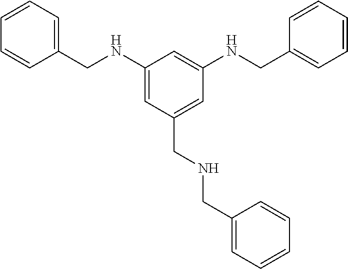 [SMILES: c1ccc(CNCc2cc(NCc3ccccc3)cc(NCc3ccccc3)c2)cc1] |
| M80S | 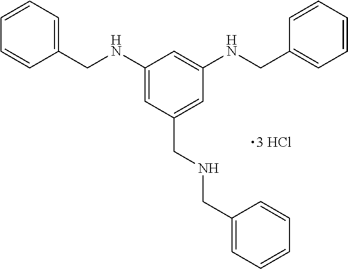 [SMILES: c1ccc(CNCc2cc(NCc3ccccc3)cc(NCc3ccccc3)c2)cc1] |
| M81 | 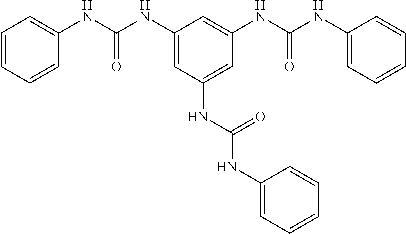 [SMILES: O=C(Nc1ccccc1)Nc1cc(NC(=O)Nc2ccccc2)cc(NC(=O)Nc2ccccc2)c1] |
| M82 | 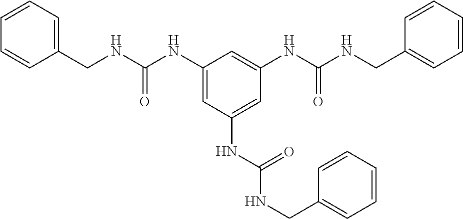 [SMILES: O=C(NCc1ccccc1)Nc1cc(NC(=O)NCc2ccccc2)cc(NC(=O)NCc2ccccc2)c1] |
| M83 | 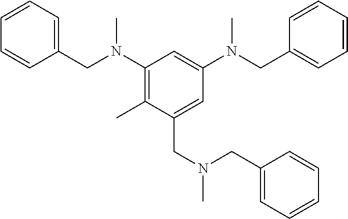 [SMILES: Cc1c(CN(C)Cc2ccccc2)cc(N(C)Cc2ccccc2)cc1N(C)Cc1ccccc1] |
| M84 | 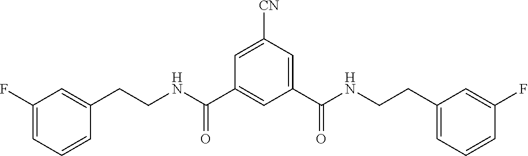 [SMILES: N#Cc1cc(C(=O)NCCc2cccc(F)c2)cc(C(=O)NCCc2cccc(F)c2)c1] |
| M85 | 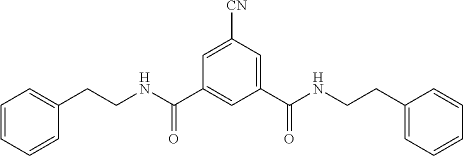 [SMILES: N#Cc1cc(C(=O)NCCc2ccccc2)cc(C(=O)NCCc2ccccc2)c1] |
| M86 | 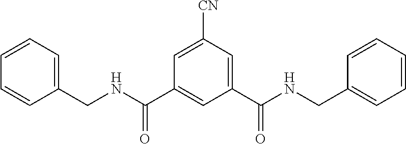 [SMILES: N#Cc1cc(C(=O)NCc2ccccc2)cc(C(=O)NCc2ccccc2)c1] |
| M87 | 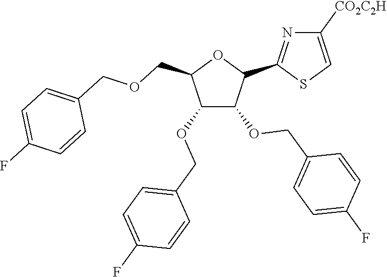 |
| M88 | 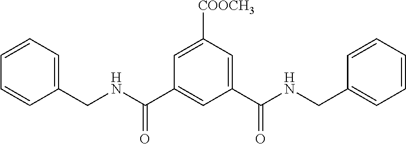 [SMILES: COC(=O)c1cc(C(=O)NCc2ccccc2)cc(C(=O)NCc2ccccc2)c1] |
| M89 | 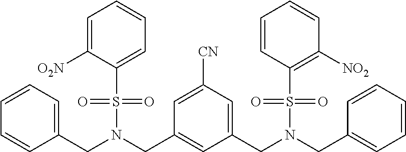 |
| M90 | 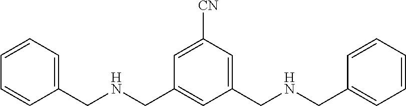 [SMILES: N#Cc1cc(CNCc2ccccc2)cc(CNCc2ccccc2)c1] |
| M91 | 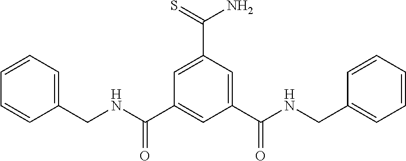 [SMILES: NC(=S)c1cc(C(=O)NCc2ccccc2)cc(C(=O)NCc2ccccc2)c1] |
| M92 | 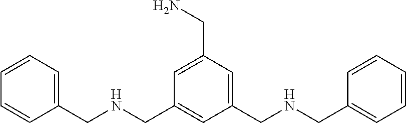 [SMILES: NCc1cc(CNCc2ccccc2)cc(CNCc2ccccc2)c1] |
| M93 | 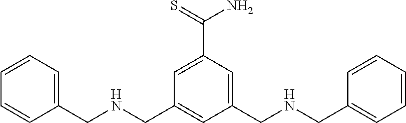 [SMILES: NC(=S)c1cc(CNCc2ccccc2)cc(CNCc2ccccc2)c1] |
| M94 | 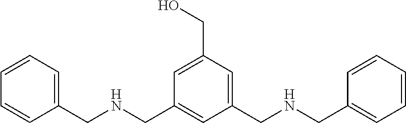 [SMILES: OCc1cc(CNCc2ccccc2)cc(CNCc2ccccc2)c1] |
| M95 | 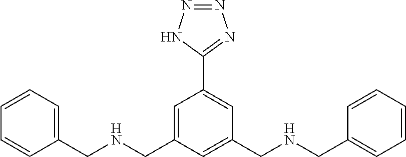 [SMILES: c1ccc(CNCc2cc(CNCc3ccccc3)cc(-c3nnn[nH]3)c2)cc1] |
| M96 | 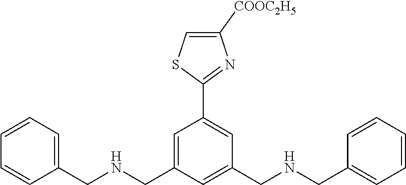 |

### Materials and Methods

Cell Lines and Cell Culture:

The human EOC cell lines used (TOV81D, TOV112D, OV90, TOV21G, OV866(2), TOV1369(R), OV1369(R2), TOV1946, OV1946, TOV2295(R), OV4485) were derived in our laboratory from patients' tumors (TOV) or ascites (OV) [7,10-12]. All EOC cell lines were maintained in a low oxygen condition of 7% O₂ and 5% CO₂ and grown in OSE medium (Wisent, Montreal, QC) supplemented with 10% FBS (Wisent), 0.5 μg/mL amphotericin B (Wisent) and 50 μg/mL gentamicin (Life Technologies Inc., Burlington, ON). The human retinal epithelial cell line ARPE-19 was purchased from American Type Culture Collection (ATCC, Manassas, Va.) and maintained in DMEM-F12 (Wisent) supplemented with 10% FBS (Wisent), 0.5 μg/mL amphotericin B (Wisent) and 50 μg/mL gentamicin (Life Technologies Inc.).

Small Interference RNA (siRNA) Treatment:

Suspensions of 10⁶ cells in 100 μL of nucleofector solution V (Lonza Group Ltd, Basel, Switzerland) were transfected by electroporation with 1.2 nmoles siRNA targeting Ran (J-010353-06, ON-TARGETplus, Dharmacon Thermo Fisher Scientific Inc., Waltham, Mass.). For each experiment, efficiency of Ran silencing was verified 48 hours after transfection by Western blotting. Scramble siRNA (D-001810-02, Dharmacon) was used as control in all the experiments.

Clonogenic Survival Assay to Measure Drug Sensitivity:

Clonogenic assays were performed as previously described [10,11]. Colonies were counted under a stereo microscope and reported as percent of control. IC₅₀ values were determined using Graph Pad Prism 5 software (GraphPad Software Inc., San Diego, Calif.). Each experiment was performed in duplicate and repeated three times. Sensitivity of the cell lines to small molecules inhibitors of Ran was assessed using a concentration range of 0-50 μM.

IncuCyte Cell Proliferation Phase-Contrast Imaging Assay:

Cells (2,000 cells/well) were plated in a 96-well plate. The next day, compounds were added at the indicated concentrations. Following treatment, cell confluence was imaged by phase contrast using the IncuCyte live cell monitoring system (Essen BioScience, Ann Arbor, Mich.). Frames were captured at 2-hour intervals using a 10× objective. For Ran knock down experiments, cells were seeded in a 96-well plate (4,000 cells/well) directly after transfection. Cell confluence monitoring started the next day as described above.

Protein Preparation and Western Blot Analysis:

Cells were lysed with RIPA buffer containing protease inhibitors. Whole cell lysates were run through a Bradford assay (Thermo Fisher Scientific) for protein quantification. Around 25-50 μg of proteins were separated onto 12.5% SDS-PAGE and transferred onto nitrocellulose membranes. The resultant blots were probed with Ran (1:10000, sc-271376 Santa Cruz Biotechnology, Dallas, Tex.), cleaved PARP (1:1000, #9541, Cell Signaling Technology Inc., Danvers, Mass.), GAPDH (1:2500, #2118, Cell Signaling Technology Inc.) or beta-actin (1:50000, ab6276, Abcam Inc., Toronto, ON, Canada) primary antibodies overnight at 4° C. then with peroxidase-conjugated secondary antibodies for 2 hours at room temperature. Proteins were detected using enhanced chemiluminescence (Thermo Fisher Scientific).

Apoptosis Analysis by Flow Cytometry:

Cells were transfected with siRan or siScr and seeded in 6-well plates. Ninety six hours after transfection, cells were collected and incubated 30 minutes at room temperature with BV421 Annexin V (563973, BD Biosciences, San Jose, Calif.) and 5 minutes at room temperature with DRAQ 7 (ab109202, Abcam Inc). A maximum of 30,000 events were counted per condition using the Fortessa flow cytometer (BD Biosciences, Mississauga, ON) and analyzed with the FlowJo software.

Analysis of Active Ran-GTP on Mitotic Cells:

Cells were grown in 150-mm petri dishes to approximately 70% confluency and treated with nocodazole (300 nM) overnight. After PBS wash (to remove dead cells), Petri dishes were vigorously shaken for 10 seconds and media containing cells in suspension were used for cell cycle analyses (to confirm the enrichment of mitotic cells) and for Ran activation assay. For cell cycle analysis by flow cytometry, cells were fixed for 24 hours in 70% ethanol and incubated for 30 minutes at room temperature with 100 μg/mL RNAse A and 25 μg/mL propidium iodide (PI).

Induction of Aneuploidy with Cytochalasin D:

Diploid ARPE-19 and TOV81D cells were treated with nocodazole (300 nM) overnight. After two washes with complete medium, cells were treated with cytochalasin D (2.5 μg/mL) for 6 hours then washed again twice and incubated with fresh media overnight. Cells were then transfected with siRan and cell proliferation was measured using the IncuCyte system. For these experiments, the induction of tetraploidy was verified by immunofluorescence. Treated cells were fixed, permeabilized and stained with alpha tubulin antibody conjugated with FITC (1:500, clone DM1A, Sigma-Aldrich Inc., St. Louis, Mo.) and DAPI. The number of binucleated cells were counted using a Zeiss microscope (Zeiss observer Z1).

Drugs:

Small molecules inhibitors of Ran were dissolved in 100% dimethyl sulfoxide (DMSO) and then further diluted in complete culture media for in vitro experiments. Drugs were added 24 hours after seeding.

Ran-GTP Immunofluorescence:

M36 and R28-treated and control TOV112D cells grown on coverslips were washed with 1×PBS, fixed in 4% paraformaldehyde and permeabilized with 0.25% Triton X-100 (Sigma-Aldrich Inc.). After blocking (4% BSA and 4% FBS in PBS), coverslips were incubated with the monoclonal anti-RanGTP antibody (26915, NewEastBioscences) diluted 1:100 in blocking buffer for 2 hours at room temperature. Subsequently, samples were incubated with Cy-5 secondary antibody (1:500, Life Technologies Inc.) for 1 hour and coverslips were mounted onto slides using Prolong® Gold anti-fade reagent with DAPI (Life Technologies Inc.). Samples were visualized under a Zeiss microscope (Zeiss observer Z1) with a 20× objective.

Surface Plasmon Resonance (SPR):

SPR experiments were carried out using the Biacore 3000 system. Recombinant Ran protein was purchased from Sigma-Aldrich Inc. (R3152). The running buffer contained PBS, pH7.4, 1 mM GDP, 2 mM MgCl₂ and 0.2% DMSO. The regeneration buffer contained 10 mM glycine (pH 2.5). Ran-GDP protein was immobilized onto a CM5 chip; samples of compounds in running buffer were injected at 30 μL/min for 10 minutes contact time followed by 5 minutes regeneration. Kd was calculated using the GraphPad Prism 5 software.

Ran Activation Assay:

Cells were seeded onto 6-well tissue culture plates in such a way that cell confluence reaches approximately 70% the day of experiment. The day of experiment cells were treated for 1 hour with the indicated compounds prior to protein extraction and quantification. Assays were performed using the Ran activation assay kit (Cell Biolabs). Briefly, 400 μg of lysates were incubated for one hour at 4° C. with agarose beads conjugated to RANBP1, which specifically binds Ran-GTP. Beads were pelleted, washed, and re-suspended in SDS-PAGE buffer, followed by immunoblotting with an anti-Ran antibody.

Pharmacokinetics and Tolerance Experiments in Mice:

For the pharmacokinetic studies, 6-week-old female CD1 mice (Charles River laboratories, Senneville, QC, Canada) received a single intravenous or intraperitoneal injection of M36 or QR20 (50 mg/kg), dissolved in DMSO 10%, Kolliphor® EL 10%, PEG-400 20% and PBS 60% (QR20 was also dissolved in DMSO 10%, PBS 90%). For each time point (15 minutes, 30 minutes, 60 minutes, 1 hour, 2 hours and 6 hours), 3 mice were sacrificed and blood was collected by cardiac puncture. Thereafter, the plasma level of each compound was measured by mass spectrometry.

For the tolerance test, M36 and QR20 compounds were dissolved in DMSO 10%, Kolliphor® EL 10%, PEG-400 20% and PBS 60% and injected intraperitoneally into 6-week-old female Nod Rag Gamma (NRG) mice (The Jackson laboratory, Bar Harbor, Me.) daily at 75 mg/kg. During this study, mice (n=3) were monitored for survival and weight loss/gain.

### Results

Impact of Aneuploidy on Ran Knockdown Sensitivity:

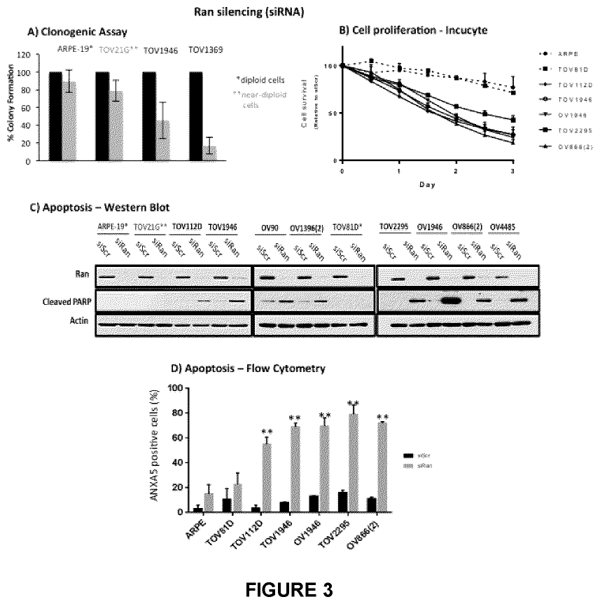

Before developing small molecules inhibitors of the GTPase Ran to target aneuploid cancer cells, we needed to test our hypothesis that these cells are more dependent on Ran activity than normal diploid cells. First we investigated the sensitivity of several EOC cell lines to Ran knockdown. Our results using siRNA against Ran and clonogenic assay show that the EOC cell lines TOV112D, TOV1369 and TOV1946, which have aberrant karyotypes, are more sensitive to Ran knockdown than normal diploid retina epithetlial cells (ARPE-19) and near-diploid TOV21G cells (FIG. 3A). Importantly, these aneuploid cell lines are categorized as resistant based on their sensitivity to carboplatin but appear to be sensitive to the loss of Ran, a finding that supports targeting Ran even in the context of platinum-resistant disease. These results were confirmed using a cell proliferation assay (assessed by live cell imaging using the IncuCyte system) and other aneuploid HGS cell lines (TOV2295(R), OV866(2) and OV1946), and a diploid EOC cell line (TOV81D) (FIG. 3B). Furthermore, apoptosis analysis of several EOC cell lines confirms that Ran knockdown induces PARP cleavage (Western blot, FIG. 3C) and increases the number of Annexin V positive cells (Flow cytometry, FIG. 3D) only in aneuploid HGS EOC cells.

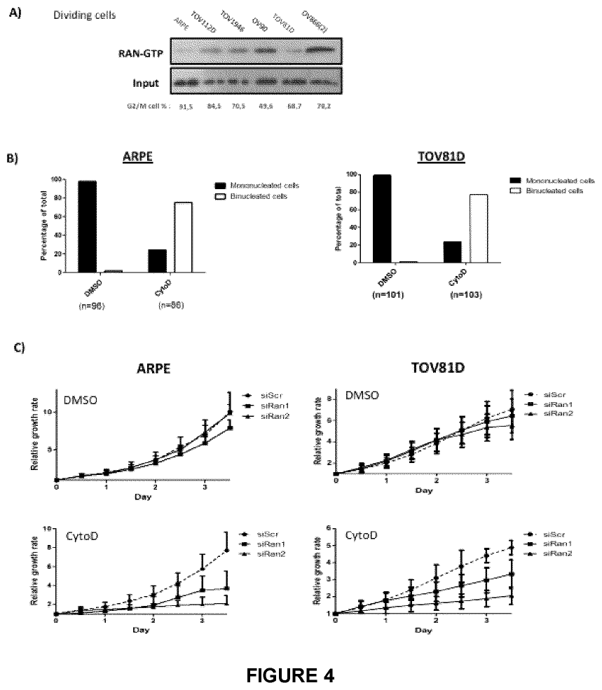

In line with our hypothesis that cells with aberrant chromosomal content need higher Ran activity during mitosis than diploid cells, we showed that aneuploid EOC cells that were synchronized in the G2/M phase of the cell cycle have higher levels of active Ran-GTP (assessed by an specific Ran-GTP antibody) than normal (ARPE-19) or tumoral (TOV81D) diploid cells that had as well been synchronized at this cell cycle phase (FIG. 4A). Importantly, induction of tetraploidy in these two diploid cell lines, by cytochalasin D treatment (an actin polymerization inhibitor that inhibit cytokinesis by cleavage failure at the end of mitosis [49]), sensitizes these cells to Ran knockdown (FIGS. 4B-C).

Screening of Selected NCI Compounds:

Having established that aneuploid HGS EOC cells are more sensitive to Ran knockdown than normal or tumoral diploid cells, we went further towards our goal to develop new small molecules inhibitors of Ran. This was performed using our extensive experience in drug design, chemical synthesis and in silico screening [50-55]. Although the crystal structure of Ran is available (PDB entry 1BBR, 3CH5), no chemical inhibitors of Ran have previously been reported. Because of the natural high affinity of GTP when binding to Ran, the GTP-pocket itself is widely considered difficult, if not impossible, to target by a small molecule approach. Therefore, the approach chosen was to target the GDP-bound form of Ran, with the hypothesis that this would lock the protein in an inactive state, thereby depleting the active Ran population. By visually inspecting Ran's molecular and structural surface, we selected a binding-site on the surface of Ran, which included the GDP-binding pocket and an allosteric sub-pocket, to apply a virtual screening using an in silico modeling approach developed by us.

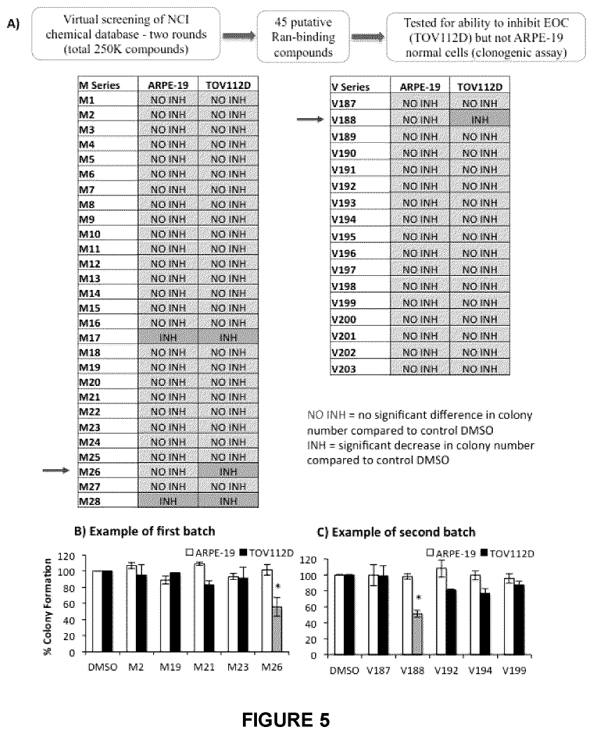

Based on this strategy, the NCI chemical database (total of 250,000 compounds) was virtually screened in two steps, 90 thousands compounds first then the remaining 160 thousands. Top-ranking compounds identified in this in silico screen went through a more in depth visual inspection for their chemical structures and binding modes. Following this selection, we obtained from the NCI 28 compounds from the first screening and 17 from the second as potential Ran inhibitors. Biological activity was assessed by clonogenic assays (at a single dose of 10 □M) using one aneuploid EOC cell line (TOV112D) and the normal ARPE-19 cells. Criterion for positive hit was that the compound did not inhibit the colony formation of the ARPE-19 cells but significantly inhibited the number of colonies for the TOV112D cells. Our results show that one compound from the first screening, M26, and one compound from the second screening, V188, specifically inhibited colony formation of EOC but not normal cells (FIGS. 5A-C).

Characterization and Validation of Lead Compounds:

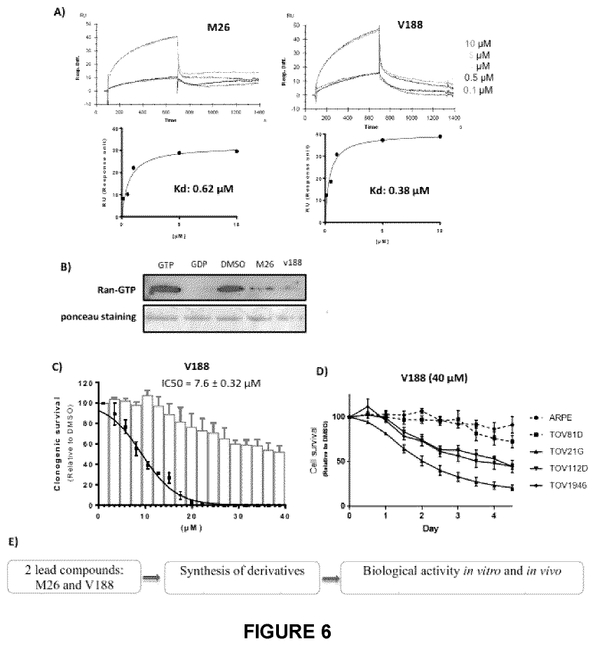

Since our screening was virtual using Ran crystal structure, it is therefore important to demonstrate that lead compounds are able to bind and inhibit Ran, and that this binding is specific for this particular GTPase. To address the binding issue, we determined the affinity of compounds M26 and V188 with Ran by Surface Plasmon Resonance (SPR) analysis using recombinant human Ran protein. Our results show a concentration-dependent binding of both compounds to the immobilized Ran protein (FIG. 6A) that reached saturation, which is indicative of specific binding. The Kd values obtained were 0.62 μM for M36 and 0.38 μM for V188 (FIG. 6A). To evaluate the inhibitory activity of these compounds, levels of active GTP-bound Ran were evaluated on lysates of EOC cells treated or not with M26 or V188 using Ran pull down assays. Our results showed a marked decrease in Ran-GTP levels in TOV112D cells incubated with these compounds compared to vehicle DMSO-treated cells (FIG. 6B). Further in vitro analyses were performed with the V188 compound. The IC₅₀ value for the TOV112D cell line was determined by clonogenic assay (7.6±0.32 μM), where only minimal effect was observed for the normal ARPE-19 cell line at high concentrations (FIG. 6C). Similar to Ran knockdown by siRNA (FIG. 3B), we showed that V188 inhibited cell proliferation of other aneuploid EOC cells but not that of TOV81D (diploid EOC) cell line (FIG. 6D). Therefore, compounds M36 and V188 were chosen as scaffolds for subsequent optimization through medicinal chemistry. All the chemical structures, the medicinal chemistry strategies for the compounds according to the invention as well as their synthesis schemes are described herein (FIG. 6E). Also presented herein is the part of the invention related to the in vitro and in vivo biological activity of the compounds according to the invention.

Optimization of M26 Compound:

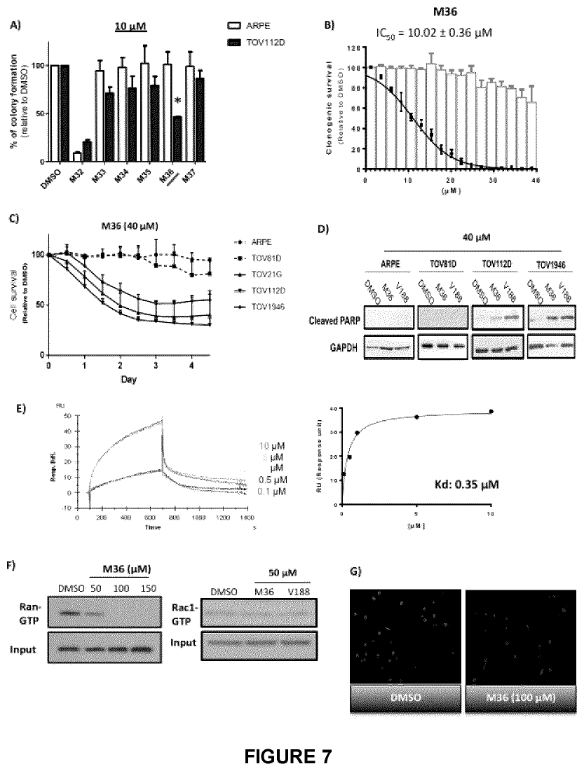

From the M26 structure, M32-M37 compounds were synthesized and tested in vitro. Our results showed that only compound M36 inhibited colony formation of TOV112D cells without affecting the ARPE-19 cells (FIG. 7A). Detailed study of the biological activity of this compound was performed. Its IC₅₀ value for the TOV112D cell line was determined by clonogenic assay (10.02±0.36 μM), where almost no effect was observed for the normal ARPE-19 cell line (FIG. 7B). We also showed that M36 inhibited cell proliferation of other aneuploid EOC cells but not that of TOV81D (diploid EOC) cell line (FIG. 7C). Furthermore, we showed that V188 and M36 induced cell apoptosis, as revealed by the presence of cleaved PARP on Western blot (FIG. 7D). Similar to M26 and V188, SPR analysis showed a concentration-dependent binding of M36 to the immobilized Ran protein (FIG. 7E) that reached saturation, with a Kd value of 0.35 μM. Using Ran pull down assays, we showed a concentration-dependent decrease in Ran-GTP levels in TOV112D cells when incubated with M36 compound (FIG. 7F). More importantly, when performing pull-down assays for another small GTPase Rac1, no decreased levels of Rac1-GTP was observed when incubated with V188 or M36 compounds (FIG. 7F), indicating specificity of these small molecules inhibitors towards Ran. In addition, immunofluorescence studies using an antibody specific to active Ran-GTP (see methods for details) showed no detectable levels of Ran-GTP in TOV112D cells treated with M36 as compared to intense staining of DMSO-treated cells (FIG. 7G). With those very optimistic results, other compounds analogs of M36 were synthesized.

Optimization of M36 Compound:

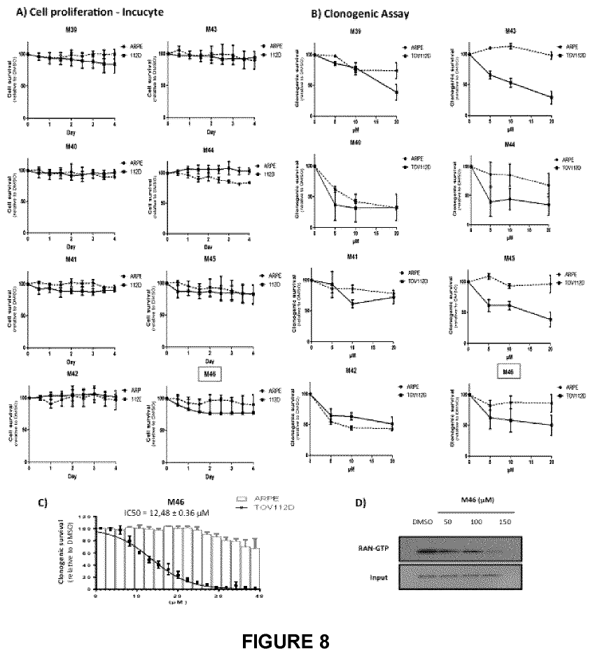

From the M36 structure, M39-M46 compounds were synthesized. Screening of these compounds was performed by two cell-based assays, proliferation (FIG. 8A) and clonogenic (FIG. 8B). Only compounds that showed inhibition of TOV112D cells and not that of normal ARPE-19 cells in both assays were considered for future analysis. From this series, only compound M46 showed some promising results. However, its IC₅₀ (12.48±0.36 μM) (FIG. 8C) and inhibition of Ran-GTP (FIG. 8D) was weaker than that of M36. Therefore, this compound was not further investigated.

Optimization of V188 Compound:

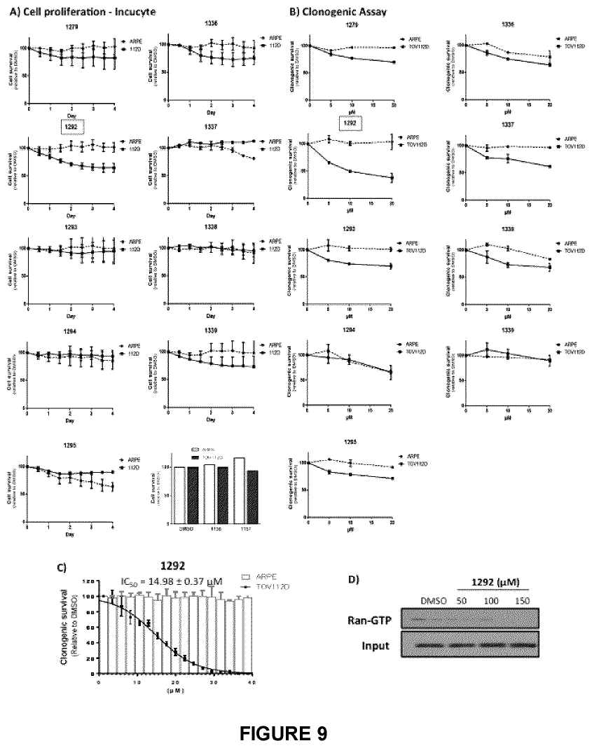

From the V188 structure, compounds 1156, 1157, 1279, 1292-1295 and 1336-1339 were synthesized and tested by clonogenic and cell proliferation assays (FIGS. 9A-B). Compound 1292 showed promising results. Although its IC50 was higher than that of the original V188 compound (14.98±0.37 μM), 1292 did not have any effect on ARPE-19 cells at any concentration tested (FIG. 9C), indicating a better therapeutic index for this compound. Using Ran pull down assays, we showed a concentration-dependent decrease in Ran-GTP levels in TOV112D cells when incubated with the 1292 compound (FIG. 9D).

Optimization of 1292 Compound:

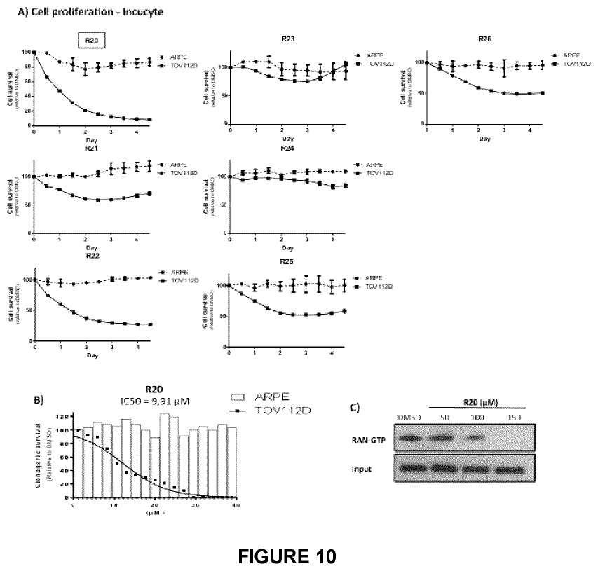

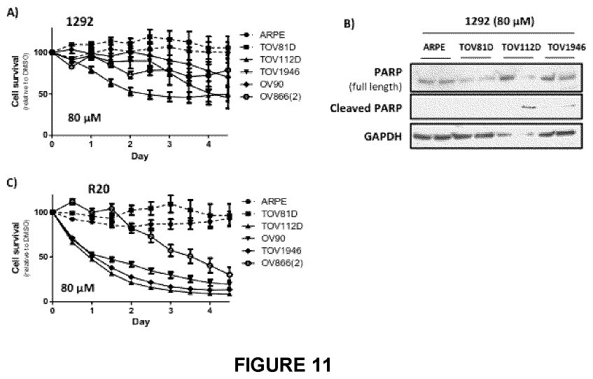

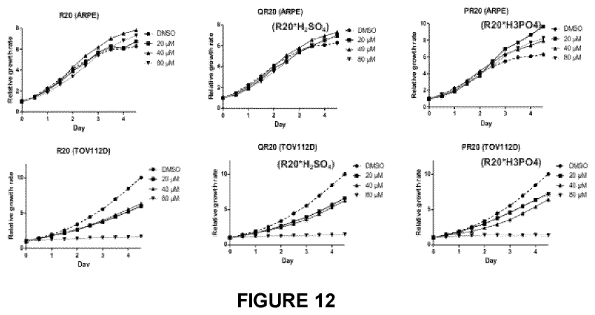

From the 1292 structure, R20-26 compounds were synthesized. Screening of these compounds was performed using a cell proliferation assay (FIG. 10A) and at least five compounds showed specific inhibition of TOV112D cells with no effect on the normal ARPE-19 cells. Compound R20 had greater efficacy in inhibiting cell proliferation and was selected for further analysis. The IC₅₀ value for the TOV112D cell line was determined by clonogenic assay (9.91±0.36 μM), where no effect was observed for the normal ARPE-19 cell line (FIG. 10B). Using Ran pull down assays, we showed a concentration-dependent decrease in Ran-GTP levels in TOV112D cells when incubated with R20 compound (FIG. 10C). We also showed that 1292 (FIG. 11A) and R20 (FIG. 11C) compounds inhibited cell proliferation of other aneuploid EOC cells but not that of TOV81D (diploid EOC) cell line, where R20 showed a greater efficacy and therapeutic index than the original 1292 compound. On Western blot, we confirmed the induction of apoptosis by the 1292 compound on TOV112D cells (FIG. 11B). The structure of R20 compound allows for further chemical modification and formulation to make it as a salt and therefore to be water soluble. Two different salt-formulations of R20 (*H₂SO₄, QR20; and *H₃PO₄, PR20) were tested on cell proliferation assays using different concentrations. Our results showed that both R20 salts have exactly the same activity to inhibit TOV112D cell proliferation without affecting the normal ARPE-19 cells (FIG. 12).

Pharmacokinetics and Tolerance Studies of Compounds M36 and QR20:

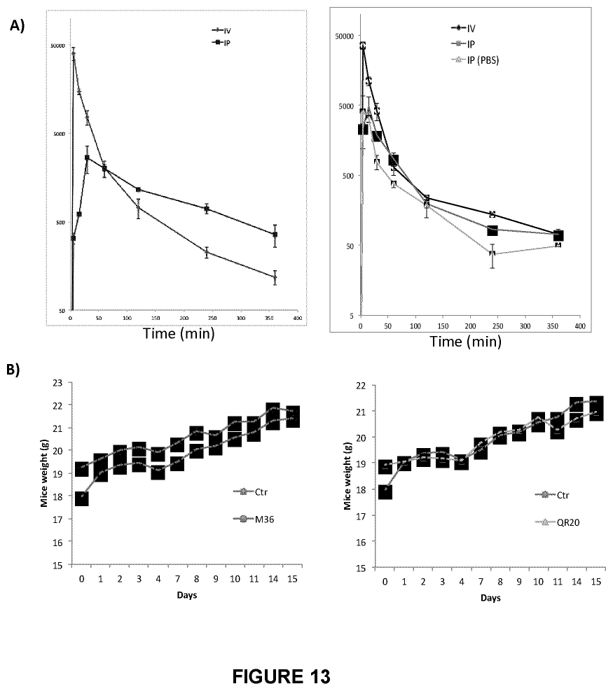

The promising in vitro results of R20 and M36 compounds lead us to initiate in vivo analysis of these small molecules inhibitors of Ran. For the R20 compound, the QR20 salt was selected and i.p. injected either in PBS or same vehicle as compound M36. Pharmacokinetic results showed that in comparison to intravenous injections, compound M36 is less absorbed than compound QR20, but persists longer in the circulation at the 50 mg/kg concentration used (FIG. 13A). Based on the area under the curve (AUC) values, both compounds have good pharmacokinetics profiles. Furthermore, we show that these two compounds are well tolerated, since no weight loss was observed after daily intraperitoneal injections (75 mg/kg) for 15 days (FIG. 13B).

Further Optimization of the M36 Compound:

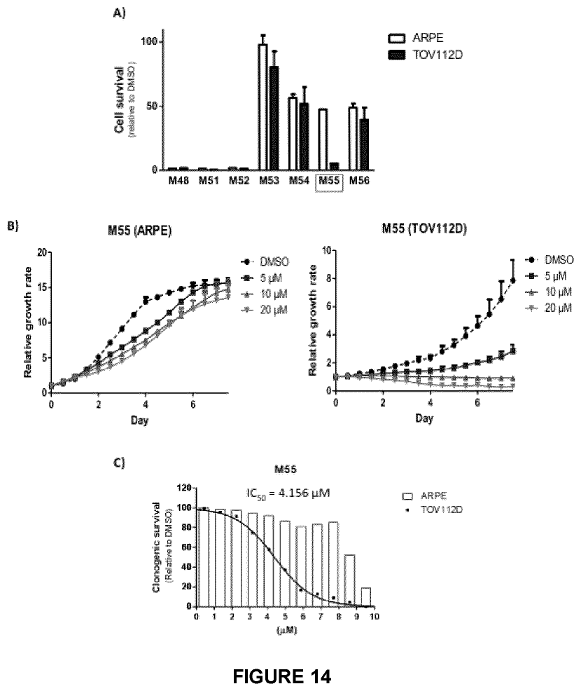

In parallel other compounds analogs of M36 and R20 were synthetized in order to improve their efficacy (i.e., to obtain lower IC₅₀ values). In the case of M36, compounds M48 and M51-M56 were synthesized. Screening of these compounds was performed by cell proliferation assay at concentrations of 40 μM using normal ARPE-19 and EOC TOV112D cells. Our results showed that compound M55 had a discriminative effect between normal ARPE-19 and aneuploid TOV112D cells (FIG. 14A). Detailed cell proliferation assays using different concentrations of compound M55 showed its efficacy to specifically inhibit cell growth of the TOV112D cells even at a low concentration of 5 μM (FIG. 14B). Further characterization by clonogenic assay was then performed, and the IC₅₀ value for the TOV112D cell line was calculated as 4.16 μM (FIG. 14C), which is around two times lower than that of compound M36 (10.02±0.36 μM) (FIG. 7B).

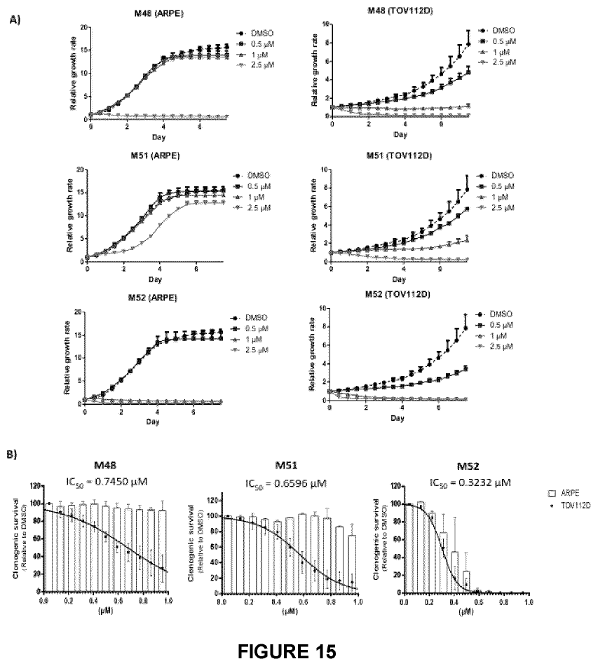

Since compounds M48, M51 and M52 completely inhibited cell growth of both EOC TOV112D and normal ARPE-19 (FIG. 14A), we decided to investigate whether a therapeutic index could be observed at lower concentrations of these compounds. FIG. 15 shows that this is the case for compound M51 that did not affect normal cells at concentration range from 0.5 to 2.5 μM but inhibited TOV112D cell growth at 1.0 and 2.5 μM. Further characterization by clonogenic assay showed that compound M51 is more effective than M36, since its IC₅₀ value (around 0.66 μM) for the TOV112D cell line (FIG. 15D) was 15 times lower than that of the M36. Although compounds M48 and M52 have also low IC₅₀ values, they also affected normal ARPE-19 cells at same low concentration range (FIG. 15).

Therefore, compound M51 is the first analog with efficacy lower than the micromolar range that presents a therapeutic window. These findings are encouraging and future experiments will be conducted to characterize the specificity, PK and the in vivo efficacy of this compound.

Optimization of R20 Compound:

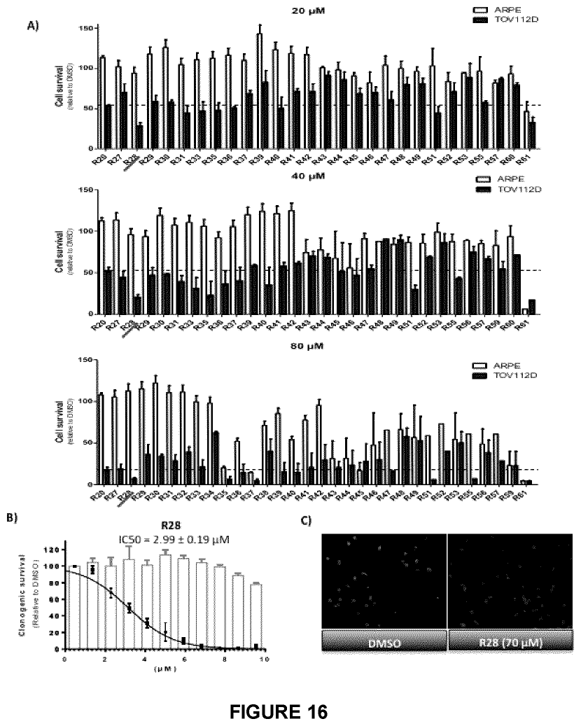

To better improve the efficacy of compound R20, other analogs were then synthesized: R27-49, R51-R53, R55-R57, R59 and R61 were produced. Screening of these compounds was performed by cell proliferation assay at concentrations of 20, 40 and 80 μM using normal ARPE-19 and EOC TOV112D cells. Our results showed that compound R28 was more efficient than compound R20 to inhibit proliferation of TOV112D cells without affecting normal ARPE-19 cells (FIG. 16A). Further characterization by clonogenic assay confirmed that compound R28 is more effective than compound R20, since its IC₅₀ value (2.99±0.19 μM) for the TOV112D cell line (FIG. 16B) was three times lower than that of the R20 (9.91±0.36 μM) (FIG. 10B). Furthermore, immunofluorescence studies using the anti-Ran-GTP antibody showed very low levels of active Ran-GTP in TOV112D cells treated with R28 as compared to intense staining of DMSO-treated cells (FIG. 16C), indicating its efficacy in inhibiting Ran. These are promising results and we plan to further characterize the binding properties of this compound and to perform PK and in vivo studies using the R28 compound. In addition, we also identified a second promising compound among these R20 Analogs, i.e. compound R51 (FIG. 16A), and further analyses will be conducted.

Effect of Selected Small Molecules Inhibitors of Ran on Other Cancer Models:

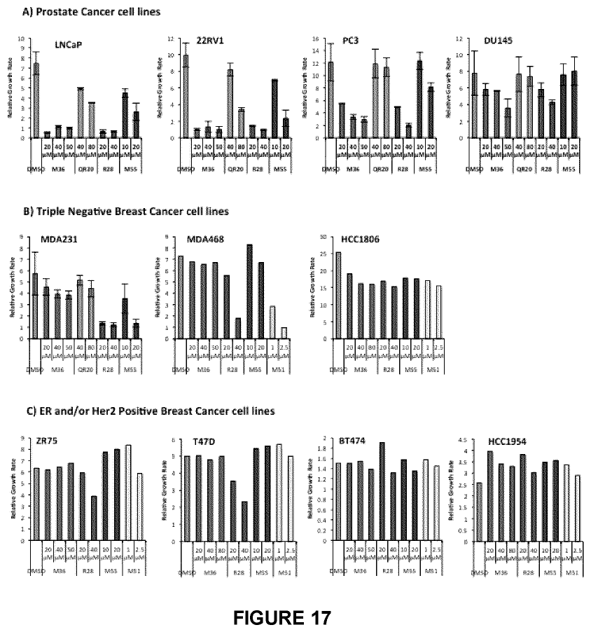

Although our work is focused on ovarian cancer, we wanted to verify whether the strategy of using small inhibitors of the GTPase Ran would be effective in other cancer types. Currently we have tested compounds M36, QR20, R28, M55 and M51 in several prostate and breast cancer cell lines (FIG. 17). Our results show that compound R28 (green bars) is effective in both cancer types, however, some variable sensitivity can be seen in the different cell lines studied. Compound M36 (orange bars) was also effective in prostate cancer cell lines but not in breast cancer, and compound M51 (cyan bars) was effective in one triple negative breast cancer cell line. Those results are encouraging, and indicate that some of our small molecules inhibitors of Ran could be exploited in the treatment of other cancer types.

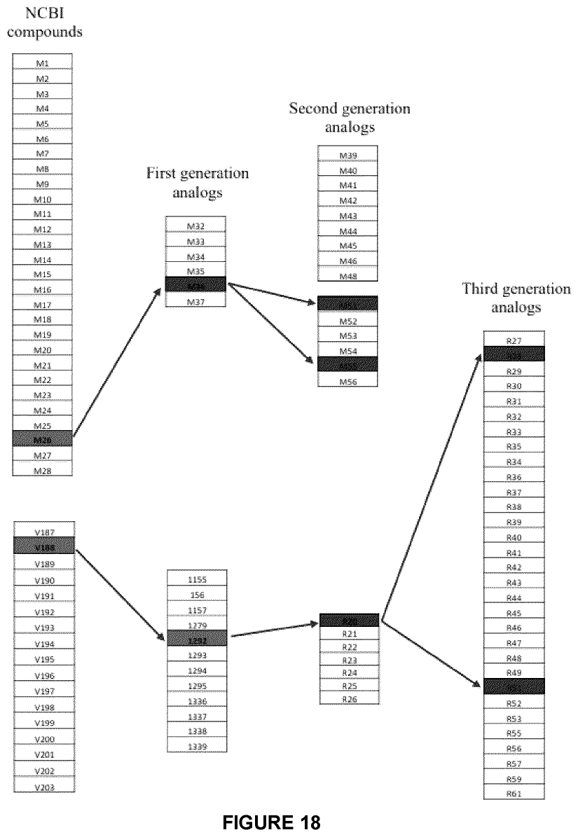

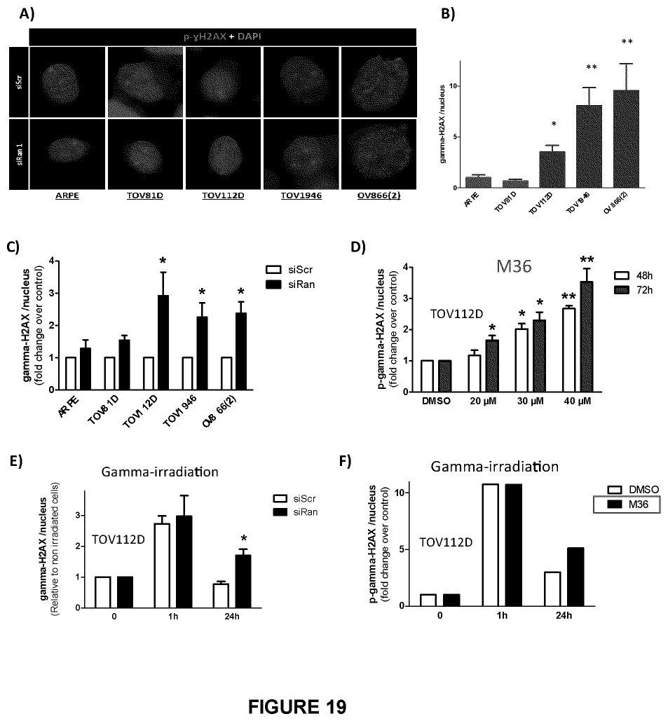

FIG. 18 relates to a first part of the invention and shows a summary of the compounds tested and synthetized. Compounds with promising biological activities are highlighted. In a subsequent part of the invention, additional compounds were synthesized and tested. These compounds are depicted in Table 7, and the biological results obtained are outlined in FIGS. 19-22.

Role of Ran GTPase in DNA Damage:

In a first part of the present invention and which is described herein, the inventors showed that downregulation of Ran GTPase by RNA interference (siRNA) or inhibition by small molecules induces selective cell death in aneuploid cancer cells without affecting normal diploid cells (FIGS. 3-4).

One of the characteristics differentiating aneuploid from diploid cells is the imbalance between sources of DNA damage and systems that control genome integrity. Aneuploid cells often displayed defects in proteins involved in DNA repair and/or DNA damage control (i.e., TP53 is mutated in 90% of HGSC cases) [63]. Indeed, our results showed enhanced phospho-gamma-H2AX (p-γH2AX) foci number (marker of DNA double-strand breaks) in our EOC aneuploid cells when compared to diploid cells (FIGS. 19A-B). These results indicate that the presence of spontaneous DNA damage is one of the characteristics of aneuploid cells. We then postulated that Ran might be involved in the DNA damage response (DDR) process, making aneuploid cancer cells dependent on Ran activity. To investigate if any association exists between Ran loss and DNA damage accumulation, cells were transfected with siRan before p-γH2AX foci analysis. We showed that, in contrast to diploid cells, Ran knockdown (KD) induced an accumulation of p-γH2AX foci in aneuploid cells (FIG. 19C). To confirm that Ran is involved in DNA damage repair, DNA damage was induced by gamma-irradiation and the clearance of p-γH2AX foci was monitored in TOV112D cells. As expected, an increased number of p-γH2AX foci was observed 1 hour after radiation exposure followed by a rapid clearance. However, p-γH2AX persisted longer when Ran was knocked-down (FIG. 19E).

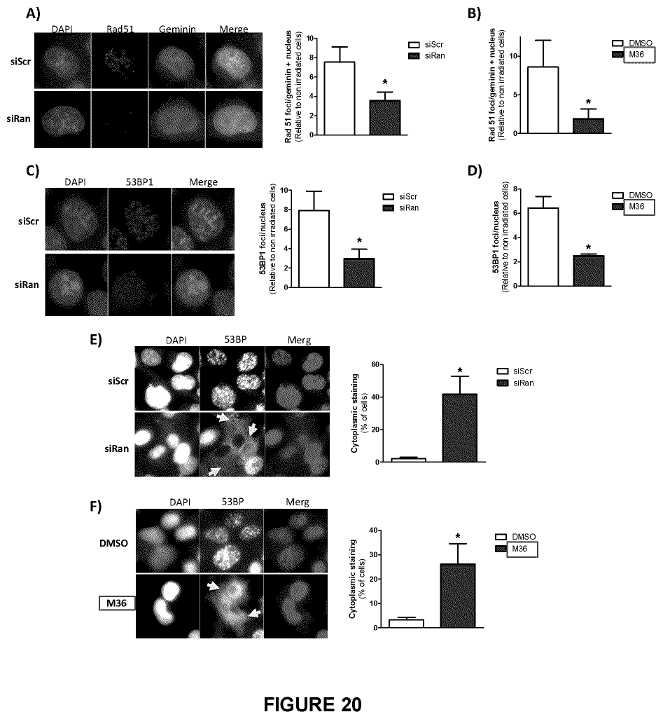

We then analyzed the functionality of two DNA double-strand break repair pathways, i.e. the homologous recombination (HR) and the non-homologous end joining (NHEJ), by quantifying Rad51 and 53BP1 foci, respectively. Our results showed decreased foci numbers for both markers when Ran was knocked down (FIGS. 20A and 20C). Furthermore, unlike the TOV112D control siScr cells, we observed diffuse cytoplasmic 53BP1 labeling in Ran KD cells (FIG. 20E). This is in agreement with a study showing that Ran is involved in the import of this protein from the cytoplasm to the nucleus through its partner importinβ [64].

Effects of Compound M36 on DNA Damage Repair:

To provide further evidence that our new small-molecule inhibitors of Ran are specific for this GTPase, we investigated the effect of our M36 compound on DNA damage and repair. Our results show that M36 recapitulates all the results obtained with Ran siRNA, i.e. it increases DNA damage (FIG. 19D), delays DNA double-strand breaks repair (FIG. 19F), by inhibiting the HR and NHEJ pathways (FIGS. 20B and 20D), and interferes with the nuclear localization of 53BP1 (FIG. 20F). These results not only indicate that our compounds targets Ran, but also imply that they might be used in combination with DNA damaging agents such as carboplatin but also with inhibitors of poly ADP ribose polymerase (PARP), such as Olaparib. The FDA has recently approved the use of PARP inhibitors for ovarian and prostate cancers, based on their synthetic lethal approach when HR is deficient. Therefore, HR inhibition by Ran inhibitors might induce synthetic lethality with PARPi as well.

Further Specificity Studies of Compound M36:

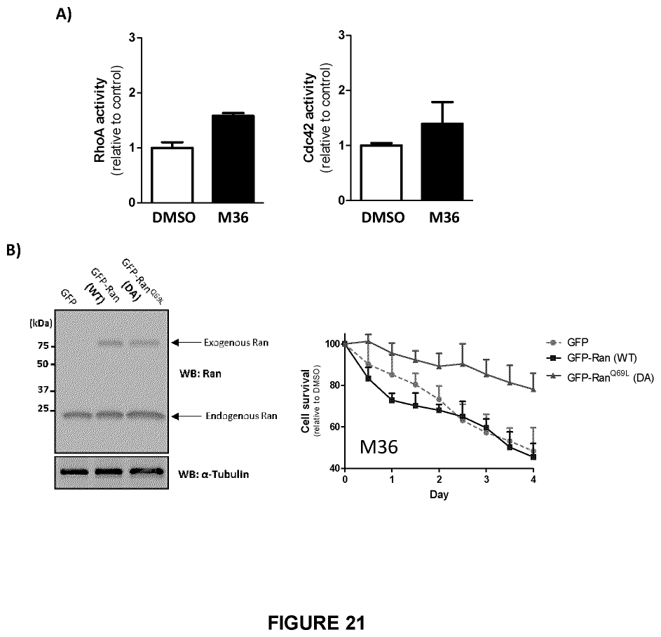

In a first part of the present invention and which is described herein, the inventors showed that compound M36 inhibits the activation of Ran GTPase, but not that of Rac-1 (FIG. 7), another small GTPase, demonstrating the specificity of our compounds. In a subsequent part of the invention also described herein, the inventors have extended their findings, showing that M36 does not inhibit the activation of RhoA or Cdc42 (FIG. 21A), two other small GTPases.

We also performed experiments to confirm our in silico screening model strategy, which predicted that our small-molecule inhibitors of Ran would bind to this GTPase on its GDP form. TOV112D cells were transfected with Ran wild type (WT) or with a dominant-active (DA) mutant, which maintains Ran in its GTP active conformation. Cells were then treated with compound M36 and cell survival was evaluated. Our results showed that the inhibition of cell proliferation induced by compound M36 was attenuated when DA Ran was overexpressed (FIG. 21B), suggesting that our compounds do not interact with Ran in its GTP form, confirming our in silico modeling strategy.

Biological Activity of Subsequently Synthesized Compounds According to the Invention:

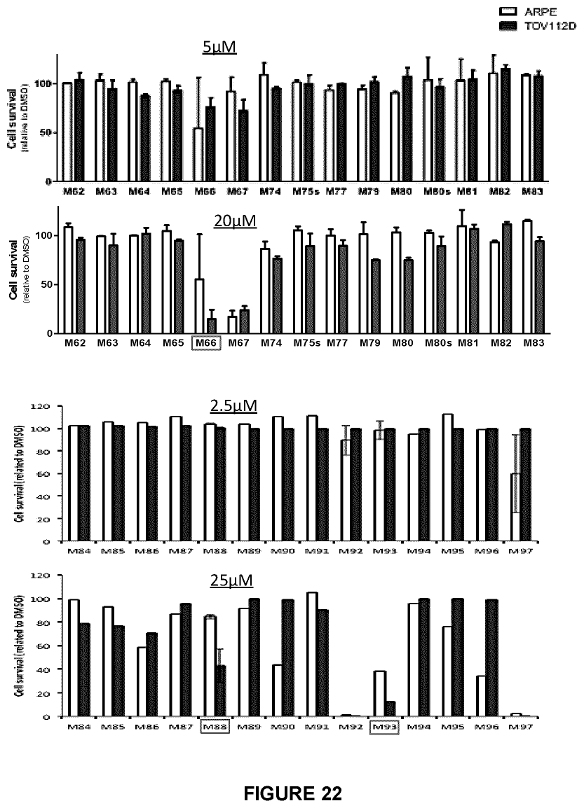

Compounds were synthesized and tested in vitro using diploid ARPE normal cells and aneuploid ovarian cancer cell line TOV112D. FIG. 22 shows that compound M88, and to a lesser extent compounds M66 and M93, show selective inhibition of cell proliferation of TOV112D but not ARPE.

The scope of the claims should not be limited by the preferred embodiments set forth in the examples, but should be given the broadest interpretation consistent with the description as a whole.

The present description refers to a number of documents, the content of which is herein incorporated by reference in their entirety.

### REFERENCES

1. Siegel R L, Miller K D, Jemal A: Cancer statistics, 2015. CA Cancer J. Clin. 65:5-29, 2015.
2. R. R. B, M. M, M. R: Principles and Practice of Gynecologic Oncology (5th ed.), Wolters Kluwer, 2009.
3. McGuire W P: Maintenance therapy for ovarian cancer: of Helsinki and Hippocrates. J. Clin. Oncol. 27:4633-4, 2009.
4. Coleman M P, Forman D, Bryant H, et al.: Cancer survival in Australia, Canada, Denmark, Norway, Sweden, and the UK, 1995-2007 (the International Cancer Benchmarking Partnership): an analysis of population-based cancer registry data. Lancet 377:127-38, 2011.
5. Bashashati A, Ha G, Tone A, et al: Distinct evolutionary trajectories of primary high-grade serous ovarian cancers revealed through spatial mutational profiling. J. Pathol. 231:21-34, 2013.
6. Network TCGAR: Integrated genomic analyses of ovarian carcinoma. Nature 474:609-15, 2011.
7. Ouellet V, Zietarska M, Portelance L, et al.: Characterization of three new serous epithelial ovarian cancer cell lines. BMC Cancer 8:152, 2008.
8. Press J Z, De Luca A, Boyd N, et al.: Ovarian carcinomas with genetic and epigenetic BRCA1 loss have distinct molecular abnormalities. BMC Cancer 8:17, 2008.
9. Stewart J M, Shaw P A, Gedye C, et al.: Phenotypic heterogeneity and instability of human ovarian tumor-initiating cells. Proc. Natl. Acad. Sci. U.S.A. 108:6468-73, 2011.
10. Fleury H, Communal L, Carmona E, et al.: Novel high-grade serous epithelial ovarian cancer cell lines that reflect the molecular diversity of both the sporadic and hereditary disease. Genes Cancer 6:378-98, 2015.
11. Letourneau I J, Quinn M C, Wang L L, et al.: Derivation and characterization of matched cell lines from primary and recurrent serous ovarian cancer. BMC Cancer 12:379, 2012.
12. Provencher D M, Lounis H, Champoux L, et al.: Characterization of four novel epithelial ovarian cancer cell lines. In Vitro Cell Dev. Biol. Anim. 36:357-61, 2000.
13. Le Page C, Ouellet V, Quinn M C, et al.: BTF4/BTNA3.2 and GCS as candidate mRNA prognostic markers in epithelial ovarian cancer. Cancer Epidemiol. Biomarkers Prev. 17:913-20, 2008.
14. Ouellet V, Ling T H, Normandin K, et al.: Immunohistochemical profiling of benign, low malignant potential and low grade serous epithelial ovarian tumors. BMC Cancer 8:346, 2008.
15. Lafontaine J, Rodier F, Ouellet V, et al.: Necdin, a p53-target gene, is an inhibitor of p53-mediated growth arrest. PLoS One 7:e31916, 2012.
16. Le Page C, Puiffe M L, Meunier L, et al.: BMP-2 signaling in ovarian cancer and its association with poor prognosis. J. Ovarian Res. 2:4, 2009.
17. Madore J, Ren F, Filali-Mouhim A, et al.: Characterization of the molecular differences between ovarian endometrioid carcinoma and ovarian serous carcinoma. J. Pathol. 220:392-400, 2010.
18. Normandin K, Peant B, Le Page C, et al.: Protease inhibitor SERPINA1 expression in epithelial ovarian cancer. Clin. Exp. Metastasis 27:55-69, 2010.
19. Abd-Rabbo D, Abaji C, Cardin G B, et al.: Allelic transcripts dosage effect in morphologically normal ovarian cells from heterozygous carriers of a BRCA1/2 French Canadian founder mutation. Cancer Prev. Res. (Phila) 5:765-77, 2012.
20. Cáceres-Gorriti K Y, Carmona E, Barrès V, et al.: RAN nucleo-cytoplasmic transport and mitotic spindle assembly partners XPO7 and TPX2 are associated with patient survival and recurrence in serous epithelial ovarian cancer. PLoS One 9:e91000, 2014.
21. Wojnarowicz P M, Oros K K, Quinn M C, et al.: The genomic landscape of TP53 and p53 annotated high grade ovarian serous carcinomas from a defined founder population associated with patient outcome. PLoS One 7:e45484, 2012.
22. Wojnarowicz P M, Provencher D M, Mes-Masson A M, et al.: Chromosome 17q25 genes, RHBDF2 and CYGB, in ovarian cancer. Int. J. Oncol. 40:1865-80, 2012.
23. Wojnarowicz P M, Gambaro K, Leclerc-Désualniers K, et al.: Overexpression of the CCL2 chemokine in an epithelial ovarian cancer cell line results in latency of in vivo tumourigenicity. Oncogenesis 1:e27, 2012.
24. Quinn M C, Wojnarowicz P M, Pickett A, et al.: FKBP10/FKBP65 expression in high-grade ovarian serous carcinoma and its association with patient outcome. Int. J. Oncol. 42:912-20, 2013.
25. Barres V, Ouellet V, Lafontaine J, et al.: An essential role for Ran GTPase in epithelial ovarian cancer cell survival. Mol. Cancer 9:272, 2010.
26. Lafontaine J, Tchakarska G, Rodier F, et al.: Necdin modulates proliferative cell survival of human cells in response to radiation-induced genotoxic stress. BMC Cancer 12:234, 2012.
27. Ouellet V, Guyot M C, Le Page C, et al.: Tissue array analysis of expression microarray candidates identifies markers associated with tumor grade and outcome in serous epithelial ovarian cancer. Int. J. Cancer 119:599-607, 2006.
28. Ouellet V, Provencher D M, Maugard C M, et al.: Discrimination between serous low malignant potential and invasive epithelial ovarian tumors using molecular profiling. Oncogene 24:4672-87, 2005.
29. Azuma K, Sasada T, Takedatsu H, et al.: Ran, a small GTPase gene, encodes cytotoxic T lymphocyte (CTL) epitopes capable of inducing HLA-A33-restricted and tumor-reactive CTLs in cancer patients. Clin. Cancer Res. 10:6695-702, 2004.
30. Li H, Ren C P, Tan X J, et al: Identification of genes related to nasopharyngeal carcinoma with the help of pathway-based networks. Acta Biochim. Biophys Sin (Shanghai) 38:900-10, 2006.
31. Xia F, Lee C W, Altieri D C: Tumor cell dependence on Ran-GTP-directed mitosis. Cancer Res. 68:1826-33, 2008.
32. Woo I S, Jang H S, Eun S Y, et al.: Ran suppresses paclitaxel-induced apoptosis in human glioblastoma cells. Apoptosis 13:1223-31, 2008.
33. Fan H, Lu Y, Qin H, et al.: High Ran level is correlated with poor prognosis in patients with colorectal cancer. Int. J. Clin. Oncol., 2012.
34. Sorokin A V, Kim E R, Ovchinnikov L P: Nucleocytoplasmic transport of proteins. Biochemistry (Mosc.) 72:1439-57, 2007.
35. Stewart M: Molecular mechanism of the nuclear protein import cycle. Nat. Rev. Mol. Cell Biol. 8:195-208, 2007.
36. Clarke P R, Zhang C: Spatial and temporal coordination of mitosis by Ran GTPase. Nat. Rev. Mol. Cell Biol. 9:464-77, 2008.
37. Ohtsubo M, Okazaki H, Nishimoto T: The RCC1 protein, a regulator for the onset of chromosome condensation locates in the nucleus and binds to DNA. J. Cell Biol. 109:1389-97, 1989.
38. Bischoff F R, Krebber H, Smirnova E, et al.: Co-activation of RanGTPase and inhibition of GTP dissociation by Ran-GTP binding protein RanBP1. EMBO J. 14:705-15, 1995.
39. Matunis M J, Coutavas E, Blobel G: A novel ubiquitin-like modification modulates the partitioning of the Ran-GTPase-activating protein RanGAP1 between the cytosol and the nuclear pore complex. J. Cell Biol. 135:1457-70, 1996.
40. Mahajan R, Delphin C, Guan T, et al.: A small ubiquitin-related polypeptide involved in targeting RanGAP1 to nuclear pore complex protein RanBP2. Cell 88:97-107, 1997.
41. Kalab P, Weis K, Heald R: Visualization of a Ran-GTP gradient in interphase and mitotic Xenopus egg extracts. Science 295:2452-6, 2002.
42. Deng L, Lu Y, Zhao X, et al.: Ran GTPase protein promotes human pancreatic cancer proliferation by deregulating the expression of Survivin and cell cycle proteins. Biochem. Biophys. Res. Commun. 440:322-9, 2013.
43. Morgan-Lappe S E, Tucker L A, Huang X, et al.: Identification of Ras-related nuclear protein, targeting protein for xenopus kinesin-like protein 2, and stearoyl-CoA desaturase 1 as promising cancer targets from an RNAi-based screen. Cancer Res. 67:4390-8, 2007.
44. Xia F, Canovas P M, Guadagno T M, et al.: A survivin-ran complex regulates spindle formation in tumor cells. Mol. Cell Biol. 28:5299-311, 2008.
45. Cheung H W, Cowley G S, Weir B A, et al.: Systematic investigation of genetic vulnerabilities across cancer cell lines reveals lineage-specific dependencies in ovarian cancer. Proc. Natl. Acad. Sci. U.S.A. 108:12372-7, 2011.
46. Marcotte R, Brown K R, Suarez F, et al.: Essential gene profiles in breast, pancreatic, and ovarian cancer cells. Cancer Discov. 2:172-89, 2012.
47. Hasegawa K, Ryu S J, Kalab P: Chromosomal gain promotes formation of a steep RanGTP gradient that drives mitosis in aneuploid cells. J. Cell Biol. 200:151-61, 2013.
48. Uetake Y, Sluder G: Prolonged prometaphase blocks daughter cell proliferation despite normal completion of mitosis. Curr. Biol. 20:1666-71, 2010.
49. Uetake Y, Sluder G: Cell cycle progression after cleavage failure: mammalian somatic cells do not possess a “tetraploidy checkpoint”. J. Cell Biol. 165:609-15, 2004.
50. Wu J H, Miao W, Hu L G, et al.: Identification and characterization of novel Nrf2 inducers designed to target the intervening region of Keap1. Chem. Biol. Drug Des. 75:475-80, 2010.
51. Zhou J, Geng G, Batist G, et al.: Syntheses and potential anti-prostate cancer activities of ionone-based chalcones. Bioorg. Med Chem. Lett. 19:1183-6, 2009.
52. Zhou J, Geng G, Shi Q, et al.: Design and synthesis of androgen receptor antagonists with bulky side chains for overcoming antiandrogen resistance. J. Med. Chem. 52:5546-50, 2009.
53. Zhou J, Liu B, Geng G, et al.: Study of the impact of the T877A mutation on ligand-induced helix-12 positioning of the androgen receptor resulted in design and synthesis of novel antiandrogens. Proteins 78:623-37, 2010.
54. Liu B, Geng G, Lin R, et al.: Learning from estrogen receptor antagonism: structure-based identification of novel antiandrogens effective against multiple clinically relevant androgen receptor mutants. Chem. Biol. Drug Des. 79:300-12, 2012.
55. Liu W, Zhou J, Geng G, et al.: Antiandrogenic, maspin induction, and antiprostate cancer activities of tanshinone IIA and its novel derivatives with modification in ring A. J. Med. Chem. 55:971-5, 2012.
56. W. X., Zhang, J. W., Chen, Zhongguo Yaowu Huaxue Zazhi, 2000, 10(3), 161-163.
57. R. T., Clemens, M. P., Jennings, Chem. Commun., 2006, 2720-2721.
58. E. R., van Rijssel, P., van Delft, G., Lodder, H. S., Overkleeft, G. A., van der Marel, D. V., Filippov, J. D. C. Codée, Angew. Chem. Int. Ed. 2014, 53, 10381-10385.
59. D. Rennison; D., Conole; M. D. Tingle; J. P., Yang; C. T., Eason; M. A. Brimble, Bioorg. Med. Chem. Lett., 2013, 23, 6629-6635.
60. Ryan, James, et al.: Transaminase triggered aza-Michael approach for the enantioselective synthesis of piperidine scaffolds. Journal of the American Chemical Society, 138(49), 2016, 15798-15800.
61. Boros, Eric E., et al.: Synthesis and Antiviral Activity of 7-Benzyl-4-hydroxy-1, 5-naphthyridin-2 (1H)-one HIV Integrase Inhibitors. Journal of medicinal chemistry, 52(9), 2009, 2754-2761.
62. Khurana, J. M., & Sharma, P.: Chemoselective Reduction of α, β-Unsaturated Aldehydes, Ketones, Carboxylic Acids, and Esters with Nickel Boride in Methanol-Water. Bulletin of the Chemical Society of Japan, 77(3), 2004, 549-552.
63. Network TCGAR: Integrated genomic analyses of ovarian carcinoma. Nature. Jun. 29 2011; 474(7353):609-615.
64. Cekan P, Hasegawa K, Pan Y, et al.: RCC1-dependent activation of Ran accelerates cell cycle and DNA repair, inhibiting DNA damage-induced cell senescence. Mol Biol Cell. Apr. 15 2016; 27(8):1346-1357.

1. A compound of general formula IA below, or a pharmaceutically acceptable salt thereof, or a solvate or hydrate thereof 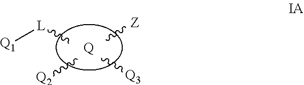
wherein:
Q is a 5 to 20-member single or multicyclo ring comprising at least one of O and S atoms;
L is a group comprising one or more of (CH₂), (CH), O, S, and C═X wherein X is O or S;
Z is CN; or (C═X)NR₁R₂ wherein X is O or S and R₁ and R₂ are each independently selected from H, alkyl, cycloalkyl, alkene, alkyne, aryl, alkylaryl, or together R₁ and R₂ form a 3 to 6-member ring which is optionally substituted with a substituent selected from alkyl, OH, SH, NH₂, a halogen atom, CN, NO₂ and SO₂; or a 3 to 6-member ring comprising one or more heteroatoms which are the same or different, optionally the ring is substituted with a substituent selected from COOR wherein R is a C₁-C₆-alkyl or cycloalkyl, alkoxy, alkyl, OH, SH, NH₂, a halogen atom, CN, NO₂ and SO₂; and
Q₁, Q₂ and Q₃ are each independently selected from alkyl, cycloalkyl, alkene, alkyne, aryl and alkylaryl, a 5 to 12-member single or bicyclo ring; optionally, the ring is substituted with a substituent selected from alkyl, cycloalkyl alkoxy, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂ and Se(═O)₂; also optionally, the ring comprises one or more heteroatoms which are the same or different; and
the heteroatom is selected from O, N, S and Se.

2. A compound according to claim 1 having the general formula IIA below 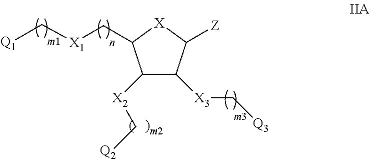
wherein:
Z is (C═X)NR₁R₂ or a 5-member ring comprising two heteroatoms which are different and the ring is substituted with COOR;
X is O, N or S;
n, m1, m2, and m3 are each independently an integer from 1 to 6; and
X₁, X₂ and X₃ are each independently O, N or S.

3. A compound according to claim 2 having the general formula IIIA below 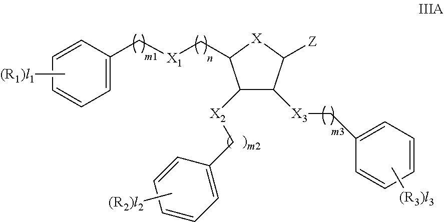
wherein:
Z is (C═X)NR₁R₂ or a 5-member ring comprising two heteroatoms which are different and the ring is substituted with COOR;
R₁, R₂ and R₃ are each independently H, alkyl, cycloalkyl, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂ and Se(═O)₂; and
I1, I2 and I3 are each independently an integer from 0 to 5.

4. A compound according to claim 2 or 3, wherein X is O.

5. A compound according to any one of claims 2 to 4, wherein Z is (C═S)NH₂ or

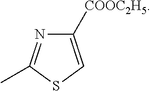

6. A compound according to any one of claims 2 to 5, wherein X₁, X₂ and X₃ are each O.

7. A compound according to any one of claims 2 to 6, wherein n, m1, m2 and m3 are each 1.

8. A compound according to any one of claims 3 to 7, wherein R₁, R₂ and R₃ are each independently a halogen atom; and I1, I2 and I3 are each 1.

9. A compound according to claim 1, wherein Q is the tetrahydrofuran ring.

10. A compound according to claim 1, which is selected from the group of compounds depicted in the Table 1 below
TABLE 1
Structures of M26 and M26 analogues of Class A

| ID | Structure |
|---|---|
| M26 | 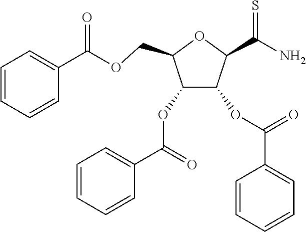 [SMILES: NC(=S)[C@@H]1O[C@H](COC(=O)c2ccccc2)[C@@H](OC(=O)c2ccccc2)[C@H]1OC(=O)c1ccccc1] |
| M33 | 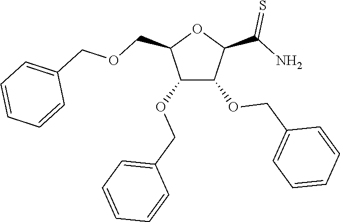 [SMILES: NC(=S)[C@@H]1O[C@H](COCc2ccccc2)[C@@H](OCc2ccccc2)[C@H]1OCc1ccccc1] |
| M34 | 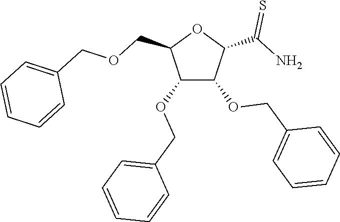 [SMILES: NC(=S)[C@H]1O[C@H](COCc2ccccc2)[C@@H](OCc2ccccc2)[C@H]1OCc1ccccc1] |
| M36 | 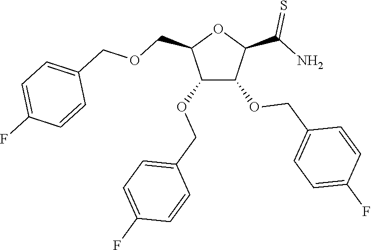 [SMILES: NC(=S)[C@@H]1O[C@H](COCc2ccc(F)cc2)[C@@H](OCc2ccc(F)cc2)[C@H]1OCc1ccc(F)cc1] |
| M39 | 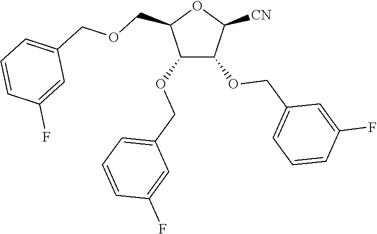 [SMILES: N#C[C@@H]1O[C@H](COCc2cccc(F)c2)[C@@H](OCc2cccc(F)c2)[C@H]1OCc1cccc(F)c1] |
| M40 | 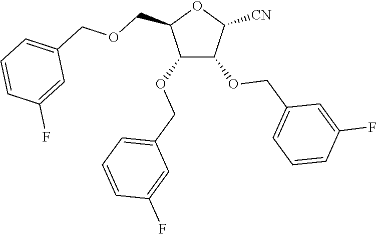 [SMILES: N#C[C@H]1O[C@H](COCc2cccc(F)c2)[C@@H](OCc2cccc(F)c2)[C@H]1OCc1cccc(F)c1] |
| M41 | 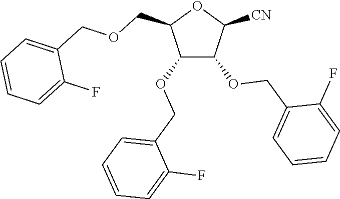 [SMILES: N#C[C@@H]1O[C@H](COCc2ccccc2F)[C@@H](OCc2ccccc2F)[C@H]1OCc1ccccc1F] |
| M42 | 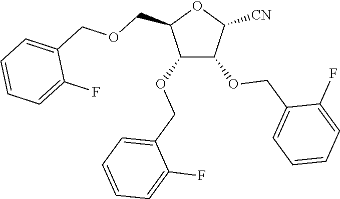 [SMILES: N#C[C@H]1O[C@H](COCc2ccccc2F)[C@@H](OCc2ccccc2F)[C@H]1OCc1ccccc1F] |
| M43 | 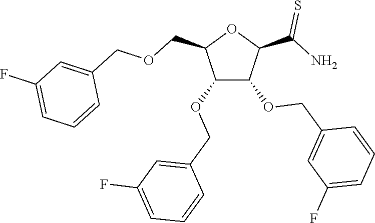 [SMILES: NC(=S)[C@@H]1O[C@H](COCc2cccc(F)c2)[C@@H](OCc2cccc(F)c2)[C@H]1OCc1cccc(F)c1] |
| M44 | 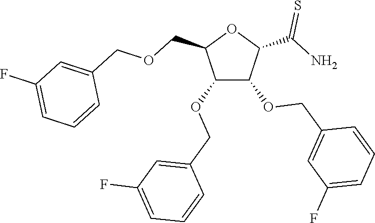 [SMILES: NC(=S)[C@H]1O[C@H](COCc2cccc(F)c2)[C@@H](OCc2cccc(F)c2)[C@H]1OCc1cccc(F)c1] |
| M45 | 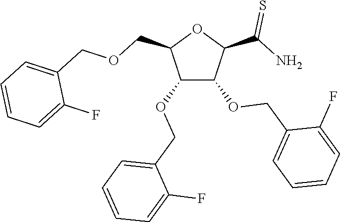 [SMILES: NC(=S)[C@@H]1O[C@H](COCc2ccccc2F)[C@@H](OCc2ccccc2F)[C@H]1OCc1ccccc1F] |
| M46 | 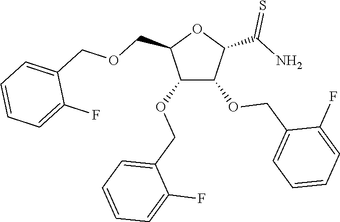 [SMILES: NC(=S)[C@H]1O[C@H](COCc2ccccc2F)[C@@H](OCc2ccccc2F)[C@H]1OCc1ccccc1F] |
| M57 | 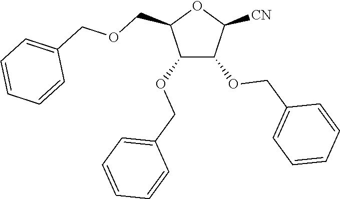 [SMILES: N#C[C@@H]1O[C@H](COCc2ccccc2)[C@@H](OCc2ccccc2)[C@H]1OCc1ccccc1] |
| M58 | 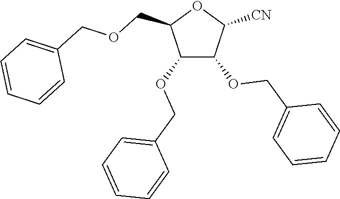 [SMILES: N#C[C@H]1O[C@H](COCc2ccccc2)[C@@H](OCc2ccccc2)[C@H]1OCc1ccccc1] |
| M59 | 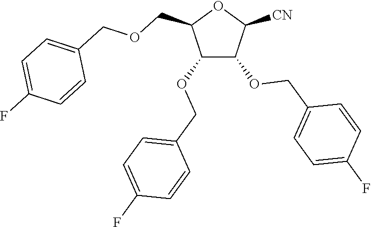 [SMILES: N#C[C@@H]1O[C@H](COCc2ccc(F)cc2)[C@@H](OCc2ccc(F)cc2)[C@H]1OCc1ccc(F)cc1] |
| M88 | 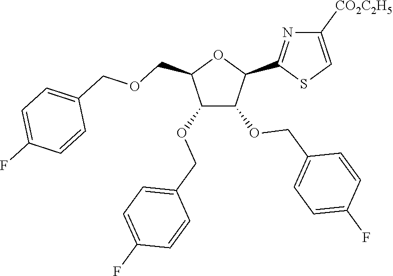 [SMILES: CCOC(=O)c1csc([C@@H]2O[C@H](COCc3ccc(F)cc3)[C@@H](OCc3ccc(F)cc3)[C@H]2OCc2ccc(F)cc2)n1] |

11. A compound according to claim 1, which is compound M36 depicted below

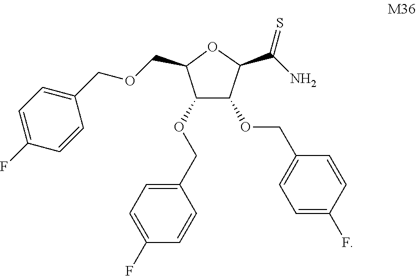 [SMILES: NC(=S)[C@@H]1O[C@H](COCc2ccc(F)cc2)[C@@H](OCc2ccc(F)cc2)[C@H]1OCc1ccc(F)cc1]

12. A compound according to claim 1, which is compound M88 depicted below

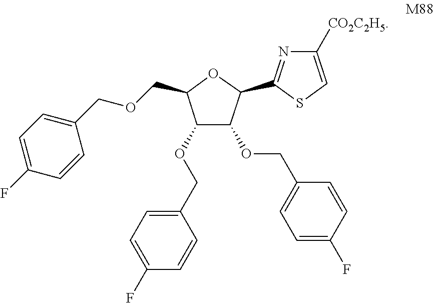 [SMILES: CCOC(=O)c1csc([C@@H]2O[C@H](COCc3ccc(F)cc3)[C@@H](OCc3ccc(F)cc3)[C@H]2OCc2ccc(F)cc2)n1]

13. A compound of general formula IB below, or a pharmaceutically acceptable salt thereof, or a solvate or hydrate thereof 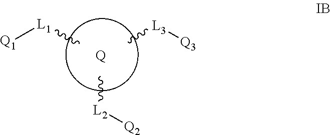
wherein:
Q is a 6 to 20-member single or multicyclo ring;
L₁, L₂ and L₃ are each independently a group comprising one or more of (CH₂), (CH), O, N, S and C═X wherein X is O or S, and NR₁R₂ wherein R₁ and R₂ are each independently selected from H, alkyl, cycloalkyl, alkene, alkyne, aryl, alkylaryl, or together R₁ and R₂ form a 3 to 6-member ring; and
Q₁, Q₂ and Q₃ are each independently selected from alkyl, cycloalkyl, alkene, alkyne, aryl and alkylaryl, a 5 to 12-member single or bicyclo ring; optionally, the ring is substituted with a substituent selected from alkyl, cycloalkyl alkoxy, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂ and Se(═O)₂; also optionally, the ring comprises one or more heteroatoms which are the same or different and selected from O, N, S and Se.

14. A compound according to claim 13 having the general formula IIB or IIB′ below 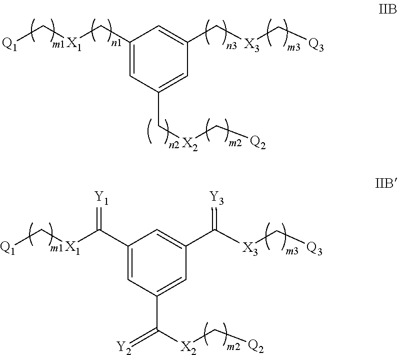
wherein:
n1, n2, n3, m1, m2, and m3 are each independently an integer from 0 to 6;
X₁, X₂ and X₃ are each independently selected from O; N; S; C═X wherein X is O or S; NR₁R₂ wherein R₁ and R₂ are each independently selected from selected from H, alkyl, cycloalkyl, alkene, alkyne, aryl, alkylaryl, or together R₁ and R₂ form a 3 to 6-member ring; (C═X)NR₁R₂ wherein X is O or S and R₁ and R₂ are each independently selected from selected from H, alkyl, cycloalkyl, alkene, alkyne, aryl, alkylaryl, or together R₁ and R₂ form a 3 to 6-member ring; and
Y₁, Y₂ and Y₃ are each independently selected from O, N and S.

15. A compound according to claim 13 or 14 having the general formula IIIB or IIIB′ below 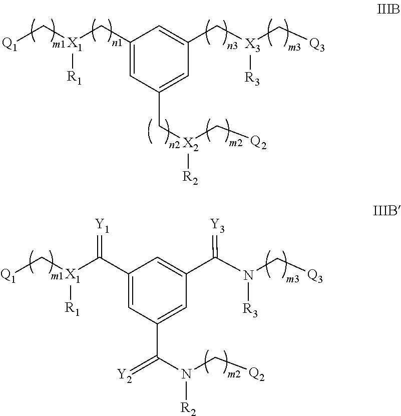
wherein:
X₁, X₂ and X₃ are each independently selected from O and N;
Y₁, Y₂ and Y₃ are each independently selected from O and S; and
R₁, R₂ and R₃ are each independently selected from H, alkyl, cycloalkyl, alkene, alkyne, aryl and alkylaryl.

16. A compound according to claim 14 or 15, wherein n1, n2, n3, m1, m2, and m3 are each 1.

17. A compound according to any one of claims 14 to 16, wherein X₁, X₂ and X₃ in IIB or IIIB are each N, and Y₁, Y₂ and Y₃ in IIB′ or IIIB′ are each O.

18. A compound according to any one of claims 14 to 17, wherein Q₁, Q₂ and Q₃ are each independently is a 5 or 6-member ring, optionally the ring comprises one or more heteroatoms selected from O, N, S and Se, and/or optionally the ring is substituted with one or more groups selected from C₁ to C₆ alkoxy and halogens.

19. A compound according to claim 13, wherein Q is the benzene ring.

20. A compound according to claim 13, which is selected from the group of compounds depicted in the Table 2 below
TABLE 2
Structures of M26 analogues of Class B~D

| ID | Structure |
|---|---|
| M47 | 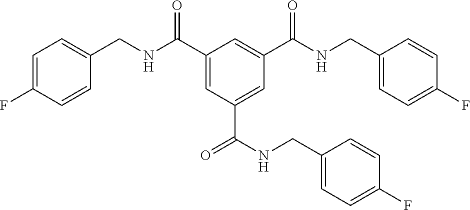 [SMILES: O=C(NCc1ccc(F)cc1)c1cc(C(=O)NCc2ccc(F)cc2)cc(C(=O)NCc2ccc(F)cc2)c1] |
| M48 | 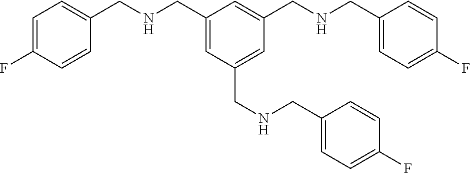 [SMILES: Fc1ccc(CNCc2cc(CNCc3ccc(F)cc3)cc(CNCc3ccc(F)cc3)c2)cc1] |
| M49 | 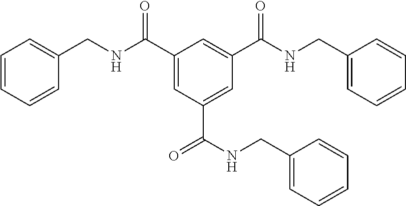 [SMILES: O=C(NCc1ccccc1)c1cc(C(=O)NCc2ccccc2)cc(C(=O)NCc2ccccc2)c1] |
| M50 | 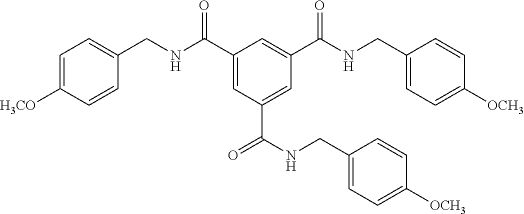 [SMILES: COc1ccc(CNC(=O)c2cc(C(=O)NCc3ccc(OC)cc3)cc(C(=O)NCc3ccc(OC)cc3)c2)cc1] |
| M51 | 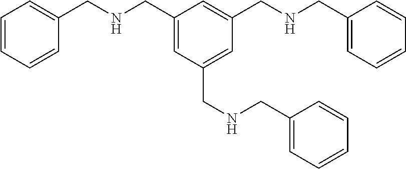 [SMILES: c1ccc(CNCc2cc(CNCc3ccccc3)cc(CNCc3ccccc3)c2)cc1] |
| M51S | 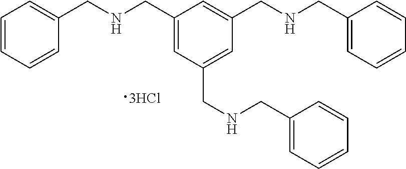 [SMILES: c1ccc(CNCc2cc(CNCc3ccccc3)cc(CNCc3ccccc3)c2)cc1] |
| M52 | 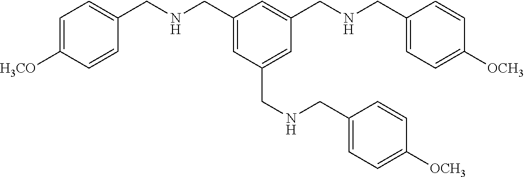 [SMILES: COc1ccc(CNCc2cc(CNCc3ccc(OC)cc3)cc(CNCc3ccc(OC)cc3)c2)cc1] |
| M53 | 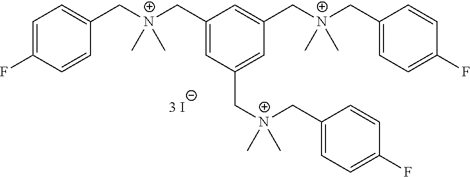 |
| M54 | 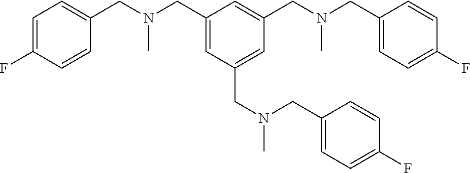 [SMILES: CN(Cc1ccc(F)cc1)Cc1cc(CN(C)Cc2ccc(F)cc2)cc(CN(C)Cc2ccc(F)cc2)c1] |
| M55 | 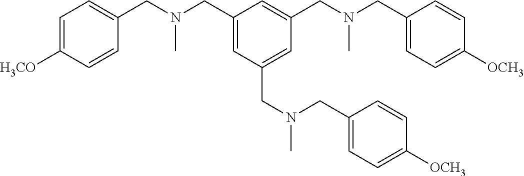 [SMILES: COc1ccc(CN(C)Cc2cc(CN(C)Cc3ccc(OC)cc3)cc(CN(C)Cc3ccc(OC)cc3)c2)cc1] |
| M56 | 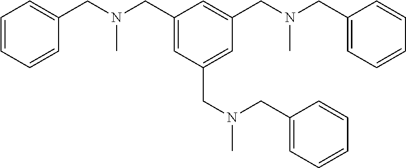 [SMILES: CN(Cc1ccccc1)Cc1cc(CN(C)Cc2ccccc2)cc(CN(C)Cc2ccccc2)c1] |
| M60 | 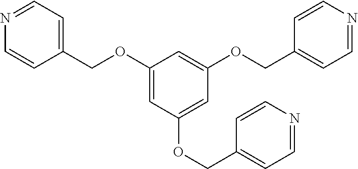 [SMILES: c1cc(COc2cc(OCc3ccncc3)cc(OCc3ccncc3)c2)ccn1] |
| M61 | 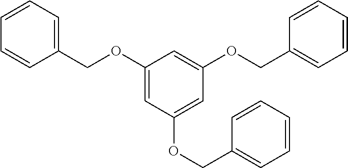 [SMILES: c1ccc(COc2cc(OCc3ccccc3)cc(OCc3ccccc3)c2)cc1] |
| M62 | 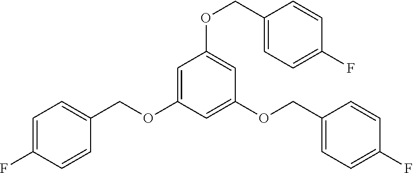 [SMILES: Fc1ccc(COc2cc(OCc3ccc(F)cc3)cc(OCc3ccc(F)cc3)c2)cc1] |
| M63 | 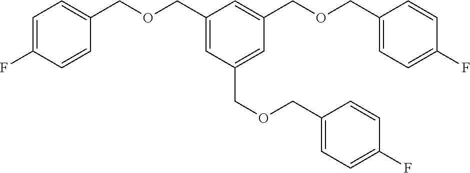 [SMILES: Fc1ccc(COCc2cc(COCc3ccc(F)cc3)cc(COCc3ccc(F)cc3)c2)cc1] |
| M64 | 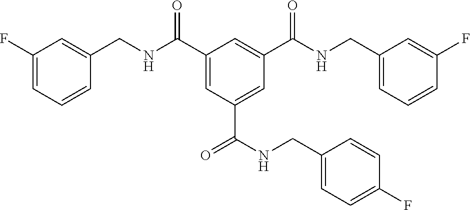 [SMILES: O=C(NCc1ccc(F)cc1)c1cc(C(=O)NCc2cccc(F)c2)cc(C(=O)NCc2cccc(F)c2)c1] |
| M65 | 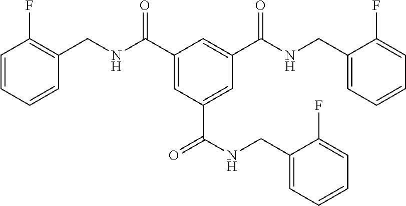 [SMILES: O=C(NCc1ccccc1F)c1cc(C(=O)NCc2ccccc2F)cc(C(=O)NCc2ccccc2F)c1] |
| M66 | 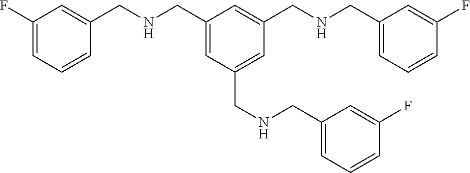 [SMILES: Fc1cccc(CNCc2cc(CNCc3cccc(F)c3)cc(CNCc3cccc(F)c3)c2)c1] |
| M67 | 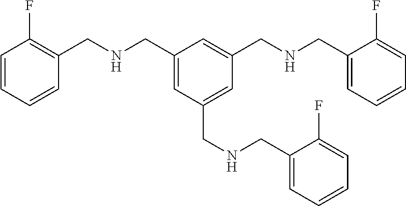 [SMILES: Fc1ccccc1CNCc1cc(CNCc2ccccc2F)cc(CNCc2ccccc2F)c1] |
| M68 | 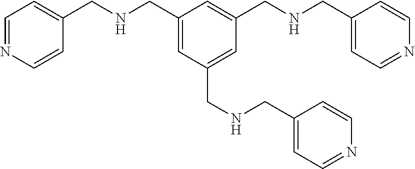 [SMILES: c1cc(CNCc2cc(CNCc3ccncc3)cc(CNCc3ccncc3)c2)ccn1] |
| M69 | 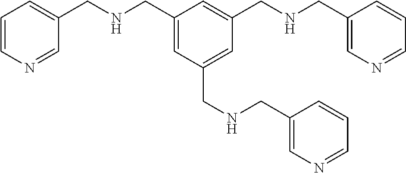 [SMILES: c1cncc(CNCc2cc(CNCc3cccnc3)cc(CNCc3cccnc3)c2)c1] |
| M69S | 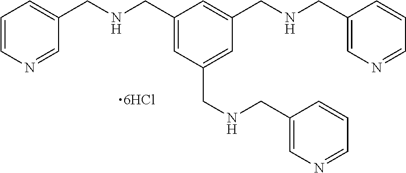 [SMILES: c1cncc(CNCc2cc(CNCc3cccnc3)cc(CNCc3cccnc3)c2)c1] |
| M70 | 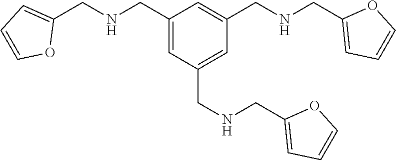 [SMILES: c1coc(CNCc2cc(CNCc3ccco3)cc(CNCc3ccco3)c2)c1] |
| M70S | 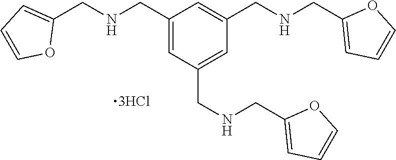 [SMILES: c1coc(CNCc2cc(CNCc3ccco3)cc(CNCc3ccco3)c2)c1] |
| M71 | 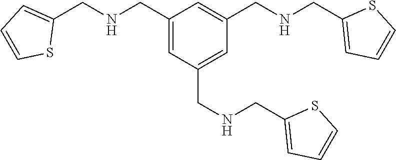 [SMILES: c1csc(CNCc2cc(CNCc3cccs3)cc(CNCc3cccs3)c2)c1] |
| M71S | 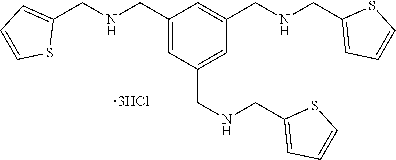 [SMILES: c1csc(CNCc2cc(CNCc3cccs3)cc(CNCc3cccs3)c2)c1] |
| M72S | 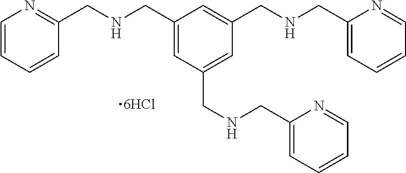 [SMILES: c1ccc(CNCc2cc(CNCc3ccccn3)cc(CNCc3ccccn3)c2)nc1] |
| M73 | 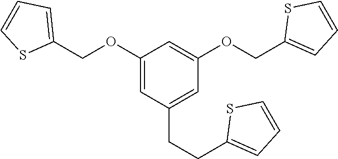 [SMILES: c1csc(CCc2cc(OCc3cccs3)cc(OCc3cccs3)c2)c1] |
| M75 | 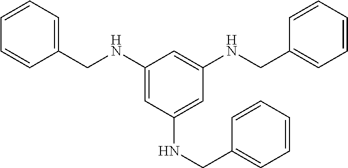 [SMILES: c1ccc(CNc2cc(NCc3ccccc3)cc(NCc3ccccc3)c2)cc1] |
| M75S | 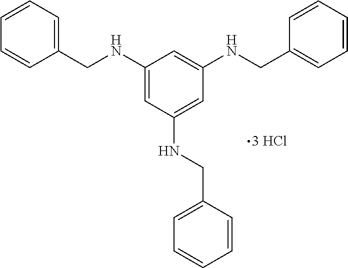 [SMILES: c1ccc(CNc2cc(NCc3ccccc3)cc(NCc3ccccc3)c2)cc1] |
| M76 | 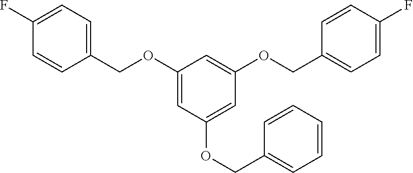 [SMILES: Fc1ccc(COc2cc(OCc3ccccc3)cc(OCc3ccc(F)cc3)c2)cc1] |
| M77 | 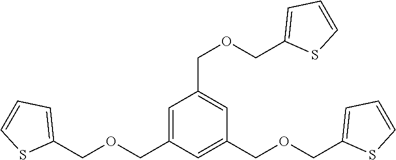 [SMILES: c1csc(COCc2cc(COCc3cccs3)cc(COCc3cccs3)c2)c1] |
| M78 | 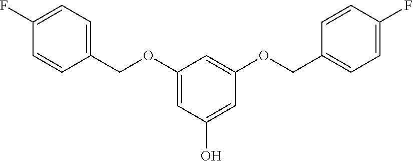 [SMILES: Oc1cc(OCc2ccc(F)cc2)cc(OCc2ccc(F)cc2)c1] |
| M79 | 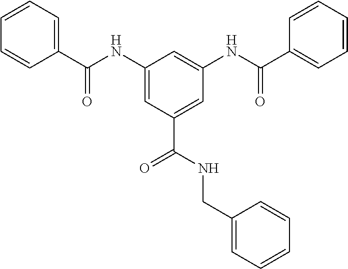 [SMILES: O=C(NCc1ccccc1)c1cc(NC(=O)c2ccccc2)cc(NC(=O)c2ccccc2)c1] |
| M80 | 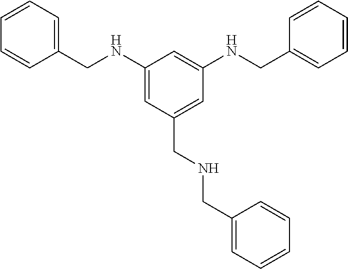 [SMILES: c1ccc(CNCc2cc(NCc3ccccc3)cc(NCc3ccccc3)c2)cc1] |
| M80S | 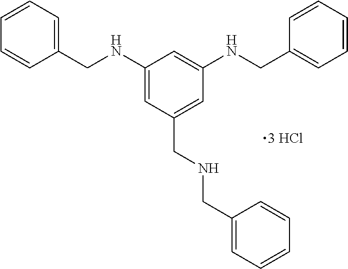 [SMILES: c1ccc(CNCc2cc(NCc3ccccc3)cc(NCc3ccccc3)c2)cc1] |
| M81 | 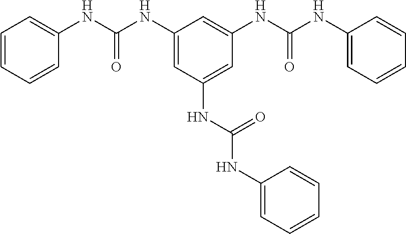 [SMILES: O=C(Nc1ccccc1)Nc1cc(NC(=O)Nc2ccccc2)cc(NC(=O)Nc2ccccc2)c1] |
| M82 | 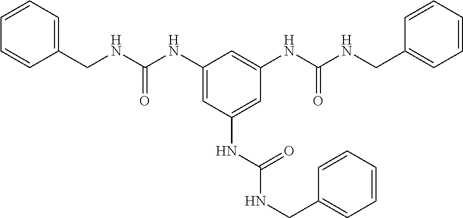 [SMILES: O=C(NCc1ccccc1)Nc1cc(NC(=O)NCc2ccccc2)cc(NC(=O)NCc2ccccc2)c1] |
| M83 | 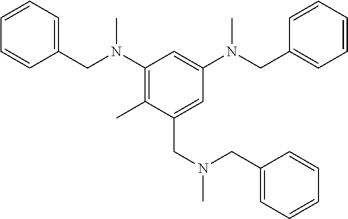 [SMILES: Cc1c(CN(C)Cc2ccccc2)cc(N(C)Cc2ccccc2)cc1N(C)Cc1ccccc1] |
| M84 | 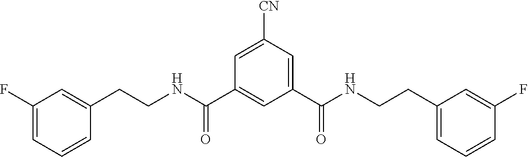 [SMILES: N#Cc1cc(C(=O)NCCc2cccc(F)c2)cc(C(=O)NCCc2cccc(F)c2)c1] |
| M85 | 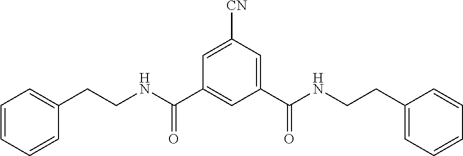 [SMILES: N#Cc1cc(C(=O)NCCc2ccccc2)cc(C(=O)NCCc2ccccc2)c1] |
| M86 | 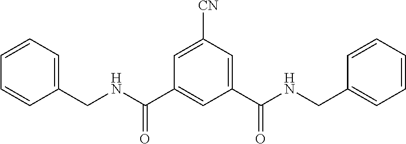 [SMILES: N#Cc1cc(C(=O)NCc2ccccc2)cc(C(=O)NCc2ccccc2)c1] |
| M87 | 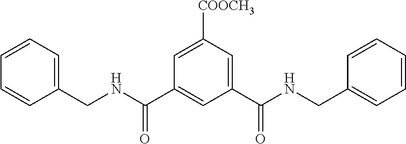 [SMILES: COC(=O)c1cc(C(=O)NCc2ccccc2)cc(C(=O)NCc2ccccc2)c1] |
| M89 | 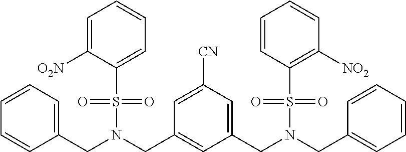 [SMILES: N#Cc1cc(CN(Cc2ccccc2)S(=O)(=O)c2ccccc2[N+](=O)[O-])cc(CN(Cc2ccccc2)S(=O)(=O)c2ccccc2[N+](=O)[O-])c1] |
| M90 | 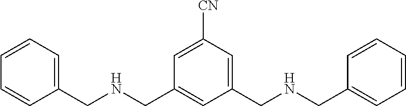 [SMILES: N#Cc1cc(CNCc2ccccc2)cc(CNCc2ccccc2)c1] |
| M91 | 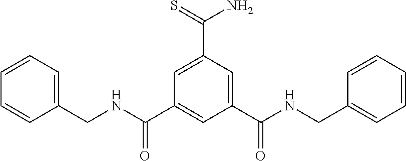 [SMILES: NC(=S)c1cc(C(=O)NCc2ccccc2)cc(C(=O)NCc2ccccc2)c1] |
| M92 | 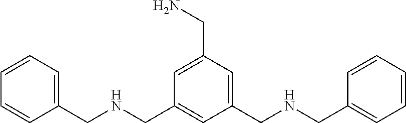 [SMILES: NCc1cc(CNCc2ccccc2)cc(CNCc2ccccc2)c1] |
| M93 | 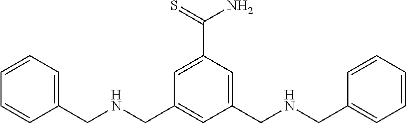 [SMILES: NC(=S)c1cc(CNCc2ccccc2)cc(CNCc2ccccc2)c1] |
| M94 | 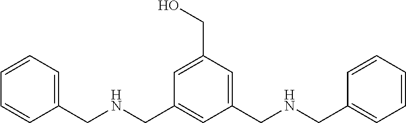 [SMILES: OCc1cc(CNCc2ccccc2)cc(CNCc2ccccc2)c1] |
| M95 | 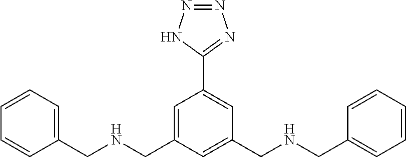 [SMILES: c1ccc(CNCc2cc(CNCc3ccccc3)cc(-c3nnn[nH]3)c2)cc1] |
| M96 | 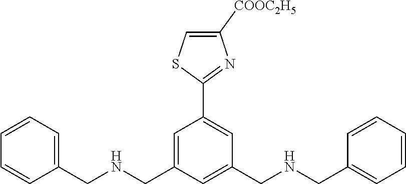 |
| M97 | 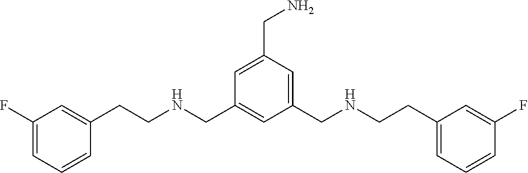 [SMILES: NCc1cc(CNCCc2cccc(F)c2)cc(CNCCc2cccc(F)c2)c1] |

21. A compound according to claim 13, which is compound M51 depicted below

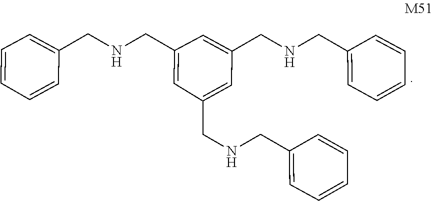 [SMILES: c1ccc(CNCc2cc(CNCc3ccccc3)cc(CNCc3ccccc3)c2)cc1]

22. A compound according to claim 13, which is compound M55 depicted below

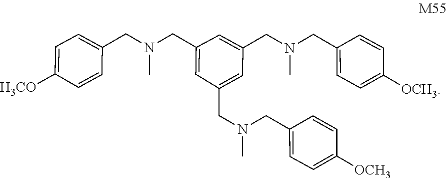 [SMILES: COc1ccc(CN(C)Cc2cc(CN(C)Cc3ccc(OC)cc3)cc(CN(C)Cc3ccc(OC)cc3)c2)cc1]

23. A compound according to claim 13, which is compound M66 depicted below

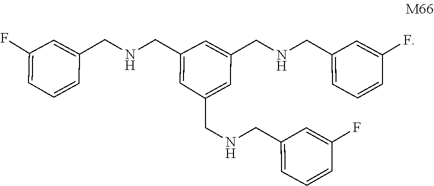 [SMILES: Fc1cccc(CNCc2cc(CNCc3cccc(F)c3)cc(CNCc3cccc(F)c3)c2)c1]

24. A compound of general formula IC below, or a pharmaceutically acceptable salt thereof, or a solvate or hydrate thereof 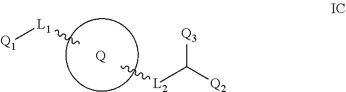
wherein:
Q is a 6 to 20-member single or multicyclo ring comprising at least two N atoms;
L₁ is a group comprising one or more of (CH₂), (CH), O, N, S and C═X wherein X is O or S, and L₁ is attached to one of the at least two N atoms;
L₂ is present or absent and is a group comprising one or more of (CH₂), (CH), O, N, S and C═X wherein X is O or S, and L₂ is attached to another one of the at least two N atoms;
Q₁, Q₂ and Q₃ are each independently selected from H, alkyl, cycloalkyl, alkene, alkyne, aryl and alkylaryl, a 5 to 12-member single or bicyclo ring; optionally, the ring is substituted with a substituent selected from alkyl, cycloalkyl alkoxy, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂, Se(═O)₂ and N(HNC═X)₂(Ph-halogen(s))₂ wherein X is O or S; also optionally, the ring comprises one or more heteroatoms which are the same or different and selected from O, N, S and Se.

25. A compound according to claim 24 having the general formula IIC below 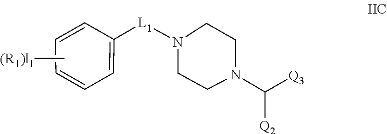
wherein:
Q₂ and Q₃ are each independently selected from alkyl, cycloalkyl, alkene, alkyne, aryl and alkylaryl, a 5 to 12-member single or bicyclo ring; optionally, the ring is substituted with a substituent selected from alkyl, cycloalkyl alkoxy, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂ and Se(═O)₂; also optionally, the ring comprises one or more heteroatoms which are the same or different and selected from O, N, S and Se;
R₁ is selected from H, alkyl, cycloalkyl, alkylaryl, alkoxy, thioalkoxy, aryl, aryloxy, thioaryloxy, alkylaryloxy, thioalkylaryloxy, OH, SH, NH₂, a halogen atom, a halogeno alkyl, a halogeno alkoxy, a halogeno thioalkoxy, CN, NO₂, S(═O)₂ and Se(═O)₂; and
I1 is an integer from 0 to 5.

26. A compound according to claim 25, having the general formula IIIC below 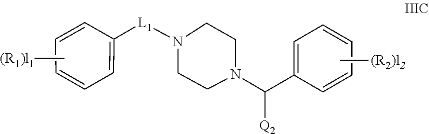
wherein:
R₂ is as defined for R₁; and
I2 is as defined for I1.

27. A compound according to any one of claims 24 to 26, wherein L₁ is (CH₂)ₙ wherein n is an integer from 0 to 12.

28. A compound according to claim 25 or 26, wherein Q₂ is a cycloalkyl or alkylaryl.

29. A compound according to claim 24, wherein Q is the piperazine ring.

30. A compound according to claim 24, which is selected from the group of compounds depicted in the Table 3 or Table 4 below
TABLE 3
Structures of R20 and R20 analogues of Class A

| ID | Structure |
|---|---|
| R20 | 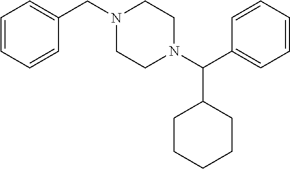 |
| QR20 | 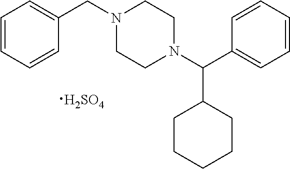 |
| R21 | 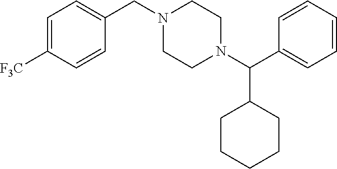 |
| R22 | 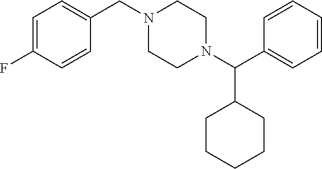 |
| R37 | 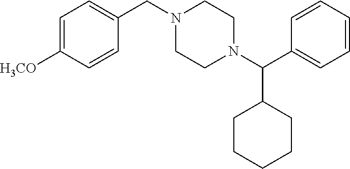 |
| R38 | 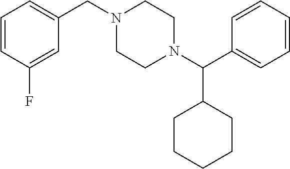 |
| R39 | 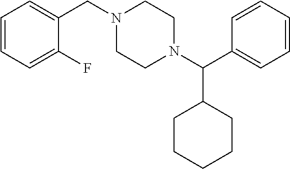 |
| R40 | 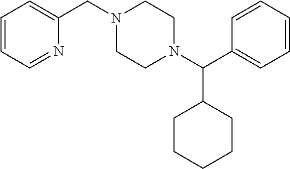 |
| R41 | 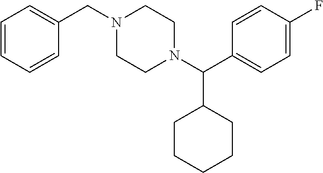 |
| R42 | 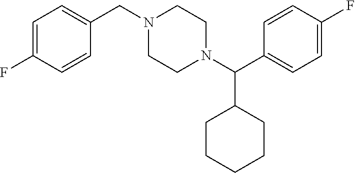 |
| R43 | 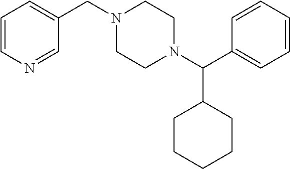 |
| R44 | 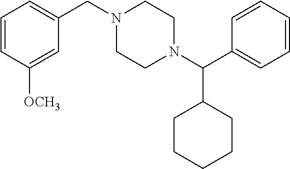 |
| R47 | 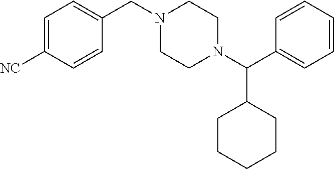 |
| R48 | 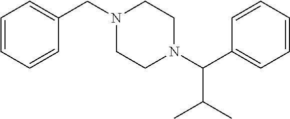 |
| R49 | 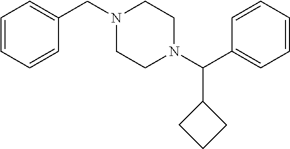 |
| R50 | 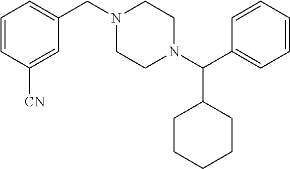 |
| R52 | 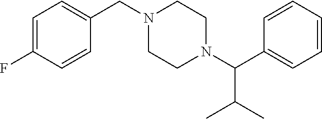 |
| R53 | 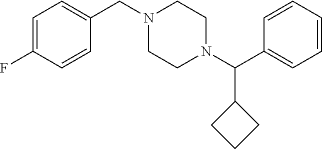 |
| R56 | 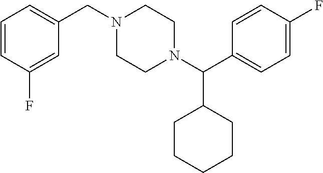 |
| R57 | 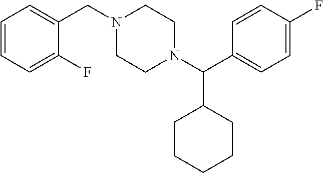 |
| R58 | 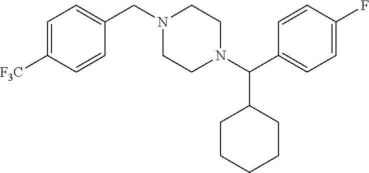 |
| R59 | 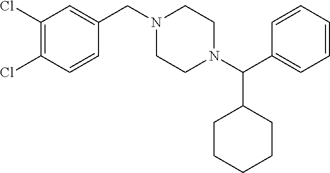 |
| R60 | 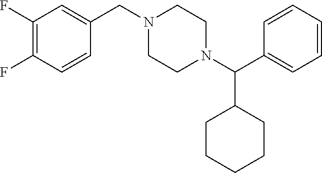 |
| R61 | 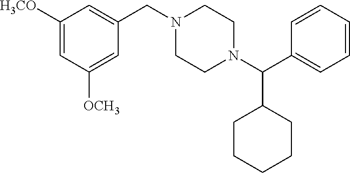 |
| R62 | 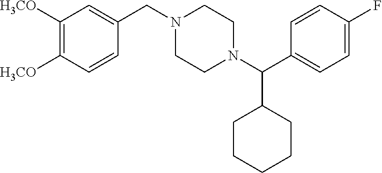 |
| R67 | 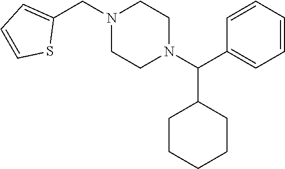 |
| R68 | 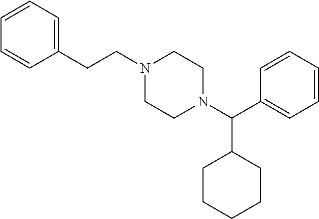 |
| R69 | 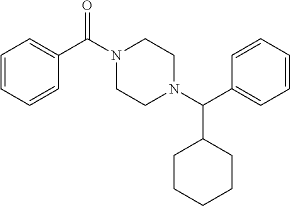 |
| R70 | 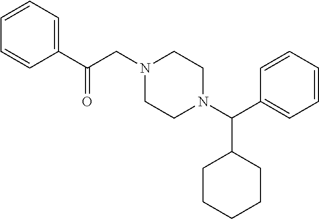 |
| R71 | 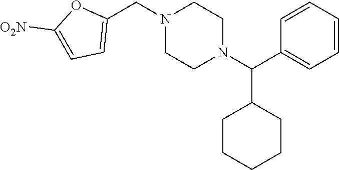 |

TABLE 4
Structures of R20 analogues of Class B

| | |
|---|---|
| R27 | 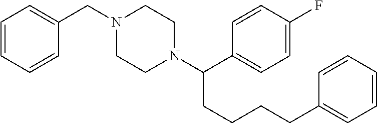 |
| R35 | 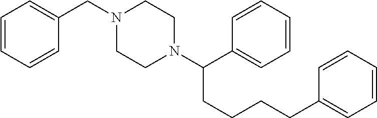 |
| R36 | 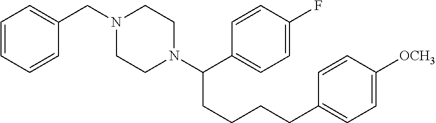 |
| R45 | 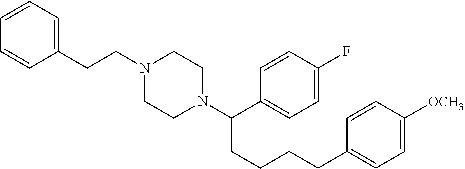 |
| R46 | 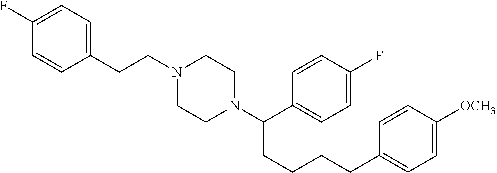 |
| R51 | 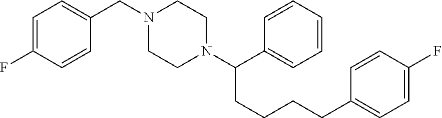 |
| R54 | 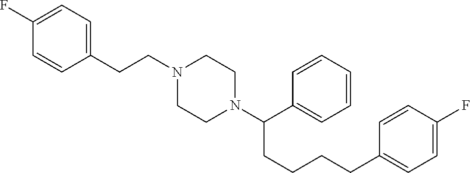 |
| R55 | 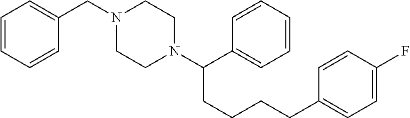 |

31. A compound according to claim 24, which is compound R20 depicted below

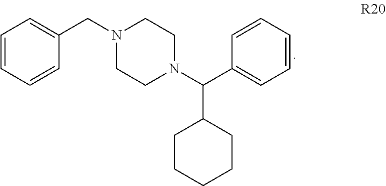 [SMILES: c1ccc(CN2CCN(C(c3ccccc3)C3CCCCC3)CC2)cc1]

32. A compound according to claim 24, which is compound QR20 depicted below

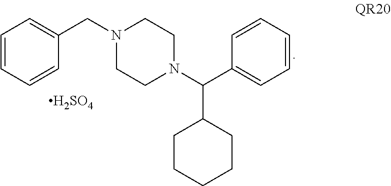

33. A compound according to claim 24 having the general formula IIC′ below 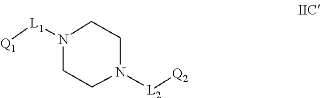
wherein:
Q₁ and Q₂ are each independently selected from alkyl, cycloalkyl, alkene, alkyne, aryl and alkylaryl, a 5 to 12-member single or bicyclo ring; optionally, the ring is substituted with a substituent selected from alkyl, cycloalkyl alkoxy, alkoxy, thioalkoxy, aryl, aryloxy, thioaryloxy, alkyaryloxy, thioalkylaryloxy, OH, SH, NH₂, a halogen atom, a halogeno alkyl, a halogeno alkoxy, a halogeno thioalkoxy, CN, NO₂, S(═O)₂, Se(═O)₂ and N(HNC═X)₂(Ph-halogen(s))₂ wherein X is O or S; also optionally, the ring comprises one or more heteroatom which are the same or different and wherein the heteroatom is selected from O, N, S and Se.

34. A compound according to claim 33 having the general formula IIIC′ below 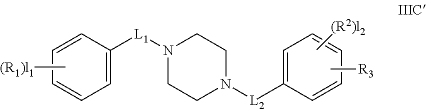
wherein:
R₁, R₂ and R₃ are each independently selected from H, alkyl, cycloalkyl, alkylaryl, alkoxy, thioalkoxy, aryl, aryloxy, thioaryloxy, alkylaryloxy, thioalkylaryloxy, OH, SH, NH₂, a halogen atom, a halogeno alkyl, a halogeno alkoxy, a halogeno thioalkoxy, CN, NO₂, S(═O)₂, Se(═O)₂ and N(HNC═X)₂(Ph-halogen(s))₂ wherein X is O or S; and
I1 is an integer from 0 to 5; and
I2 is an integer from 0 to 4.

35. A compound according to claim 34 having the general formula IVC′ below 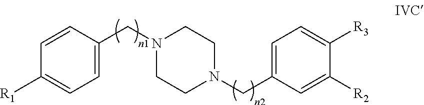
wherein:
n1 and n2 are each independently an integer from 0 to 12.

36. A compound according to claim 34, wherein L₁ and L₂ are each independently (CH₂)ₙ wherein n is an integer from 0 to 12.

37. A compound according to claim 36, wherein n1 and n2 are each independently an integer from 1 to 3.

38. A compound according to claim 36, wherein R₁ and R₃ are each independently an alkoxy or a halogen.

39. A compound according to claim 36, wherein R₂ is N(HNC═X)₂(Ph-halogen(s))₂ wherein X is O or S.

40. A compound according to claim 24, wherein Q is the piperazine ring.

41. A compound according to claim 24, which is selected from the group of compounds depicted in the Table 5 below
TABLE 5
Structures of R20 analogues of Class C

| ID | Structure |
|---|---|
| R28 | 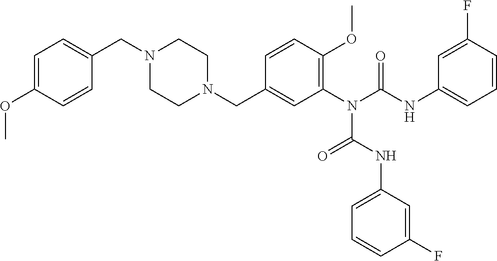 |
| R29 | 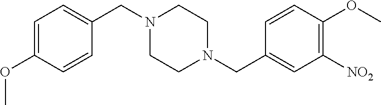 |
| R30 | 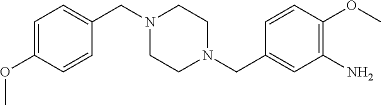 |
| R31 | 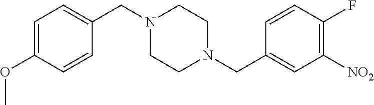 |
| R32 | 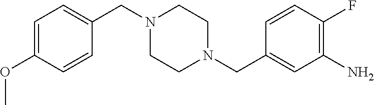 |
| R33 | 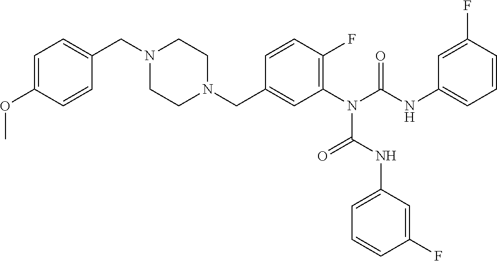 |
| R64 | 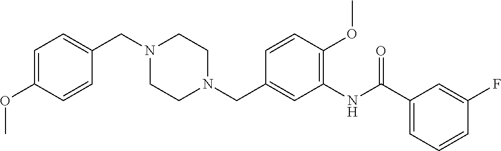 |
| R65 | 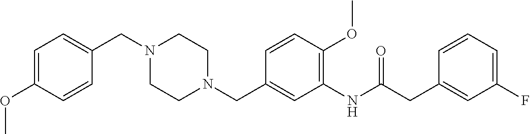 |
| R66 | 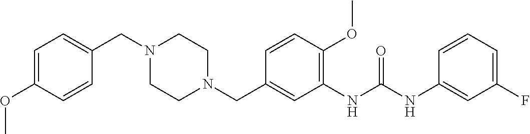 |
| 1156 | 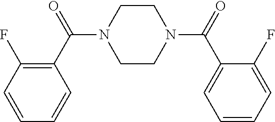 [SMILES: O=C(c1ccccc1F)N1CCN(C(=O)c2ccccc2F)CC1] |
| 1157 | 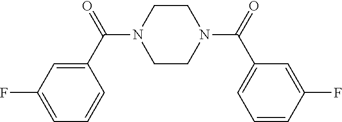 [SMILES: O=C(c1cccc(F)c1)N1CCN(C(=O)c2cccc(F)c2)CC1] |
| 1158 | 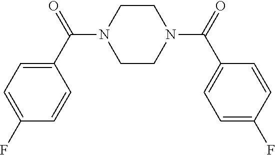 [SMILES: O=C(c1ccc(F)cc1)N1CCN(C(=O)c2ccc(F)cc2)CC1] |
| 1279 | 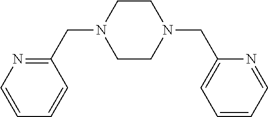 [SMILES: c1ccc(CN2CCN(Cc3ccccn3)CC2)nc1] |
| 1292 | 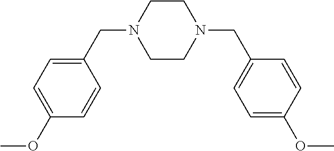 |
| 1293 | 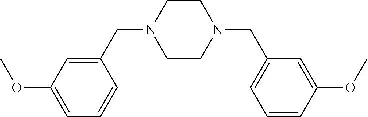 |
| 1294 | 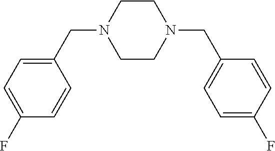 [SMILES: Fc1ccc(CN2CCN(Cc3ccc(F)cc3)CC2)cc1] |
| 1295 | 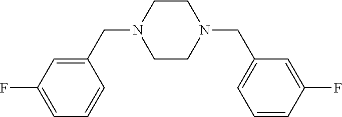 [SMILES: Fc1cccc(CN2CCN(Cc3cccc(F)c3)CC2)c1] |
| 1336 | 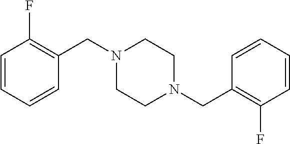 [SMILES: Fc1ccccc1CN1CCN(Cc2ccccc2F)CC1] |
| 1337 | 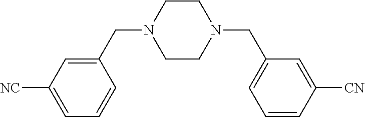 [SMILES: N#Cc1cccc(CN2CCN(Cc3cccc(C#N)c3)CC2)c1] |
| 1338 | 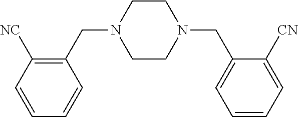 [SMILES: N#Cc1ccccc1CN1CCN(Cc2ccccc2C#N)CC1] |
| 1339 | 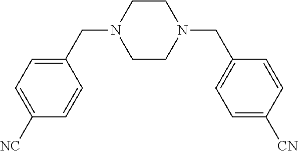 [SMILES: N#Cc1ccc(CN2CCN(Cc3ccc(C#N)cc3)CC2)cc1] |
| 1365 | 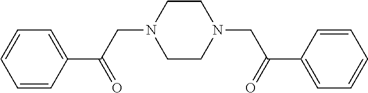 [SMILES: O=C(CN1CCN(CC(=O)c2ccccc2)CC1)c1ccccc1] |

42. A compound according to claim 35, which is compound R28 depicted below

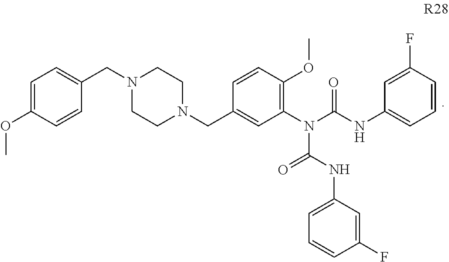 [SMILES: COc1ccc(CN2CCN(Cc3ccc(OC)c(N(C(=O)Nc4cccc(F)c4)C(=O)Nc4cccc(F)c4)c3)CC2)cc1]

43. A pharmaceutical composition comprising a compound as defined in any one of claims 1 to 42, and a pharmaceutically acceptable carrier.

44. A kit comprising a compound as defined in any one of claims 1 to 42 and/or a pharmaceutical composition as defined in claim 43, another therapeutic agent, and instructions for use in the treatment of a medical condition involving Ran GTPase.

45. A kit according to claim 44, wherein the other therapeutic agent comprises a DNA damaging agent such as carboplatin and/or an inhibitor of poly ADP ribase polymerase (PARP) such as olaparib.

46. A compound according to any one of claims 1 to 42, which inhibits Ran GTPase.

47. A method of treating a medical condition involving Ran GTPase, comprising administering to a subject a therapeutically effective amount of a compound as defined in any one of claims 1 to 42 or a pharmaceutical composition as defined in claim 43.

48. A method of treating a medical condition involving Ran GTPase, comprising administering to a subject a therapeutically effective amount of compound M26, V188, 1292 or a pharmaceutical composition comprising same.

49. A method according to claim 47 or 48, wherein the medical condition is a medical condition with immune disorder.

50. A method according to any one of claims 47 to 49, wherein the medical condition is cancer including ovarian cancer, breast cancer, pancreatic cancer, colorectal cancer and a cancer embodying aneuploidy.

51. A method according to any one of claims 47 to 50, further comprising treating the subject with a second therapy.

52. A method according to claim 51, wherein the second therapy comprises a DNA damaging agent such as carboplatin and/or an inhibitor of poly ADP ribase polymerase (PARP) such as olaparib.

53. A method according to any one of claims 47 to 51, wherein the compound is administered orally, intravenously, intra-arterially, subcutaneously, topically or intramuscularly.

54. A method according to claim 50, wherein the cancer is primary or multi-drug resistant, metastatic and/or recurrent.

55. A method according to claim 50 or 54, wherein the method comprises inhibiting cancer growth, killing cancer cells, reducing tumor burden, reducing tumor size, improving the subject's quality of life and/or prolonging the subject's length of life.

56. A method according to any one of claims 43 to 50, wherein the subject is human.

57. A method according to any one of claims 47 to 56, wherein the subject is a non-human animal.

58. Use of a compound as defined in any one of claims 1 to 42 or a pharmaceutical composition as defined in claim 43, for treating in a subject, a medical condition involving Ran GTPase.

59. Use of compound M26, V188, 1292 or a pharmaceutical composition comprising same, for treating in a subject, a medical condition involving Ran GTPase.

60. Use of a compound as defined in any one of claims 1 to 42, in the manufacture of a medicament for treating a medical condition involving Ran GTPase.

61. Use of compound M26, V188 or 1292, in the manufacture of a medicament for treating a medical condition involving Ran GTPase.

62. A compound as defined in any one of claims 1 to 42, for use in the treatment of a medical condition that involves Ran GTPase.

63. A pharmaceutical composition as defined in claim 43, for use in the treatment of a medical condition that involves Ran GTPase.
